(19) United States
(12) Patent Application Publication
(10) Pub. No.: US 2024/0208987 A1
(43) Pub. Date: Jun. 27, 2024

(54) EXATECAN-DERIVED ADC LINKER-PAYLOADS, PHARMACEUTICAL COMPOSITIONS, AND USES THEREOF

(71) Applicant: Merck Sharp & Dohme LLC, Rahway, NJ (US)

(72) Inventors: Vlad Bacauanu, San Francisco, CA (US); Manoj B. Charati, Redwood City, CA (US); Sijie Chen, Millbrae, CA (US); Rebecca Elizabeth Johnson, Oakland, CA (US); Simon B. Lang, San Francisco, CA (US); Christian L. Morales, Belmont, CA (US); Ryan Quiroz, San Francisco, CA (US); W. Michael Seganish, Castro Valley, CA (US); Nancy Zepeda, San Francisco, CA (US)

(73) Assignee: Merck Sharp & Dohme LLC, Rahway, NJ (US)

(21) Appl. No.: 18/491,850

(22) Filed: Oct. 23, 2023

Related U.S. Application Data
(60) Provisional application No. 63/419,150, filed on Oct. 25, 2022.

(51) Int. Cl. C07D 491/22; A61K 47/65; A61K 47/68

(52) U.S. Cl. CPC C07D 491/22; A61K 47/65; A61K 47/68037

(57) ABSTRACT

The present disclosure is directed to linker-payloads, and salts (including pharmaceutically acceptable salts), solvates, or stereoisomers thereof, comprising a structure of formula I:

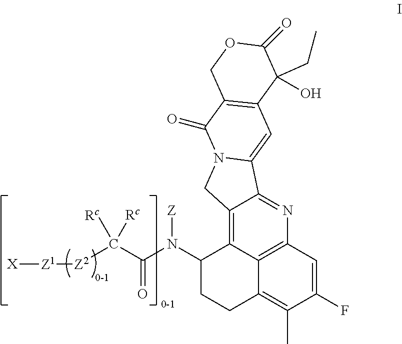

The disclosure is also directed to pharmaceutical compositions comprising these compounds and the use of these compounds, intermediates thereof, and compositions in the prevention or treatment of cancers and/or tumors.

### CROSS-REFERENCE TO RELATED APPLICATIONS

This application claims the benefit of priority to U.S. Provisional Application No. 63/419,150, filed Oct. 25, 2022, the disclosure of which is incorporated herein by its entirety.

### BACKGROUND OF THE DISCLOSURE

This disclosure represents a class of linker exatecan (camptothecin)-derived linker-payloads with utility for conjugation to antibodies or other targeting moieties to generate antibody-drug conjugates (ADCs), or other targeting ligand conjugates, for oncology indications. The compounds comprise topoisomerase-1 inhibitors derived from the exatecan scaffold connected to novel linker structures, which, when conjugated to a targeting moiety, are cytotoxic. Documents reporting the use of the camptothecin derivative, exatecan (chemical name: (1S,9S)-1-amino-9-ethyl-5-fluoro-2,3-dihydro-9-hydroxy-4-methyl-1H,12H-benzo[de]pyrano[3′,4:6,7]imidazo[1,2-b] quinoline-10,13(9H,15H)-dione) are disclosed in WO2014057687, U.S. Ser. No. 11/103,593, U.S. Pat. Nos. 9,808,537, 7,091,186, US 2010/0062008, WO2015057699, Clinical Cancer Research (2016) 22 (20): 5097-5108, and Cancer Sci (2016) 107: 1039-1046. See also WO2022068878, WO2017062271, CN113816969, CN112125915, and US20210353764.

### SUMMARY OF THE DISCLOSURE

Disclosed is a class of linker camptothecin-derived-payload compounds, wherein the linker structures contain a maleimide attached to a peptide linker, with variation on the amino acid sequence, optional incorporation of PEG units, and terminating with a hemiaminal or p-aminobenzyl carbamate (PABC) connection to a camptothecin-derived payload. Utility of these linker camptothecin-derived payload compounds is demonstrated by conjugation to cysteine residues in various targeting moieties, such as antibodies, to yield antibody drug conjugates (ADCs), which show favorable physical-chemical properties and high target-mediated potency. Thus, another embodiment of the disclosure is realized by ADCs disclosed herein. The linker-payloads of the present disclosure provide for potent and novel ADCs active across multiple cancer cell lines, and thereby demonstrate broad utility to be conjugated to several antibodies or other targeting moieties while still retaining favorable properties and efficacy. Exemplary ADCs from the linker-payloads described herein are also detailed.

The instant compounds are cytotoxic and can be applied as chemotherapeutic drugs in oncologic settings, for example as an anti-tumor agent. The compounds are potent, novel in structure, and active across multiple cancer cell lines.

### DETAILED DESCRIPTION OF THE DISCLOSURE

For each of the following embodiments, any variable not explicitly defined in the embodiment is as defined in Formula (I). In each of the embodiments described herein, each variable is selected independently of the other unless otherwise noted.

This disclosure is directed to a class of linker camptothecin-derived payload compounds, wherein the linker structures contain a maleimide attached to a peptide linker, with variation on the amino acid sequence and the optional incorporation of PEG units and terminating with a hemiaminal connection to a camptothecin-derived payload. An embodiment of the disclosure relates to a class of linker camptothecin-derived-payload compounds, wherein the linker structures contain a maleimide attached to a peptide linker, with variation on the amino acid sequence and the optional incorporation of PEG units and terminating with a PABC connection to a camptothecin-derived payload. Another embodiment of the disclosure relates to compounds where the linker camptothecin-derived payload compounds is conjugated to a targeting moiety that has a free cysteine group, including antibodies, proteins, peptides, polypeptides, and engineered antibodies. Still another embodiment of the disclosure relates to the maleimide-containing linker group.

In another embodiment, the present disclosure provides linker camptothecin-derived-payload compounds (also referred to as linker-payload(s)), and pharmaceutically acceptable salts, solvates, or stereoisomer thereof, comprising a structure of formula I:

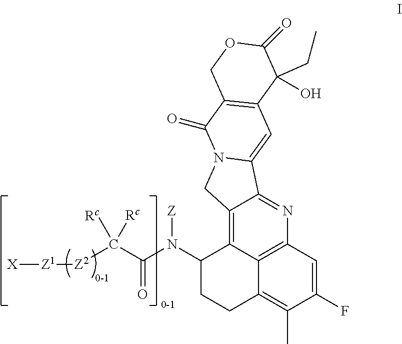

wherein:
Z is selected from hydrogen and —CH₂C(R^{x})(R^{y})CHF₂;
Z¹ is selected from —NH— and —O—;
Z² is absent or selected from —CR^{b}R^{b}—, —CH₂CR^{b}R^{b}—, and —CR^{b}R^{b}CH₂—;
each R^{b} is independently selected from hydrogen, —C₁₋₆ alkyl, and hydroxyl;
or two adjacent R^{b} combine to form spirocycloalkyl;
each R^{c} is independently selected from hydrogen, —C₁₋₆ alkyl, halogen, and hydroxyl; or two adjacent R^{c} combine to form spirocycloalkyl;
R^{x} and R^{y} are independently selected from hydrogen, C₁₋₆ alkyl, C₁₋₃ haloalkyl, halogen, hydroxyl, and —C₁₋₆ alkyl-OH;
or R^{x} and R^{y} combine to form a C₃₋₆cycloalkyl, or spirocycloalkyl;
X is a linking group selected from:

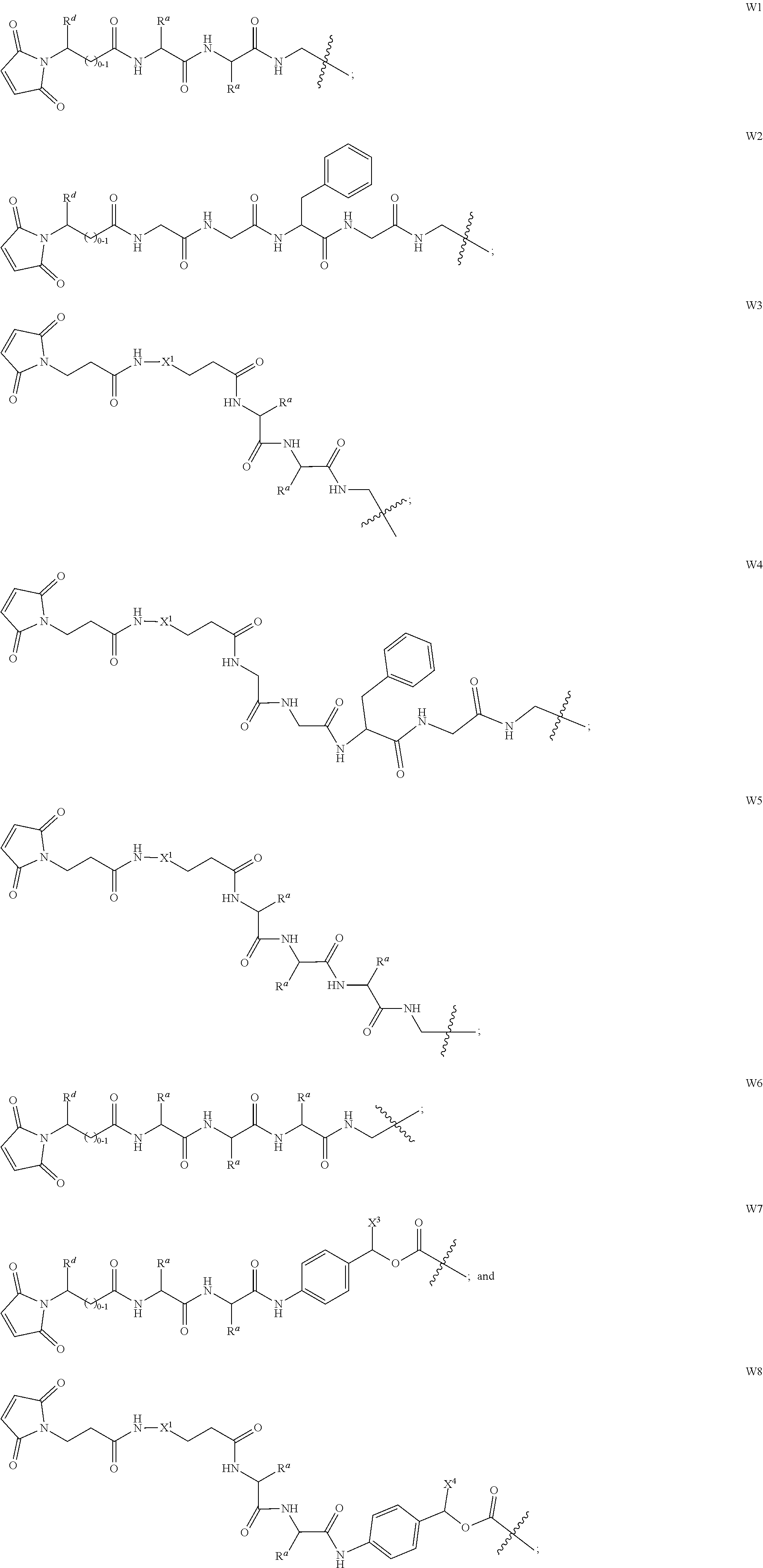

[symbol] represents the point of attachment to structural Formula I;
X¹ is a polyethylene glycol (PEG);
X³ is hydrogen or —C(O)NR^{a}R^{z},
X⁴ is hydrogen or

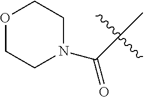 [SMILES: *C(=O)N1CCOCC1]

R^{a} are R^{z} are independently selected from hydrogen, C₁₋₆ alkyl, and X¹, or R^{a} and R^{z} combine to form a C₃₋₁₀ cycloalkyl, or a 3 to 10 membered heterocyclyl;
R^{d} is hydrogen, —CH₂NHC(O)X¹ or —CH₂NHC(O)X¹Q; and
Q is C₁₋₆ alkyl or hydrogen.

Another aspect of this embodiment is realized when X¹ is a PEG of 1 to 24 subunits. Another aspect of this embodiment is realized when X¹ is a PEG of 1 to 12 subunits. Another aspect of this embodiment is realized when X¹ is a PEG that terminates in an OH or OMe group.

An embodiment of this disclosure is realized when Z is hydrogen.

Another embodiment of this disclosure is realized when Z is —CH₂C(R^{x})(R^{y})CHF₂—. A subembodiment of this aspect of the disclosure is realized when R^{x} and R^{y} are independently selected from fluorine, chlorine, methyl, ethyl, and hydrogen. Another subembodiment of this aspect of the disclosure is realized when R^{x} and R^{y} are both fluorine. Another subembodiment of this aspect of the disclosure is realized when R^{x} and R^{y} are both methyl. Another subembodiment of this aspect of the disclosure is realized when one of R^{x} and R^{y} is methyl and the other fluorine.

Another embodiment of this disclosure is realized when each R^{c} is independently selected from hydrogen, OH, CH₃, CH₂OH, CHF₂, CH₂F, CF₃, fluorine, and chlorine. A subembodiment of this aspect of the disclosure is realized when one R^{c} is hydrogen and the other is OH. Another subembodiment of this aspect of the disclosure is realized when one R^{c} is hydrogen and the other is CH₃. Another subembodiment of this aspect of the disclosure is realized when one R^{c} is hydrogen and the other is CH₂OH. Another subembodiment of this aspect of the disclosure is realized when one R^{c} is CH₃ and the other is CHF₂. Another subembodiment of this aspect of the disclosure is realized when one R^{c} is hydrogen and the other is OH. A subembodiment of this aspect of the disclosure is realized when both R^{c} are CH₃. A subembodiment of this aspect of the disclosure is realized when both R^{c} are fluorine. A subembodiment of this aspect of the disclosure is realized when both R^{c} are hydrogen.

Another embodiment of this disclosure is realized when both R^{c} combine to C₃₋₆ spirocycloalkyl. A subembodiment of this aspect of the disclosure is realized when the spirocyclopropyl, spirocyclobutyl, spirocyclopentyl and spirocyclohexyl. Another subembodiment of this aspect of the disclosure is realized when the spirocycloalkyl is cyclopropyl.

Another embodiment of Formula I is realized when Q is C₁₋₆ alkyl and the alkyl is selected from methyl, ethyl, propyl, butyl or hexyl. A subembodiment of this aspect of the disclosure is realized when Q is C₁₋₆ alkyl and the alkyl is methyl.

Another embodiment of Formula V is realized when Q is OH.

Another embodiment of this disclosure is realized when Z² is absent.

Another embodiment of this disclosure is realized when Z² is —CR^{b}R^{b}—. Another embodiment of this disclosure is realized when Z² is —CH₂CR^{b}R^{b}—. Another embodiment of this disclosure is realized when Z² is —CR^{b}R^{b}CH₂—.

Another embodiment of this disclosure is realized when each R^{b} is independently selected from hydrogen, OH, and CH₃. A subembodiment of this aspect of the disclosure is realized when one R^{b} is hydrogen and the other is OH. Another subembodiment of this aspect of the disclosure is realized when one R^{b} is hydrogen and the other is CH₃. A subembodiment of this aspect of the disclosure is realized when both R^{b} are CH₃. A subembodiment of this aspect of the disclosure is realized when both R^{b} are hydrogen.

Another embodiment of this disclosure is realized when both R^{b} combine to form a C₃₋₆ spirocycloalkyl. Another subembodiment of this aspect of the disclosure is realized when both R^{b} combine to form spirocyclopropyl.

Another embodiment of this disclosure is realized when Z¹ is —O—.

Another embodiment of this disclosure is realized when Z¹ is —NH—.

Another embodiment of this disclosure is realized when X is a linking group that is W1.

A subembodiment of this aspect of the disclosure is realized when W1 is

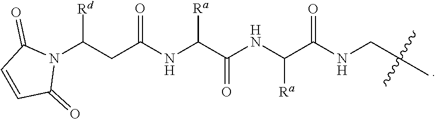

Another subembodiment of this aspect of the disclosure is realized when W1 is

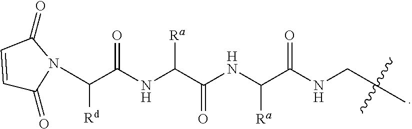

Another subembodiment of this aspect of the disclosure is realized when R^{d} of W1 is hydrogen. Another subembodiment of this aspect of the disclosure is realized when R^{d} of W1 is —CH₂NHC(O)X¹Q.

Another embodiment of this disclosure is realized when X is a linking group that is W2. A subembodiment of this aspect of the disclosure is realized when W2 is

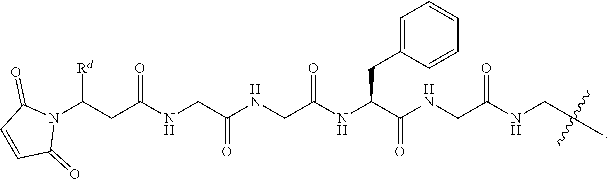

Another subembodiment of this aspect of the disclosure is realized when W2 is

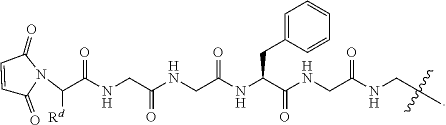

Another subembodiment of this aspect of the disclosure is realized when R^{d} of W2 is hydrogen. Another subembodiment of this aspect of the disclosure is realized when R^{d} of W2 is —CH₂NHC(O)X¹Q.

Another embodiment of this disclosure is realized when X is a linking group that is W3.

Another embodiment of this disclosure is realized when X is a linking group that is W4.

Another embodiment of this disclosure is realized when X is a linking group that is W5.

Another embodiment of this disclosure is realized when X is a linking group that is W6. A subembodiment of this aspect of the disclosure is realized when W6 is

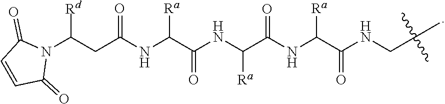

Another subembodiment of this aspect of the disclosure is realized when W6 is

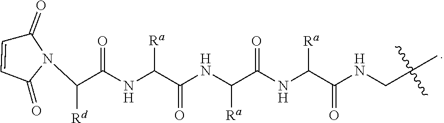

Another subembodiment of this aspect of the disclosure is realized when R^{d} of W6 is hydrogen. Another subembodiment of this aspect of the disclosure is realized when R^{d} of W6 is —CH₂NHC(O)X¹Q. Another embodiment of this disclosure is realized when X is a linking group that is W7. A subembodiment of this disclosure is realized when W7 is

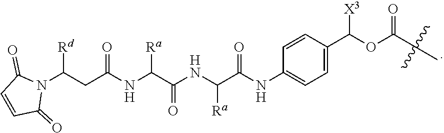

A subembodiment of this disclosure is realized when W7 is

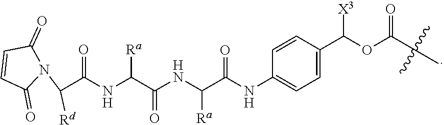

Another subembodiment of this aspect of the disclosure is realized when R^{d} of W7 is hydrogen. Another subembodiment of this aspect of the disclosure is realized when R^{d} of W7 is —CH₂NHC(O)X¹Q.

Another subembodiment of this aspect of the disclosure is realized when X³ is hydrogen or —C(O)NR^{a}R^{z}. Another subembodiment of this aspect of the disclosure is realized when X³ is hydrogen. Another subembodiment of this aspect of the disclosure is realized when X³ is —C(O)NR^{a}R^{z} wherein R^{a} is selected from hydrogen and C₁₋₆ alkyl, and R^{z} is X¹. Another subembodiment of this aspect of the disclosure is realized when X³ is —C(O)NR^{a}R^{z} and R^{a} and R^{z} combine to form a C₃₋₁₀ cycloalkyl or 3 to 10 membered heterocyclyl. An aspect of this subembodiment is realized when R^{a} and R^{z} combine to form cycloalkyl selected from cyclopropyl, cyclobutyl, cyclopentyl and cyclohexyl. Another aspect of this subembodiment is realized when the 3 to 10 membered heterocyclyl formed by R^{a} and R^{z} is a cyclic amine selected from piperidine, piperazine, azetidine, aziridine, pyrrolidine, azepane, morpholine, pyridine, and imidazole. A further embodiment of disclosure is realized when the cyclic amine is attached to the X³ carbonyl by its nitrogen atom. A further embodiment of this aspect of the invention is realized when the cyclic amine formed by R^{a} and R^{z} is piperidine. A further embodiment of this aspect of the invention is realized when the cyclic amine formed by R^{a} and R^{z} is piperazine. A further embodiment of this aspect of the invention is realized when the cyclic amine formed by R^{a} and R^{z} is azetidine. A further embodiment of this aspect of the invention is realized when the cyclic amine formed by R^{a} and R^{z} is aziridine. A further embodiment of this aspect of the invention is realized when the cyclic amine formed by R^{a} and R^{z} is pyrrolidine. A further embodiment of this aspect of the invention is realized when the cyclic amine formed by R^{a} and R^{z} is azepane. A further embodiment of this aspect of the invention is realized when the cyclic amine formed by R^{a} and R^{z} is pyridine. A further embodiment of this aspect of the invention is realized when the cyclic amine formed by R^{a} and R^{z} is imidazole. A further embodiment of this aspect of the invention is realized when the cyclic amine formed by R^{a} and R^{z} is

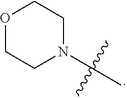 [SMILES: *N1CCOCC1]

Another embodiment of this disclosure is realized when X is a linking group that is W8. A subembodiment of this aspect of the disclosure is realized when X⁴ is hydrogen. A subembodiment of this aspect of the disclosure is realized when X⁴ is

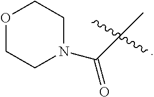 [SMILES: *C(=O)N1CCOCC1]

An embodiment of this disclosure is realized when each R^{a} is independently selected from hydrogen and methyl. Another embodiment of this disclosure is realized when R^{a} is methyl. Another embodiment of this disclosure is realized when R^{a} is hydrogen.

An embodiment of the disclosure of Formula I is represented by structural Formula II

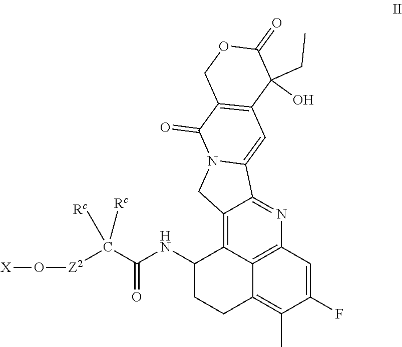

or a salt, pharmaceutically acceptable salt or solvate thereof, wherein X, Z², and R^{c} are as described herein. A subembodiment of the disclosure of Formula II is realized when each R^{c} is independently selected from hydrogen, OH, CH₃, CH₂OH, CHF₂, CH₂F, CF₃, fluorine, and chlorine. A subembodiment of the disclosure of Formula II is realized when one R^{c} is hydrogen and the other is OH. Another subembodiment of the disclosure of Formula II is realized when one R^{c} is hydrogen and the other is CH₃. Another subembodiment of the disclosure of Formula II is realized when one R^{c} is hydrogen and the other is CH₂OH. Another subembodiment of the disclosure of Formula II is realized when one R^{c} is CH₃ and the other is CHF₂. Another subembodiment of the disclosure of Formula II is realized when one R^{c} is hydrogen and the other is OH. A subembodiment of the disclosure of Formula II is realized when both R^{c} are CH₃. A subembodiment of the disclosure of Formula II is realized when both R^{c} are fluorine. A subembodiment of the disclosure of Formula II is realized when both R^{c} are hydrogen.

Another embodiment of the disclosure of Formula II is realized when Z² is —CR^{b}R^{b}. Another embodiment of the disclosure of Formula II is realized when Z² is —CH₂CR^{b}R^{b}. Another embodiment of the disclosure of Formula II is realized when Z² is —CR^{b}R^{b}CH₂—. A subembodiment of this aspect of the disclosure is realized when each R^{b} is independently selected from hydrogen, OH, and CH₃. A subembodiment of this aspect of the disclosure is realized when Z² is selected from —CH₂—, —CH₂CH₂—, —CH₂CH(CH₃)—, —CH₂CH(OH)—, and —CH₂— spirocyclopropyl-. A subembodiment of this aspect of the disclosure is realized when Z² is —CH₂—. A subembodiment of this aspect of the disclosure is realized when Z² is —CH₂CH₂—.

Another embodiment of the disclosure of Formula II is realized when X is a linking group that is W1. A subembodiment of Formula II is realized when W1 is

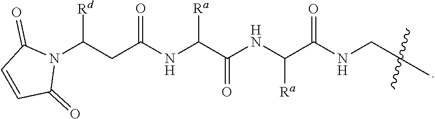

Another subembodiment of Formula II is realized when W1 is

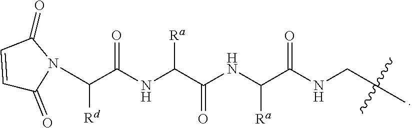

Another subembodiment of Formula II is realized when R^{d} of W1 is hydrogen. Another subembodiment of Formula II is realized when R^{d} of W1 is —CH₂NHC(O)X¹Q. An aspect of this embodiment is realized when X¹ is a PEG of 1 to 24 subunits. Another aspect of this embodiment is realized when X¹ is a PEG of 1 to 12 subunits.

Another embodiment of Formula II is realized when X is a linking group that is W2. A subembodiment of Formula II is realized when W2 is

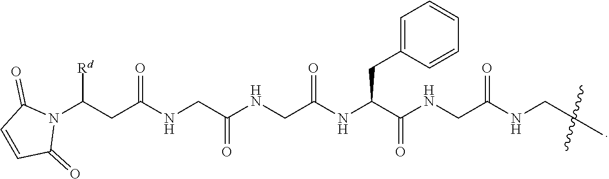

Another subembodiment Formula II is realized when W2 is

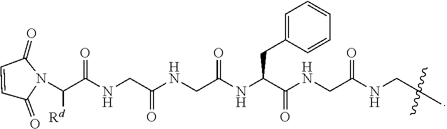

Another subembodiment of Formula II is realized when R^{d} of W2 is hydrogen. Another subembodiment of Formula II is realized when R^{d} of W2 is —CH₂NHC(O)X¹Q.

Another embodiment of Formula II is realized when X is a linking group that is W3.

Another embodiment of Formula II is realized when X is a linking group that is W4.

Another embodiment of Formula II is realized when X is a linking group that is W5.

Another embodiment of Formula II is realized when X is a linking group that is W6. A subembodiment of Formula II is realized when W6 is

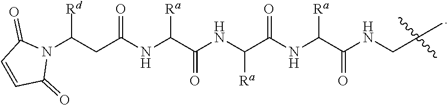

Another subembodiment of Formula II is realized when W6 is

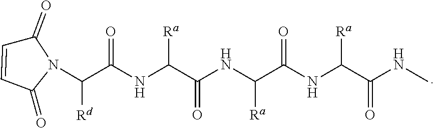

Another subembodiment of Formula II is realized when R^{d} of W6 is hydrogen. Another subembodiment of Formula II is realized when R^{d} of W6 is —CH₂NHC(O)X¹Q.

An embodiment of Formula II is realized when each R^{a} is independently selected from hydrogen and methyl. Another embodiment of Formula II is realized when R^{a} is methyl. Another embodiment of Formula II is realized when R^{a} is hydrogen.

An embodiment of the disclosure of Formula I is represented by structural Formula III:

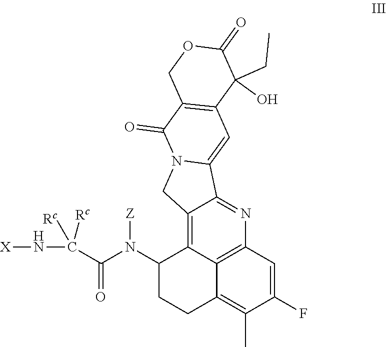

or a salt, pharmaceutically acceptable salt or solvate thereof, wherein X, Z, and R^{c} are as described herein. A subembodiment of the disclosure of Formula III is realized when each R^{c} is independently selected from hydrogen, OH, CH₃, CH₂OH, CHF₂, CH₂F, CF₃, fluorine, and chlorine. A subembodiment of the disclosure of Formula III is realized when one R^{c} is hydrogen and the other is OH. Another subembodiment of the disclosure of Formula III is realized when one R^{c} is hydrogen and the other is CH₃. Another subembodiment of the disclosure of Formula III is realized when one R^{c} is hydrogen and the other is CH₂OH. Another subembodiment of the disclosure of Formula III is realized when one R^{c} is CH₃ and the other is CHF₂. Another subembodiment of the disclosure of Formula III is realized when one R^{c} is hydrogen and the other is OH. A subembodiment of the disclosure of Formula III is realized when both R^{c} are CH₃. A subembodiment of the disclosure of Formula III is realized when both R^{c} are fluorine. A subembodiment of the disclosure of Formula III is realized when both R^{c} are hydrogen.

Another embodiment of the disclosure of Formula III is realized when Z is hydrogen.

Another embodiment of the disclosure of Formula III is realized when Z is —CH₂C(R^{x})(R^{y})CHF₂. A subembodiment of this aspect of the disclosure is realized when Z is CH₂C(F₂)CHF₂.

Another embodiment of Formula III is realized when X is a linking group that is W2. A subembodiment of Formula III is realized when W2 is

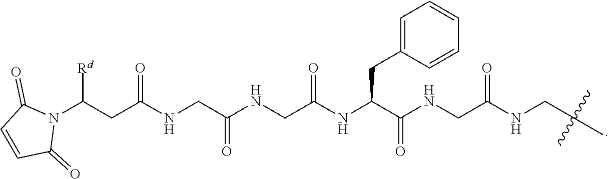

Another subembodiment Formula III is realized when W2 is

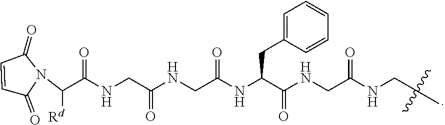

Another subembodiment of Formula III is realized when R^{d} of W2 is hydrogen. Another subembodiment of Formula III is realized when R^{d} of W2 is —CH₂NHC(O)X¹Q.

Another embodiment of Formula III is realized when X is a linking group that is W3.

Another embodiment of Formula III is realized when X is a linking group that is W4.

Another embodiment of Formula III is realized when X is a linking group that is W5.

Another embodiment of Formula III is realized when X is a linking group that is W6. A subembodiment of Formula III is realized when W6 is

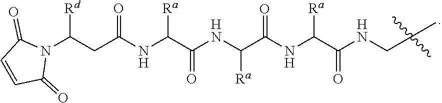

Another subembodiment of Formula III is realized when W6 is

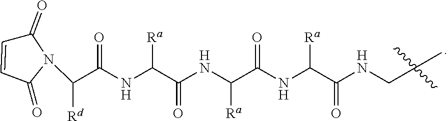

Another subembodiment of Formula III is realized when R^{d} of W6 is hydrogen. Another subembodiment of Formula III is realized when R^{d} of W6 is —CH₂NHC(O)X¹Q.

An embodiment of Formula III is realized when each R^{a} is independently selected from hydrogen and methyl. Another embodiment of Formula III is realized when R^{a} is methyl. Another embodiment of Formula III is realized when R^{a} is hydrogen.

Another embodiment of the disclosure of Formula III is realized when X is a linking group that is W7. A subembodiment of Formula III is realized when W7 is

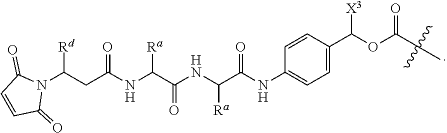

A subembodiment of Formula III is realized when W7 is

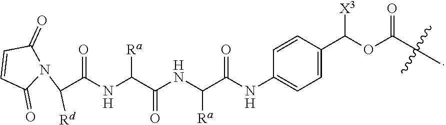

Another subembodiment of Formula III is realized when R^{d} of W7 is hydrogen. Another subembodiment of Formula III is realized when R^{d} of W7 is —CH₂NHC(O)X¹Q. Another subembodiment of Formula III is realized when X³ is hydrogen or —C(O)NR^{a}R^{z}. Another subembodiment of Formula III the disclosure is realized when X³ is hydrogen. Another subembodiment of this aspect of Formula III is realized when X³ is —C(O)NR^{a}R^{z} wherein R^{a} is selected from hydrogen and C₁₋₆ alkyl, and R^{z} is X¹. Another subembodiment of Formula III is realized when X³ is —C(O)NR^{a}R^{z} and R^{a} and R^{z} combine to form a C₃₋₁₀ cycloalkyl or a 3 to 10 membered heterocyclyl. An aspect of this subembodiment is realized when R^{a} and R^{z} combine to form cycloalkyl selected from cyclopropyl, cyclobutyl, cyclopentyl and cyclohexyl. Another aspect of this subembodiment is realized when 3 to 10 membered heterocyclyl formed by R^{a} and R^{z} is a cyclic amine selected from piperidine, piperazine, azetidine, aziridine, pyrrolidine, azepane, morpholine, pyridine, and imidazole. A further embodiment of disclosure is realized when the cyclic amine is attached to the X³ carbonyl by its nitrogen atom. A further embodiment of this aspect of the invention is realized when the cyclic amine formed by R^{a} and R^{z} is piperidine. A further embodiment of this aspect of the invention is realized when the cyclic amine formed by R^{a} and R^{z} is piperazine. A further embodiment of this aspect of the invention is realized when the cyclic amine formed by R^{a} and R^{z} is azetidine. A further embodiment of this aspect of the invention is realized when the cyclic amine formed by R^{a} and R^{z} is aziridine. A further embodiment of this aspect of the invention is realized when the cyclic amine formed by R^{a} and R^{z} is pyrrolidine. A further embodiment of this aspect of the invention is realized when the cyclic amine formed by R^{a} and R^{z} is azepane. A further embodiment of this aspect of the invention is realized when the cyclic amine formed by R^{a} and R^{z} is pyridine. A further embodiment of this aspect of the invention is realized when the cyclic amine formed by R^{a} and R^{z} is imidazole. A further embodiment of this aspect of the invention is realized when the cyclic amine formed by R^{a} and R^{z} is

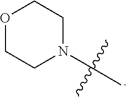 [SMILES: *N1CCOCC1]

Another embodiment of Formula III is realized when X is a linking group that is W8. A subembodiment of Formula III is realized when X is a linking group that is W8 and X⁴ is hydrogen. A subembodiment of Formula III is realized when X is a linking group that is W8 and X⁴ is

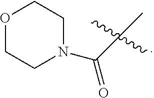 [SMILES: *C(=O)N1CCOCC1]

An embodiment of Formula III is realized when each R^{a} is independently selected from hydrogen and methyl. Another embodiment of Formula III is realized when R^{a} is methyl. Another embodiment of Formula III is realized when R^{a} is hydrogen.

In another embodiment, it may be desirable to synthesize the linker prior to conjugation to the drug or targeting moiety to yield ADCs. In such embodiments, the linker compounds may act as intermediate compounds. Intermediates of this disclosure are represented by the compound of Formula V:

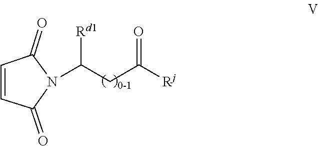

wherein:
R^{j} is selected from OH, —NH₂, —NHR^{k}R^{g}, —NHR^{k}NH(CH₂)ₙOR^{q}, —NHR^{k}NH(CH₂)ₙOC(O)CH₃, —NHX^{1a}(CH₂)ₙC(O)R^{k}NHCH₂OC(O)CH₃, —NHR^{k}NHR^{L}, —NHX^{1a}R^{k}NHR^{L}, and —NHCH₂O(CH₂)ₙCH(OH)C(O)OH;
R^{g} is C(O)OH or C(O)NH₂;
R^{k} is an amino acid residue of up to 10 amino acids;
R^{L} is selected from:

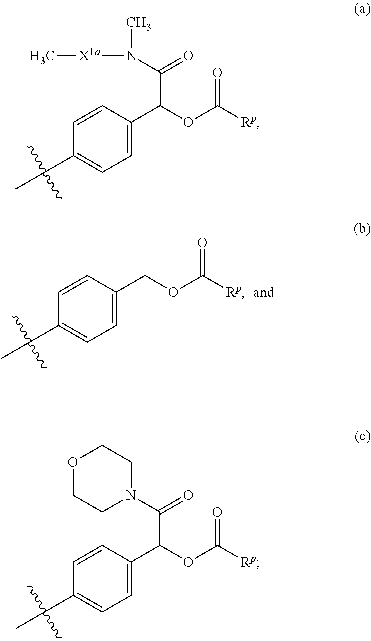

R^{p} is selected from NH₂ and

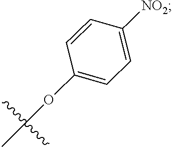 [SMILES: *Oc1ccc([N+](=O)[O-])cc1]

R^{q} is hydrogen or C₁₋₆ alkyl;
X^{1a} is a PEG of 1 to 24 —CH₂CH₂O— subunits;
R^{d1} is hydrogen, —CH₂NHC(O)X^{1a}Q or —CH₂NHC(O)X²¹Q;
Q is C₁₋₆ alkyl or H;
X²¹ is selected from a PEG of 1 to 24 —CH₂CH₂O— subunits, PEG-amino sugar, and p-aminobenzylcarbonyl and
n is 1, 2, 3, or 4.

An embodiment of Formula V is realized when R^{k} is an amino acid residue of up to 10 amino acids. Another subembodiment of this aspect of Formula V is realized when R^{k} is selected from 1 to 8, 1 to 6, 1 to 4, 1 to 2, 2 to 8 and 2 to 6 amino acid residues. Another subembodiment of Formula V is realized when the amino acid residues of R^{k} are derived from one or more of the same or different amino acids selected from glycine, alanine, phenylalanine, valine, lysine, citrulline, and sarcosine. Another subembodiment of Formula V is realized when the amino acid residues of R^{k} are selected from glucamine, glucosamine, and galactosamine. Another subembodiment of Formula V is realized when the amino acid residue of R^{k} is glucamine. Another subembodiment of Formula V is realized when the amino acid residue of R^{k} is glucosamine. Another subembodiment of Formula V is realized when the amino acid residue of R^{k} is galactosamine.

An embodiment of Formula V is realized when R^{j} is OH.

An embodiment of Formula V is realized when R^{j} is NH₂.

An embodiment of Formula V is realized when R^{j} is —NHR^{k}R^{g}

An embodiment of Formula V is realized when R^{j} is —NHR^{k}NH(CH₂)ₙOR^{q}. A subembodiment of this aspect of the disclosure is realized when R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine. Another subembodiment of this aspect of the disclosure is realized when R^{q} is hydrogen. Another subembodiment of this aspect of the disclosure is realized when R^{q} is C₁₋₆ alkyl. Another subembodiment of this aspect of the disclosure is realized when R^{q} is methyl, ethyl, or propyl.

An embodiment of Formula V is realized when R^{j} is —NHR^{k}NHCH₂OC(O)CH₃. A subembodiment of this aspect of the disclosure is realized when R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine.

An embodiment of Formula V is realized when R^{j} is —NHX^{1a}(CH₂)₂C(O)R^{k}NHCH₂OC(O)CH₃. A subembodiment of this aspect of the disclosure is realized when R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine. Another subembodiment of this aspect of the disclosure is realized when X^{1a} is a PEG of 1 to 24 subunits. Another aspect of this embodiment is realized when X^{1a} is a PEG of 1 to 12 subunits.

An embodiment of Formula V is realized when R^{j} is —NHR^{k}NHR^{L}. A subembodiment of this aspect of the disclosure is realized when R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine. Another subembodiment of this aspect of the disclosure is realized when R^{L} is (a). Another subembodiment of this aspect of the disclosure is realized when R^{L} is (b). Another subembodiment of this aspect of the disclosure is realized when R^{L} is (c). Another subembodiment of this aspect of the disclosure is realized when R^{p} is NH₂ when R^{L} is (a), (b), or (c). Another subembodiment of this aspect of the disclosure is realized when R^{p} is

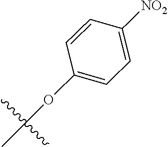 [SMILES: *Oc1ccc([N+](=O)[O-])cc1]

when R^{L} is (a), (b), or (c).

An embodiment of Formula V is realized when R^{j} is —NHX^{1a}R^{k}NHR^{L}. A subembodiment of this aspect of the disclosure is realized when R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine. Another subembodiment of this aspect of the disclosure is realized when R^{L} is (a). Another subembodiment of this aspect of the disclosure is realized when R^{L} is (b). Another subembodiment of this aspect of the disclosure is realized when R^{L} is (c). Another subembodiment of this aspect of the disclosure is realized when R^{p} is NH₂ when R^{L} is (a), (b), or (c). Another subembodiment of this aspect of the disclosure is realized when R^{p} is

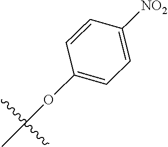 [SMILES: *Oc1ccc([N+](=O)[O-])cc1]

when R^{L} is (a), (b), or (c). Another subembodiment of this aspect of the disclosure is realized when X^{1a} is a PEG of 1 to 24 subunits. Another aspect of this embodiment is realized when X^{1a} is a PEG of 1 to 12 subunits.

An embodiment of Formula V is realized when R^{j} is —NHCH₂O(CH₂)₂CH(OH)C(O)OH,

Another embodiment of Formula V is realized when R^{j} is —NHR^{k}H and R^{k} is an amino acid residue of up to 10 amino acids. A subembodiment of this aspect of the disclosure is realized when amino acid residues R^{k} are derived from one or more of the same or different amino acids selected from glycine, alanine, phenylalanine, valine, lysine, citrulline, and sarcosine. Another subembodiment of this aspect of the disclosure is realized when the amino acid residues of R^{k} are selected from glucamine, glucosamine, and galactosamine. Another subembodiment of this aspect of the disclosure is realized when the amino acid residue of R^{k} is glucamine. Another subembodiment of this aspect of the disclosure is realized when the amino acid residue of R^{k} is glucosamine. Another subembodiment of this aspect of the disclosure is realized when the amino acid residue of R^{k} is galactosamine.

Another embodiment of Formula V is realized when R^{d1} is hydrogen.

Another embodiment of Formula V is realized when R^{d1} is CH₂NHC(O)X^{1a}Q, wherein X1a and Q are as described herein.

Another embodiment of Formula V is realized when R^{d1} is —CH₂NHC(O)X²¹Q; wherein X²¹ is a PEG of 1 to 24 —CH₂CH₂O— subunits or PEG-amino sugar, wherein the PEG in the PEG-amino sugar is a polyethylene glycol of 1 to 24 —CH₂CH₂O— subunits. A subembodiment of this aspect of the disclosure is realized when the PEG is 4 to 12 —CH₂CH₂O— subunits. Another subembodiment of this aspect of the disclosure is realized when the PEG is 4 —CH₂CH₂O— subunits. Another subembodiment of this aspect of the disclosure is realized when the PEG is 6 —CH₂CH₂O— subunits. Another subembodiment of this aspect of the disclosure is realized when the PEG is 8 —CH₂CH₂O— subunits. Another subembodiment of this aspect of the disclosure is realized when the PEG is 10 —CH₂CH₂O— subunits. Another subembodiment of this aspect of the disclosure is realized when the PEG is 12 —CH₂CH₂O— subunits.

Another embodiment of Formula V is realized when the amino sugar of PEG-amino sugar is an open chain sugar derived amino alcohol or glycamine wherein the sugar is selected from glucose, galactose, sorbital, mannitol, xylitol, arabitol, ribitol, glycerol, ethylene glycol, galactitol and the like. A subembodiment of this aspect of Formula V is realized when the amino sugar of PEG-amino sugar is a closed chain amino alcohol selected from glucosamine, glucamine, galactosamine, and the like. A non-limiting example of an amino-sugar is represented by diamino sugar Si:

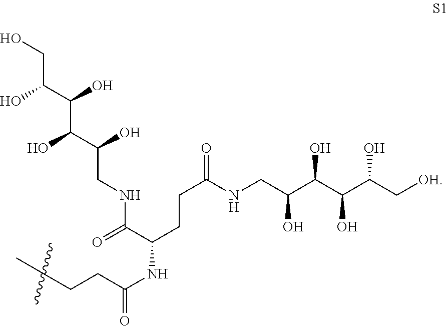 [SMILES: *CCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO]

Another embodiment of Formula V is realized when Q is C₁₋₆ alkyl selected from methyl, ethyl, propyl, butyl or hexyl. A subembodiment of this aspect of the disclosure is realized when Q is methyl.

Another embodiment of Formula V is realized when Q is H.

Exemplary intermediate linker compounds of the present disclosure, or a salt thereof are described herein. An embodiment of this disclosure is realized when the linker compounds are selected from Table 1:

TABLE 1
Structure

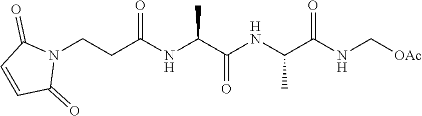 [SMILES: CC(=O)OCNC(=O)[C@H](C)NC(=O)[C@H](C)NC(=O)CCN1C(=O)C=CC1=O]

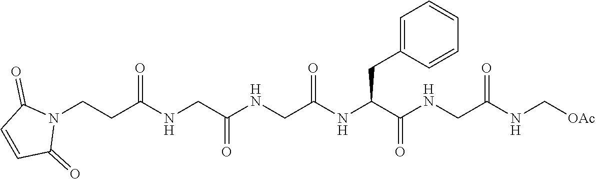 [SMILES: CC(=O)OCNC(=O)CNC(=O)[C@H](Cc1ccccc1)NC(=O)CNC(=O)CNC(=O)CCN1C(=O)C=CC1=O]

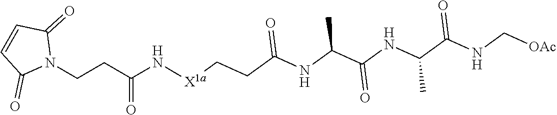

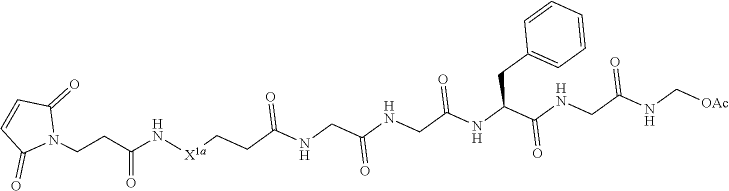

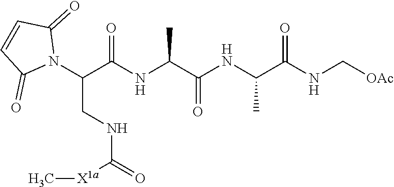

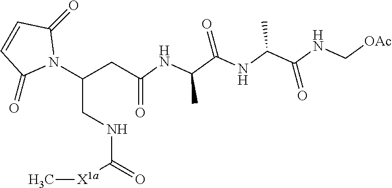

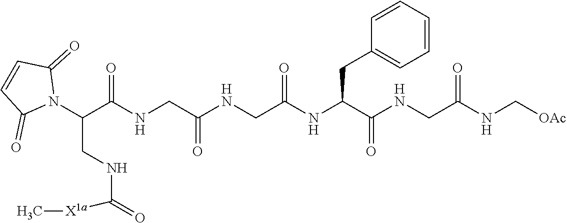

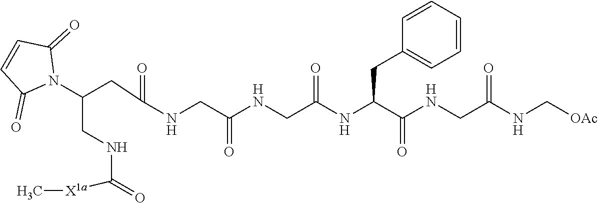

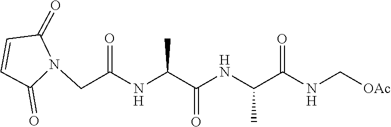 [SMILES: CC(=O)OCNC(=O)[C@H](C)NC(=O)[C@H](C)NC(=O)CN1C(=O)C=CC1=O]

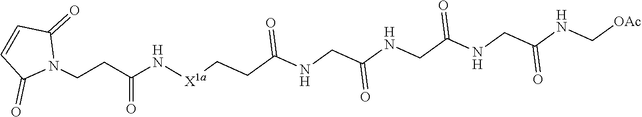

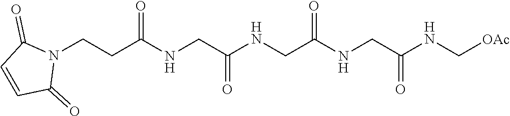 [SMILES: CC(=O)OCNC(=O)CNC(=O)CNC(=O)CNC(=O)CCN1C(=O)C=CC1=O]

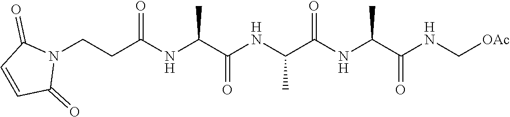 [SMILES: CC(=O)OCNC(=O)[C@H](C)NC(=O)[C@H](C)NC(=O)[C@H](C)NC(=O)CCN1C(=O)C=CC1=O]

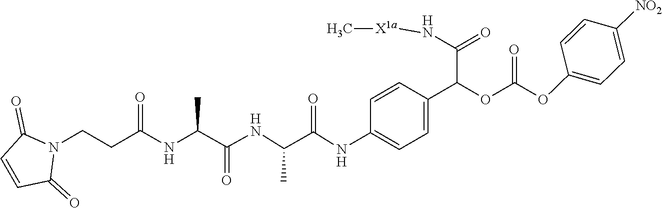

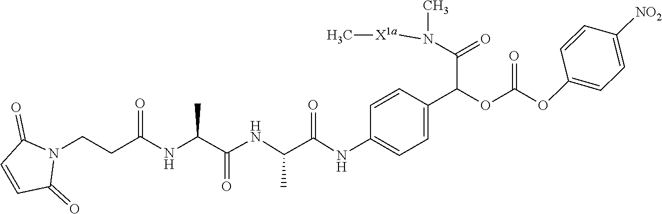

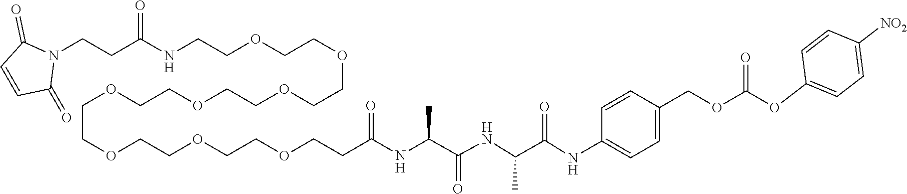 [SMILES: C[C@H](NC(=O)CCOCCOCCOCCOCCOCCOCCOCCOCCNC(=O)CCN1C(=O)C=CC1=O)C(=O)N[C@@H](C)C(=O)Nc1ccc(COC(=O)Oc2ccc([N+](=O)[O-])cc2)cc1]

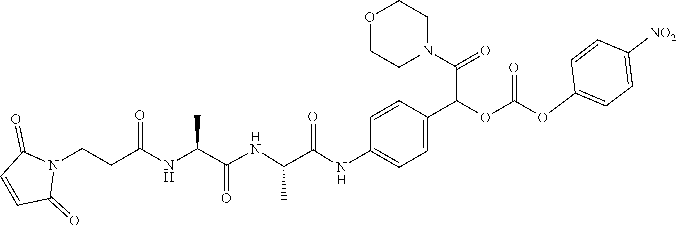 [SMILES: C[C@H](NC(=O)CCN1C(=O)C=CC1=O)C(=O)N[C@@H](C)C(=O)Nc1ccc(C(OC(=O)Oc2ccc([N+](=O)[O-])cc2)C(=O)N2CCOCC2)cc1]

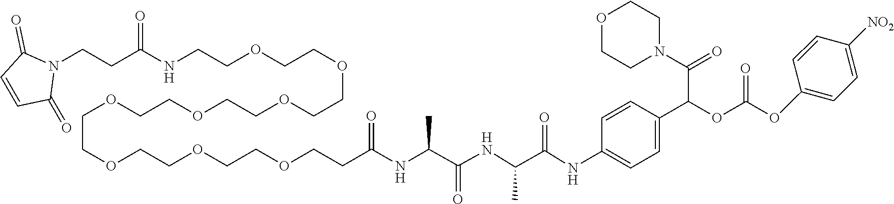 [SMILES: C[C@H](NC(=O)CCOCCOCCOCCOCCOCCOCCOCCOCCNC(=O)CCN1C(=O)C=CC1=O)C(=O)N[C@@H](C)C(=O)Nc1ccc(C(OC(=O)Oc2ccc([N+](=O)[O-])cc2)C(=O)N2CCOCC2)cc1]

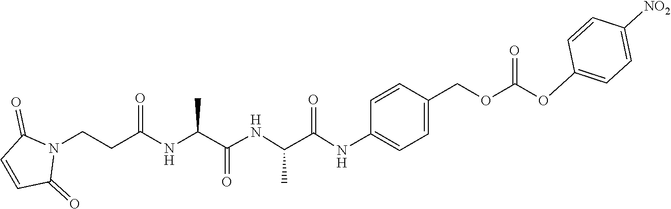 [SMILES: C[C@H](NC(=O)CCN1C(=O)C=CC1=O)C(=O)N[C@@H](C)C(=O)Nc1ccc(COC(=O)Oc2ccc([N+](=O)[O-])cc2)cc1]

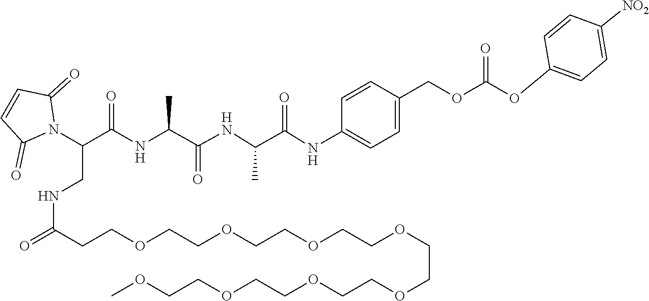 [SMILES: COCCOCCOCCOCCOCCOCCOCCOCCC(=O)NCC(C(=O)N[C@@H](C)C(=O)N[C@@H](C)C(=O)Nc1ccc(COC(=O)Oc2ccc([N+](=O)[O-])cc2)cc1)N1C(=O)C=CC1=O]

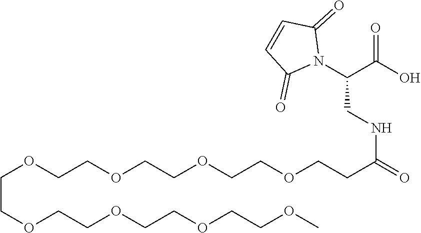 [SMILES: COCCOCCOCCOCCOCCOCCOCCOCCC(=O)NC[C@@H](C(=O)O)N1C(=O)C=CC1=O]

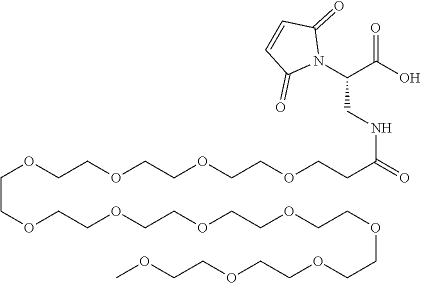 [SMILES: COCCOCCOCCOCCOCCOCCOCCOCCOCCOCCOCCOCCC(=O)NC[C@@H](C(=O)O)N1C(=O)C=CC1=O]

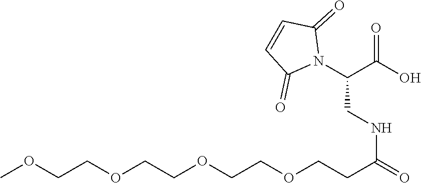 [SMILES: COCCOCCOCCOCCC(=O)NC[C@@H](C(=O)O)N1C(=O)C=CC1=O]

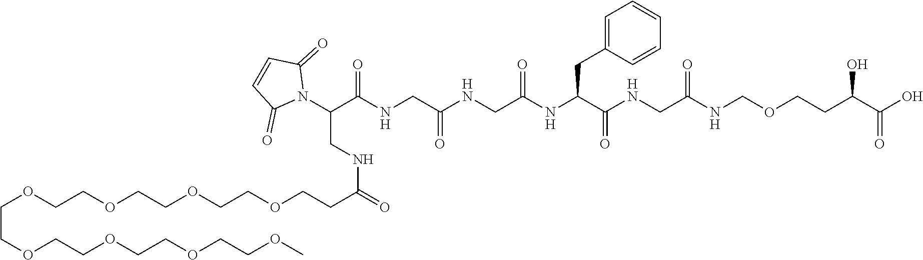 [SMILES: COCCOCCOCCOCCOCCOCCOCCOCCC(=O)NCC(C(=O)NCC(=O)NCC(=O)N[C@@H](Cc1ccccc1)C(=O)NCC(=O)NCOCC[C@@H](O)C(=O)O)N1C(=O)C=CC1=O]

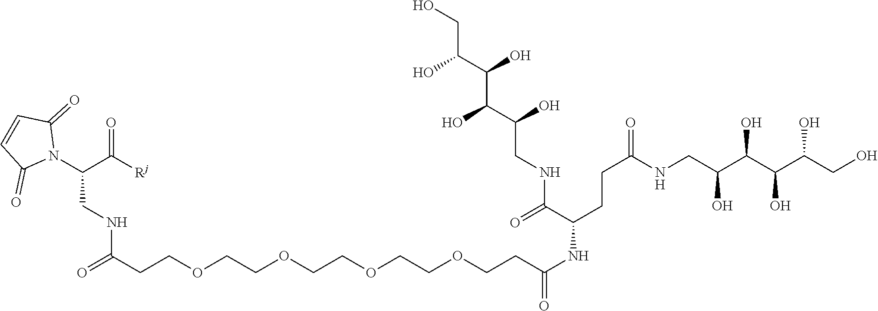

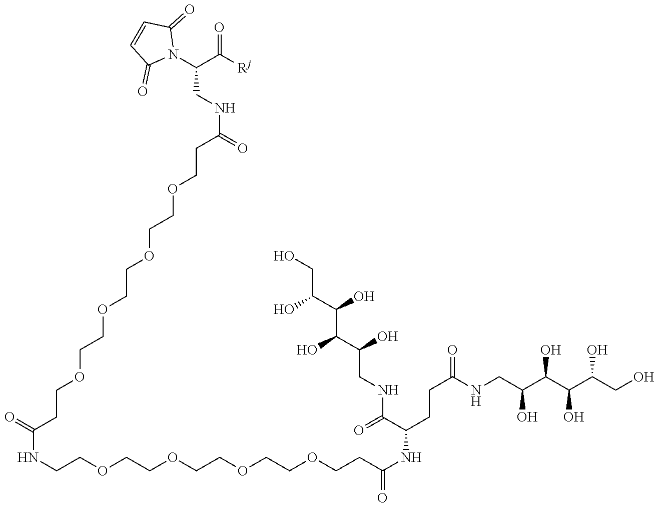

or salts, including pharmaceutically acceptable salts, solvates, or stereoisomers thereof, wherein X^{1a} is a PEG of 1 to 24, 4 to 12, 4, 6, 8, 10, or 12 —CH₂CH₂O— subunits and R^{j} is as described herein.

The compounds of the present disclosure have utility for conjugation to antibodies or other targeting moieties to generate antibody-drug conjugates (ADCs), or other targeting ligand conjugates, for oncology indications. Thus, an embodiment of the present disclosure is represented by ADCs of structural Formula IV:

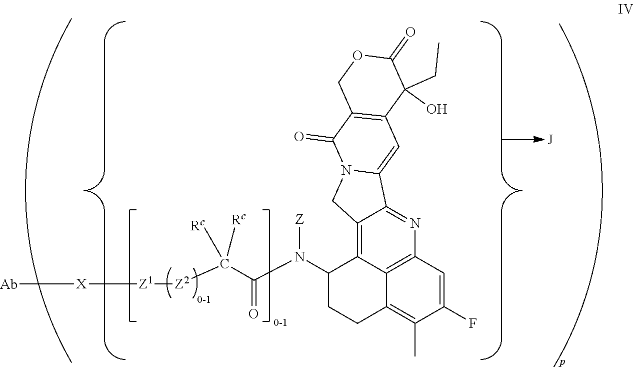

wherein Z, R^{c}, Z², Z¹, and X are as described herein, p denotes the number of drug linker moieties conjugated to a targeting moiety, (e.g., antibody). The average number of drug linker moieties in a Ligand-Drug Conjugate composition is the drug antibody ratio (DAR) and Ab is a ligand, such as an antibody (Ab) or other targeting moiety. X is the maleimide containing linker structure attached to a peptide linker that terminates with a hemiaminal or PABC connection to the camptothecin-derived-payload J. In some embodiments, p is an integer selected from 1, 2, 3, 4, 5, 6, 7, 8, 9, 10, 11, 12, 13, 14, 15, 16, 17, 18, 19, 20, 21, 22, 23, and 24. In some embodiments, p is an integer from 1 to 24, 1 to 12, or 1 to 8, or is 4 or 8. In some embodiments, p is 2, 4, 6, or 8.
The linker-payload can be conjugated to cysteine residues in various ligand conjugates such as an antibody to yield antibody drug conjugates (ADCs). The cysteine residue of the antibody forms a bond with the reactive maleimide on the linker group.

The ligand can be any moiety with a free cysteine group including, but not limited to, antibodies, proteins, peptides, polypeptides, or engineered antibodies modified to provide a free cysteine. An aspect of this is realized when the ligand is an antibody, preferably an intact antibody. The Ligand acts to target and present the drug to the particular target cell population with which the ligand interacts. Suitable Ligand include, for example, antibodies, e.g., full-length antibodies and antigen binding fragments thereof, interferons, lymphokines, hormones, growth factors and colony-stimulating factors, vitamins, nutrient transport molecules (such as, but not limited to, transferrin), or any other cell binding molecule or substance, including small molecules and peptides. The ligand can be, for example, a non-antibody protein targeting agent.

When the conjugates comprise non-immunoreactive protein, polypeptide, or peptide Ligands instead of an antibody, useful non-immunoreactive protein, polypeptide, or peptide Ligands include, but are not limited to, transferrin, epidermal growth factors (“EGF”), bombesin, gastrin, gastrin releasing peptide, platelet-derived growth factor, IL-2, IL-6, transforming growth factors (“TGF”), such as TGF-α and TGF-β, vaccinia growth factor (“VGF”), insulin and insulinlike growth factors I and II, somatostatin, lectins and apoprotein from low density lipoprotein.

Particularly preferred ligands are antibodies, including intact antibodies. In fact, in any of the embodiments described herein, the ligand can be an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187 each of which is incorporated herein by reference in its entirety.

Completely human antibodies are particularly desirable and can be produced using transgenic mice that are incapable of expressing endogenous immunoglobulin heavy and light chains genes, but which can express human heavy and light chain genes. Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivatization by known protecting/blocking groups, proteolytic cleavage, linkage to a cellular antibody or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

In a specific embodiment, known antibodies for the treatment of cancer can be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing.

In another specific embodiment, antibodies for the treatment of an autoimmune disease are used in accordance with the compositions and methods of the disclosure. Antibodies immunospecific for an antigen of a cell that is responsible for producing autoimmune antibodies can be obtained from any organization (e.g., a university scientist or a company) or produced by any method known to one of skill in the art such as, e.g., chemical synthesis or recombinant expression techniques.

In another embodiment, it may be desirable to conjugate components of the linker to the ligand (e.g., antibody) prior to attaching the camptothecin-derived drug component of the ADC. For example, in embodiments where a thiol containing substituent, e.g., cysteine, is being used to attach the camptothecin-derived drug component, it may be desirable to conjugate components of the linker to the ligand (e.g., antibody) prior to attaching the camptothecin-derived drug component of the ADC.

An aspect of this disclosure relates to a composition or pharmaceutical composition comprising a compound of Formulae I, II, III, IV, V or a salt, pharmaceutically acceptable salt or solvate thereof and one or more pharmaceutically acceptable carrier(s), diluent(s) or excipients(s).

Another aspect of the disclosure relates to a composition or pharmaceutical composition comprising a compound of Formulae I, II, III, IV, or V as described herein, or a tautomer, mesomere, racemate, enantiomer, diastereomer thereof, or mixture thereof, or a salt, or pharmaceutically acceptable salt thereof and one or more pharmaceutically acceptable carrier(s), diluent(s) or excipient(s).

Another aspect of the disclosure relates to a compound of Formulae I, II, III, IV, V as described herein, or a tautomer, mesomere, racemate, enantiomer, diastereomer thereof, or mixture thereof, or a pharmaceutically acceptable salt thereof for use as a drug or drug component.

Another aspect of the disclosure relates to a compound of Formulae I, II, III, IV, V as described herein, or a tautomer, mesomere, racemate, enantiomer, diastereomer thereof, or mixture thereof, or a pharmaceutically acceptable salt thereof, or a pharmaceutical composition in the preparation of a medicament for treating or preventing a tumor.

Another aspect of the disclosure relates to intermediate linker compounds and compositions containing the same. Examples of intermediate linker compounds are represented by W1, W2, W3, W4, W5, W6, W7, W8 of X as described herein.

In another embodiment, the compounds of the disclosure include those identified herein as Examples in the tables below, and pharmaceutically acceptable salts thereof.

The compounds of the disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all the possible optical isomers and diastereomers in mixtures and as pure or partially purified compounds are included within the ambit of this disclosure. Unless a specific stereochemistry is indicated, the present disclosure is meant to encompass all such isomeric forms of these compounds.

The independent syntheses of these diastereomers or their chromatographic separations may be achieved as known in the art by appropriate modification of the methodology disclosed herein. Their absolute stereochemistry may be determined, amongst other methods, by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formulae I, II, III, IV, or V the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure may include all suitable isotopic variations of the compounds of generic Formulae I, II, III, IV, V. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or

may provide a compound useful as a standard for characterization of biological samples. For purposes of this disclosure when a compound is said to be “not deuterated” it means not enriched in deuterium beyond the background state. Isotopically-enriched compounds within generic Formulae I, II, III, IV, or V can be prepared without undue experimentation by conventional techniques well known to those skilled in the art or by processes analogous to those described in the Schemes and Examples herein using appropriate isotopically-enriched reagents and/or intermediates.

When a compound of the disclosure can form tautomers, all such tautomeric forms are also included within the scope of the present disclosure. For example, compounds including carbonyl —CH₂C(O)— groups (keto forms) may undergo tautomerism to form hydroxyl —CH═C(OH)— groups (enol forms). Both keto and enol forms, where present, are included within the scope of the present disclosure.

When any variable (e.g., R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant disclosure in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant disclosure can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-II. Accordingly, the methyl group of Illus-I is emerging from the page of the paper and the ethyl group in Illus-II is descending into the page, where the cyclohexene ring resides within the plane of the paper. It is assumed that the hydrogen on the same carbon as the methyl group of Illus-I descends into the page and the hydrogen on the same carbon as the ethyl group of Illus-II emerges from the page. The convention is the same where both a hashed and solid rectangle are appended to the same carbon as in Illus-III, the methyl group is emerging from the plane of the paper and the ethyl group is descending into the plane of the paper with the cyclohexene ring in the plane of the paper.

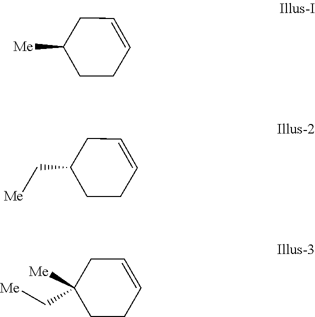 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”;

“Effective amount” or “therapeutically effective amount” is meant to describe the provision of an amount of at least one compound of the disclosure or of a composition comprising at least one compound of the disclosure which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of compound of Formula IV, that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;

“Patient” and “subject” means an animal, such as a mammal (e.g., a human being) and is preferably a human being;

“Prodrug” means compounds that are rapidly transformed, for example, by hydrolysis in blood, in vivo to the parent compound, e.g., conversion of a prodrug of Formula IV or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this disclosure includes prodrugs of the novel compounds of this disclosure;

The term “substituted” means that one or more of the enumerated substituents can occupy one or more of the bonding positions on the substrate typically occupied by “—H”, provided that such substitution does not exceed the normal valency rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution of a moiety is described (e.g., “optionally substituted”) the term means that if substituents are present, one or more of the enumerated substituents for the specified substrate can be present on the substrate in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the disclosure or a substituent appended to a variable portion of a structural representation of a group of compounds of the disclosure have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

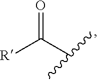

equally, with no differentiation implied using any or all of these representations;

The PEG provided herein comprises one or multiple polyethylene glycol chains containing repeating —CH₂CH₂O— subunits. The polyethylene glycol chains can be linked together, for example, in a linear, branched or star shaped configuration. Another embodiment of the disclosure is realized when the PEG Unit comprises at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. A PEG moiety having 4 repeating —CH2CH2O— can be referred to as -PEG4-, and similarly a PEG moiety having 8 repeating —CH2CH2O— units can be referred to as -PEG8-.

The term “antibody” as used herein is used in the broadest sense and specifically covers intact monoclonal antibodies, polyclonal antibodies, monospecific antibodies, multispecific antibodies (e.g., bispecific antibodies), and antibody fragments that exhibit the desired biological activity provided that the antibody fragment have the requisite number of attachment sites for a drug-linker. The native form of an antibody is a tetramer and consists of two identical pairs of immunoglobulin chains, each pair having one light chain and one heavy chain. In each pair, the light and heavy chain variable regions (VL and VH) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system, (see, e.g., Janeway et al., 2001, Immuno. Biology, 5th Ed., Garland Publishing, New York). An antibody can be of any type (e.g., IgG, IgE, IgM, IgD, and IgA), class (e.g., IgG1, IgG2, IgG3, IgG4, IgA1 and IgA2) or subclass. The antibody can be derived from any suitable species. In some aspects, the antibody is of human or murine origin. An antibody can be, for example, human, humanized or chimeric.

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀-alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates an alkyl moiety connecting a hydroxyl moiety to a substrate.

As used herein, when the term “alkyl” is modified by “substituted” or “optionally substituted”, it means that one or more C—H bonds in the alkyl moiety group is substituted, or optionally may be substituted, by a substituent bonded to the alkyl substrate which is called out in defining the moiety.

The term “cycloalkyl” means a moiety having a main hydrocarbon chain forming a mono- or bicyclo-cyclic aliphatic moiety comprising at least 3 carbon atoms (the minimum number necessary to provide a monocyclic moiety) up to the maximum number of specified carbon atoms, generally 8 for a monocyclic moiety and 10 for a bicyclic moiety, inclusive of spirocyclic moieties. Examples of cycloalkyl moieties include, but are not limited to, cyclopropyl, cyclobutyl, cyclopentyl or cyclohexyl. The term “cycloalkyl” also includes non-aromatic, fused multicyclic ring system comprising up to 20 carbon atoms which may optionally be substituted as defined herein for “alkyl” generally. Suitable multicyclic cycloalkyls are, for example, but are not limited to: 1-decalin; norbornyl; adamantly; and the like;

As used herein, the term “alkylene” refers to a saturated linear or branched aliphatic hydrocarbon group having two residues derived from the removal of two hydrogen atoms from the same carbon atom or two different carbon atoms of the parent alkane. The alkylene is a linear or branched group having 1 to 20 carbon atoms, preferably 1 to 12 carbon atoms, and more preferably 1 to 6 carbon atoms. Non-limiting examples are methylene, ethylene, propylene, butylene, pentylene, and the like.

Where a structural formula represents bonding between a moiety and a substrate using a bonding line that terminates in the middle of the structure, for example the following representations:

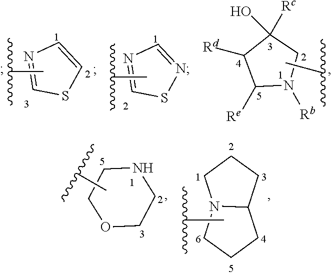

whether or not numbered the structure indicates that unless otherwise defined the moiety may be bonded to the substrate through any of available ring atom, for example, the numbered atoms of the example moieties.

The term “aryl” refers to a 6 to 14 membered all-carbon monocyclic ring or polycyclic fused ring (i.e., each ring in the system shares an adjacent pair of carbon atoms with another ring in the system) having a conjugated it-electron system, preferably a 6 to 10 membered aryl, for example, phenyl and naphthyl, and preferably phenyl.

The term “heteroaryl” refers to an aromatic 5-8 membered monocyclic, 8-12 membered bicyclic, or 11-14 membered tricyclic ring system having 1-3 heteroatoms for monocyclic, 1-6 heteroatoms for bicyclic, or 1-9 heteroatoms for tricyclic, said heteroatoms selected from O, N, or S (e.g., carbon atoms and 1-3, 1-6, or 1-9 heteroatoms of N, O, or S for monocyclic, bicyclic, or tricyclic, respectively). Non-limiting examples of heteroaryls are pyridyl, pyrazolyl, pyrimidinyl, furanyl, oxazolyl, triazolyl, oxadiazolyl, and thiophenyl. The heteroaryl groups herein described may also contain fused rings that share a common carbon-carbon bond.

The term “heterocyclyl” (or heterocycloalkyl) means a non-aromatic saturated monocyclic or multicyclic ring system comprising 3 to 10 ring atoms, preferably 5 to 10 ring atoms, in which one or more of the atoms in the ring system is an element other than carbon, for example nitrogen (e.g. piperidyl- or pyrrolidinyl), oxygen (e.g. furanyl and tetrahydropyranyl) or sulfur (e.g. tetrahydrothiophenyl and tetrahydrothiopyranyl); and wherein the heteroatoms can be alone or in combination provided that the moiety does not contain adjacent oxygen and/or sulfur atoms present in the ring system; preferred heterocyclyl moieties contain 5 to 6 ring atoms; the prefix aza, oxa or thia before the heterocyclyl root name means that at least one nitrogen, oxygen or sulfur atom, respectively, is present as a ring atom; the heterocyclyl can be optionally substituted by one or more independently selected substituents; The nitrogen or sulfur atom of the heterocyclyl can be optionally oxidized to the corresponding N-oxide, S-oxide or S,S-dioxide (SO₂); non-limiting examples of suitable monocyclic heterocyclyl rings include piperidyl, pyrrolidinyl, piperazinyl, morpholinyl

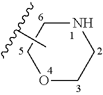

(where unless otherwise noted the moiety is bonded to the substrate through any of ring carbon atoms C2, C3, C5, or C6), thiomorpholinyl, thiazolidinyl, 1,3-dioxolanyl, 1,4-dioxanyl, tetrahydrofuranyl, tetrahydrothiophenyl, tetrahydrothiopyranyl, and the like; and polycyclicheterocyclyl compounds, for example, moieties of the structure:

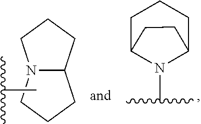

and the like.

The term “solvate” refers to a pharmaceutically acceptable solvate formed by a compound of the present disclosure with one or more solvent molecule(s). Non-limiting examples of solvent molecules include water, ethanol, acetonitrile, isopropanol, DMSO, ethyl acetate.

The term “halogen” means fluorine, chlorine, bromine, or iodine; preferred halogens, unless specified otherwise where the term is used, are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group, perhaloalkyl (or “fully halogenated” alkyl) means that all bonding positions not participating in bonding the alkyl substituent to a substrate are occupied by a halogen, for example, where the alkyl is selected to be methyl, the term perfluoroalkyl means —CF₃;

The term “hydroxyl” and “hydroxy” means an HO— group, “hydroxyalkyl” means a substituent of the formula: “HO-alkyl-” or equivalently “-alkyl-OH”, wherein the alkyl group is bonded to the substrate and may be substituted or unsubstituted as defined above; preferred hydroxyalkyl moieties comprise a lower alkyl; Non-limiting examples of suitable hydroxyalkyl groups include hydroxymethyl and 2-hydroxyethyl.

The bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

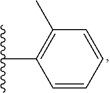 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of an atom to which it is bonded indicates that the moiety may be bonded to a substrate via any of the atoms in the moiety which are available for bonding as described in the examples above.

The line —, as a bond generally indicates a mixture of, or either of, the possible isomers, e.g., containing (R)- and (S)-stereochemical configuration.

The term “DAR” or “Drug Antibody Ratio,” as used herein, refers to the average number of linker/drug moieties attached to the antibodies present in a composition. For a composition comprising an Antibody-Drug Conjugate of the present disclosure, the DAR for the composition is the average of the “p” of all of the individual Antibody-Drug Conjugate molecules present in said composition, and this average is expressed as a decimal. As such, in some embodiments for a composition comprising an Antibody-Drug Conjugate of the present disclosure, the DAR of the composition is a decimal from 0 to 24, 0 to 8, from 0 to 7, from 0 to 6, from 0 to 5, from 0 to 4, from 0 to 3, from 0 to 2, and from 0 to 1. In additional embodiments, for a composition comprising an Antibody-Drug Conjugate of the present disclosure, the DAR of the composition is a decimal from 1 to 4, 2 to 5, 3 to 6, 4 to 7, 5 to 8, and 6 to 8. In other embodiments, for a composition comprising an Antibody-Drug Conjugate of the present disclosure, the DAR of the composition is a decimal from 1 to 3, 2 to 4, 3 to 5, 4 to 6, 5 to 7, and 6 to 8. In further embodiments, for a composition comprising an Antibody-Drug Conjugate of the present disclosure, the DAR of the composition is a decimal from 1 to 2, 2 to 3, 3 to 4, 4 to 5, 5 to 6, 6 to 7, and 7 to 8. In particular embodiments, the DAR of the composition is 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0. The term “composition” as used above, is understood to encompass pharmaceutical compositions. Average DAR can be determined by various conventional means such as UV spectroscopy, mass spectroscopy, ELISA assay, radiometric methods, hydrophobic interaction chromatography (HIC), electrophoresis and HPLC.

Furthermore, unwedged-bolded or unwedged-hashed lines are used in structures containing multiple stereocenters in order to depict relative configuration where it is known. For example:

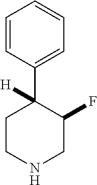 [SMILES: F[C@H]1CNCC[C@@H]1c1ccccc1]

means that the fluorine and hydrogen atoms are on the same face of the piperidine ring, but represents a mixture of, or one of, the possible isomers at right

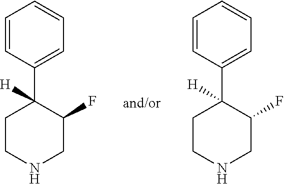 [SMILES: F[C@H]1CNCC[C@@H]1c1ccccc1 ; F[C@@H]1CNCC[C@H]1c1ccccc1]

whereas:

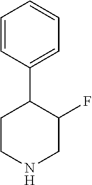 [SMILES: FC1CNCCC1c1ccccc1]

represents a mixture of, or one of, the possible isomers at right

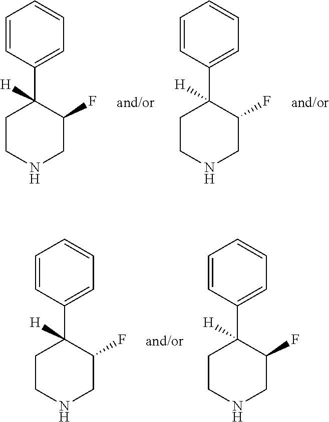 [SMILES: F[C@H]1CNCC[C@@H]1c1ccccc1 ; F[C@@H]1CNCC[C@H]1c1ccccc1 ; F[C@@H]1CNCC[C@@H]1c1ccccc1 ; F[C@H]1CNCC[C@H]1c1ccccc1]

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the present disclosure is meant to encompass all such stereoisomeric forms of these compounds.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

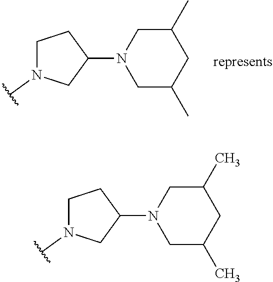

Unsatisfied valences in the text, schemes, examples, structural formulae, and any Tables herein is assumed to have a hydrogen atom or atoms of sufficient number to satisfy the valences.

One or more compounds of the disclosure may also exist as, or optionally be converted to, a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5 (1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This disclosure also includes the compounds of this disclosure in isolated and purified form obtained by routine techniques. Polymorphic forms of the compounds of Formula I, Formula II, Formula III, Formula IV, and Formula V and of the salts, solvates and prodrugs of the compounds of Formula I, Formula II, Formula III, Formula IV, and Formula V are intended to be included in the present disclosure. Certain compounds of the disclosure may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the disclosure, may exist in different tautomeric forms or in equilibrium among such forms, all such forms of the compound are embraced by, and included within the scope of the disclosure. Examples of such tautomers include, but are not limited to, ketone/enol tautomeric forms, imine-enamine tautomeric forms, and for example heteroaromatic forms such as the following moieties:

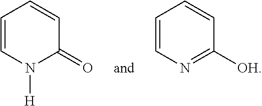 [SMILES: O=c1cccc[nH]1 ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The salts of the compounds of disclosure may be pharmaceutically acceptable salts or non-pharmaceutical salts useful in the preparation of the compounds according to the disclosure.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as formic, hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present disclosure is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the disclosure the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The term “adjacent” means connected to the same carbon atom.

The term “chemotherapeutic drug” refers to a chemical compound that can be used to treat tumors. This definition also includes antihormonal agents that act to modulate, reduce, block, or inhibit the effects of hormones that promote cancer growth, which are often in the form of systemic or holistic therapy. They can be hormones. Examples of chemotherapeutic drugs include alkylating agents, such as thiotepa; cyclosphamide (CYTOXAN™); alkyl sulfonate such as busulfan, improsulfan and piposul-fan; aziridine such as benaodopa, carboquone, meturedopa and uredopa; aziridine and methylamelamine including altretamine, triethy lenemelamine, triethy lenephosphor-amide, triethylenethiophosphoramide and trimethylolomela-mine; nitrogen mustards such as chlorambucil, chlornaphaz-ine, cholophosphamide, estramustine, ifosfamide, mechlorethamine, nitrobin hydrochloride; melphalan, novembichin, phenesterine, prednimustine, trofosfamide, uramustine; nitrosureas such as carmustine, chlorozotocin, fotemustine, lomustine, nimustine, ranimustine; antibiotic such as aclacinomycin, actinomycin, authramycin, azaser-ine, bleomycin, cactinomycin C, calicheamicin, carabicin, chromomycin, carzinophilin, chromomycin, actinomycin D, daunorubicin, detorubicin, 6-diazo-5-oxy-L-norleucine, doxorubicin, epirubicin, esorubicin, idarubicin, marcello-mycin, mitomycin, mycophenolic acid, nogalamycin, olivo-mycin, peplomycin, potfiromycin, puromycin, quelamycin, rodorubicin, streptonigrin; streptozocin, tuberculocidin, ubenimex, zinostatin, zorubicin; antimetabolites such as methotrexate, 5-fluorouracil (5-FU); folic acid analogs such as denopterin, methotrexate, pteropterin, trimetrexate; pterin analogs such as fludarabine, 6-mercaptopterin, thiomethop-terin, thioguanopterin; pyrimidine analogs such as ancit-abine, azacitidine, 6-azuridine, carmofur, cytarabine, dide-oxyuridine, doxitluridine, enocitabine, floxuridine, 5-FU; androgens such as calusterone, dromostanolong propionate, epitiostanol, mepitiostane, testolactone; anti-adrenalines such as aminoglutethimide, mitotane, trilostane; folic acid supplements such as frolinic acid; aceglatone; aldophosph-amideglycoside; aminolevulinic acid; amsacrine; bestrabu-cil; biasntrene; edatraxate; defofamine; demecolcine; diazi-quone; elfomithine; elliptinium acetate; etoglucid; gallium nitrate; hydroxyurea; lentinan; lonidamine; mitoguazone; mitoxantrone; mopidamol; nitracrine; pintostatin; phe-namet; pirarubicin; podophyllinic acid; 2-ethylhydrazide; procarbazine; PSK®; razoxane; sizofiran; spirogermanium; tenuazonic acid; triaziquone; 2,2′,2″-trichlorrotriethylamine; urethan; vindesine; dacarbazine; mannomustine; mitobroni-tol; dibromodulcitol; pipobroman; gacytosine; arabinoside (“Ara-C”); cyclophosphamide; thiotepa; taxanes such as paclitaxel (TAXOL®, Bristol-Myers Squibb Oncology, Princeton, N.J.) and docetaxel (TAXOTERE®, Rhone-Pou-lenc Rorer, Antony, France); chlorambucil; gemcitabine; 6-thioguanine; mercaptopurine; methotrexate; platinum ana-logs such as cisplatin and carboplatin; vinblastine; platinum; etoposide (VP-16); ifosfamide; mitomycin C; mitoxantrone; vincristine; vinorelbine; navelbine; novantrone; teniposide; daunorubicin; aminopterin; xeloda; ibandronate; CPT-11; topoisomerase inhibitor RFS2000; difluoromethylomithine (DMFO); retinoic acid esperamicins; capecitabine; and pharmaceutically acceptable salt, acid or derivative of any of the above substances. This definition also includes anti-hormonal agents that can modulate or inhibit the effects of hormones on tumors, such as anti-estrogens, including tamoxifen, raloxifene, aromatase inhibitor 4(5)-imidazole, 4-hydroxytamoxifen, trioxifene, keoxifene, LYII 7018, ona-pristone and Fareston; and anti-androgens such as flutamide, nilutamide, bicalutamide, leuprolide and goserelin; and pharmaceutically acceptable salt, acid or derivative of any of the above substances.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof.

As would be evident to those skilled in the art, subjects treated by the methods described herein are generally mammals, including humans and non-human animals (e.g., laboratory animals and companion animals). The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the disclosure or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the disclosure or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present disclosure are each directed to a method for the treatment a disease, disorder, or condition, or one or more symptoms thereof (“indications”) which method comprises administering to a subject in need of such treatment a therapeutically effective amount of a compound of the disclosure, or a pharmaceutically acceptable salt thereof, or a pharmaceutical composition comprising said compound or salt thereof.

In another embodiment, the present disclosure is directed to a method for the manufacture of a medicament for use in a subject comprising combining a compound of the present disclosure, or a pharmaceutically acceptable salt thereof, with a pharmaceutical carrier or diluent.

One such embodiment provides a method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma) in a subject in need thereof, said method comprising administering to a subject in need of such treatment a therapeutically effective amount of a compound of the disclosure, or a pharmaceutically acceptable salt or solvate thereof, or a pharmaceutical composition comprising said compound, salt or solvate thereof. In one such embodiment, the subject is a human.

Another aspect of the disclosure relates to a method for treating and/or preventing a tumor, comprising administering to a patient in need thereof a therapeutically effective amount of the compound, or a pharmaceutically acceptable salt or solvate thereof, or the pharmaceutical composition comprising the compound according to the present disclosure.

Combinations with additional therapeutic agents are also contemplated in the instant methods. For example, combinations of the compounds of Formula IV of the present disclosure with PPAR-γ (i.e., PPAR-gamma) agonists and PPAR-6 (i.e., PPAR-delta) agonists are useful in the treatment of certain malignancies. PPAR-γ and PPAR-6 are the nuclear peroxisome proliferator-activated receptors y and 6. PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697), or a pharmaceutically acceptable salt thereof.

Another embodiment of the instant disclosure is the use of the compounds of Formula IV of the present disclosure in combination with gene therapy for the treatment of cancer. For an overview of genetic strategies to treating cancer see Hall et al., (Am. J. Hum. Genet. 61:785-789, 1997) and Kufe et al., (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The compounds of Formula IV may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar), or a pharmaceutically acceptable salt thereof.

The compounds of Formula IV of the present disclosure may also be administered with an immunologic-enhancing drug, such as levamisole, isoprinosine and Zadaxin, or a pharmaceutically acceptable salt thereof.

The compounds of Formula IV of the present disclosure may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

The compounds of Formula IV of the present disclosure may also be useful for treating or preventing cancer in combination with Pgp and/or BCRP inhibitors including: cyclosporin A, PSC833, GF120918, cremophorEL, fumitremorgin C, Kol32, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol, or a pharmaceutically acceptable salt thereof.

The compounds of Formula IV of the present disclosure may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The compounds of Formula IV of the present disclosure may also be useful for treating or preventing breast cancer in combination with aromatase inhibitors. Examples of aromatase inhibitors include but are not limited to: anastrozole, letrozole and exemestane, or a pharmaceutically acceptable salt thereof.

The compounds of Formula IV of the present disclosure may also be useful for treating or preventing cancer in combination with siRNA therapeutics.

The compounds of Formula IV of the present disclosure may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®), abarelix (Plenaxis depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone injection®); Elliott's B Solution (Elliott's B Solution®); epirubicin (Ellence®); Epoetin alfa (epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin implant®); hydroxyurea (Hydrea); tIbritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); Olaparib (Lynparza®) vorinostat (Zolinza®) and zoledronate (Zometa®), or a pharmaceutically acceptable salt thereof.

Thus, the scope of the instant disclosure encompasses the use of the compounds of Formula IV of the present disclosure in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the disclosure is a method of treating cancer that comprises administering a therapeutically effective amount of a compound of Formula IV of the present disclosure in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a compound of Formula IV of the present disclosure is combined with one or more other anti-cancer agents for use in the prevention, treatment, control amelioration, or reduction of risk of a particular disease or condition for which the compounds of Formula IV of the present disclosure are useful. Such other active agents may be administered, by a route and in an amount commonly used therefor, prior to, contemporaneously, or sequentially with a compound of the present disclosure.

The instant disclosure also includes a pharmaceutical composition useful for treating or preventing cancer that comprises a therapeutically effective amount of compounds of Formula IV of the present disclosure and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The disclosure further relates to a method of treating cancer in a human patient comprising administration of an and a PD-1 antagonist to the patient. The compound of the disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the compounds of Formula IV of the present disclosure are administered in combination with an anti-human PD-1 antibody (or antigen-binding fragment thereof), the anti-human PD-1 antibody (or antigen-binding fragment thereof) may be administered either simultaneously with, or before or after, the compounds of Formula IV of the present disclosure. Either of the anti-human PD-1 antibody (or antigen-binding fragment thereof), and/or a compound of Formula IV of the present disclosure, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to a compound of Formula IV of the present disclosure, may be varied and will depend upon the therapeutically effective dose of each agent. Generally, a therapeutically effective dose of each will be used. Combinations including at least one anti-human PD-1 antibody (or antigen-binding fragment thereof), a compound of Formula IV of the present disclosure, and optionally other active agents will generally include a therapeutically effective dose of each active agent. In such combinations, the anti-human PD-1 antibody (or antigen-binding fragment thereof), the compounds of Formula IV, and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or a compound of Formula IV, and at least one other active agent as a combined preparation for simultaneous, separate or sequential use in treating cancer.

The disclosure also provides the use of a compound of Formula IV of the present disclosure, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with an antibody-linker-payload compound (ADC) a compound of Formula IV of the present disclosure.

The present disclosure further relates to methods of treating cancer, said method comprising administering to a subject in need thereof a combination therapy that comprises (a) a compound of Formula IV of the present disclosure, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a compound of Formula IV of the present disclosure, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically resectable.

In embodiments of the methods disclosed herein, the anti-human PD-1 antibody (or antigen binding fragment thereof) is administered by intravenous infusion or subcutaneous injection.

In one embodiment, the present disclosure provides compositions comprising a compound of Formula IV, a pharmaceutically acceptable carrier, and an anti-human PD-1 antibody (or antigen-binding fragment thereof).

In another embodiment, the present disclosure provides compositions comprising a compound of Formula IV, a pharmaceutically acceptable carrier, and pembrolizumab.

In one embodiment, the present disclosure provides compositions comprising a compound of Formula IV, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present disclosure may be employed in conjunction with anti-emetic agents to treat nausea or emesis, including acute, delayed, late-phase, and anticipatory emesis, which may result from the use of a compound of the present disclosure, alone or with radiation therapy. For the prevention or treatment of emesis, a compound of the present disclosure may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide, aprepitant, fosaprepitant, or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the compounds of Formula IV.

The compounds of Formula IV may also be administered with an agent useful in the treatment of anemia. Such an anemia treatment agent is, for example, a continuous erythropoiesis receptor activator (such as epoetin alfa).

The compounds of Formula IV may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The compounds of Formula IV may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, γ-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a compound of Formula IV in combination with radiation therapy and/or in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, provided is a kit comprising a therapeutically effective amount of a compound of Formula IV of the present disclosure or a pharmaceutically acceptable salt, solvate or ester of said compound and a pharmaceutically acceptable carrier, vehicle or diluent.

In another aspect provided is a kit comprising an amount of a compound of Formula IV of the present disclosure, and an amount of at least one additional therapeutic agent listed above, wherein the amounts of the two or more active ingredients result in a desired therapeutic effect. In one embodiment, the compound of Formula IV of the present disclosure, and the one or more additional therapeutic agents are provided in the same container. In one embodiment, the compound of Formula IV of the present disclosure, and the one or more additional therapeutic agents are provided in separate containers.

The present disclosure includes within its scope prodrugs of the compounds of this disclosure. In general, such prodrugs will be functional derivatives of the compounds of this disclosure which are readily convertible in vivo into the required compound. Thus, in the methods of treatment of the present disclosure, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this disclosure into the biological milieu.

The compounds of Formula IV may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the disclosure are effective for use in humans.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present disclosure are employed. Similarly, transdermal patches may also be used for topical administration.

The pharmaceutical composition and method of the present disclosure may further comprise other therapeutically active compounds as noted herein which are usually applied in the treatment of the above-mentioned pathological conditions.

In the treatment, prevention, control, amelioration, or reduction of risk of the conditions disclosed herein an appropriate dosage level of the compounds of this disclosure will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this disclosure are illustrated in the following Schemes and Examples. Starting materials are made according to procedures known in the art or as illustrated herein.

### Preparative Examples

The compounds of the present disclosure can be prepared according to the following schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this disclosure can be readily understood by one skilled in the art from viewing the following schemes and descriptions. Abbreviations used in the experimentals may include, but are not limited to the following:

| | |
|---|---|
| aq | Aqueous |
| AcOH | Acetic acid |
| Cbz | Carboxybenzyl |
| COMU | 1-Cyano-2-ethoxy-2-oxoethylidenaminooxy)dimethylamino- |
| | morpholino-carbenium hexafluorophosphate |
| DAR | Drug-to-antibody ratio |
| DCC | N,N′-Dicyclohexylcarbodiimide |
| DEA | Diethanolamine |
| DCM | Dichloromethane |
| DIPEA | N,N-Diisopropylethylamine |
| DMF | Dimethylformamide |
| eq. | Equivalent(s) |
| DMSO | Dimethyl sulfoxide |
| EtOAc | Ethyl acetate |
| EtOH | Ethyl alcohol |
| Et₃N/TEA | Triethylamine |
| Et₂O | Diethyl ether |
| EDTA | Ethylenediaminetetraacetic acid |
| Et₂NH | Diethylamine |
| EEDQ | N-Ethoxycarbonyl-2-ethoxy-1,2-dihydroquinoline |
| FA | Formic acid |
| Fmoc | Fluorenylmethyloxycarbonyl |
| HATU | (1-[Bis(dimethylamino)methylene]-1H-1,2,3-triazolo[4,5- |
| | b]pyridinium 3-oxide hexafluorophosphate |
| HOAt | 1-Hydroxy-7-azabenzotriazole |
| hr, h | Hour(s) |
| ¹H-NMR | Proton nuclear magnetic resonance |
| HCl | Hydrochloric acid |
| HPLC | High performance liquid chromatography |
| IPA | Isopropyl alcohol |
| K₃PO₄ | Potassium phosphate |
| LCMS | Liquid chromatography-mass spectrometry |
| mAb | Monoclonal antibody |
| MgSO₄ | Magnesium Sulfate |
| min | Minute(s) |
| MeCN | Acetonitrile |
| MeOH | Methanol |
| MS | Mass spectrometry |
| MTBE | Methyl tert-butyl ether |
| m/z | Mass to charge ratio |
| NaCl | Sodium chloride |
| Na₂SO₄ | Sodium sulfate |
| NaHCO₃ | Sodium bicarbonate |
| NaBH₄ | Sodium borohydride |
| NHS | N-hydroxysuccinimide |
| NMM | N-Methylmorpholine |
| OAc | Acetyloxy |
| Pb(OAc)₄ | Lead(IV) acetate |
| PBS | Phosphate-buffered saline |
| Pd | Palladium |
| Pd/C | Palladium on carbon |
| PyBOP | benzotriazol-1-yloxytripyrrolidinophosphonium |
| | hexafluorophosphate |
| RT | Room temperature |
| SFC | Supercritical Fluid Chromatography |
| TBSCl | tert-Butyl dimethylsilyl chloride |
| TFA | Trifluoroacetic acid |
| THF | Tetrahydrofuran |
| TLC | Thin Layer Chromatography |
| t_{R} | Retention time |

### General Experimental Information:

Unless otherwise noted, all reactions were magnetically stirred. All reagents and solvents were purchased from commercial sources and used as is unless otherwise noted. Reaction progress and synthetic intermediate analysis were assessed by LCMS (UV detection with ESI, APCI, or other mass detection) when applicable using a MeCN/water gradient with either TFA, formic acid, or NH₄HCO₃ modifier. Silica gel and reverse-phase flash column chromatography were conducted with commercially available pre-packed columns. Reverse-phase preparative HPLC purification was performed on preparative HPLC instruments with UV and MS detection using a MeCN/water gradient with either TFA, formic acid, or NH₄OH modifier. ¹H NMR spectra were collected at room temperature, and chemical shifts are reported in ppm relative to the residual proteo-solvent signals, and multiplicities, coupling constants (where applicable), and signal integrations are listed parenthetically. Unless otherwise noted, all EC₅₀ data presented in tables refers to the cytotoxicity assays that are described in the Biological Assay section.

### Synthetic Schemes, Intermediates, and Examples

The compounds of the disclosure may be prepared by methods known in the art of organic synthesis as set forth in part by the following general synthetic schemes and specific preparative examples. Starting materials are available commercially or may be prepared by known methods.

### Preparation of Intermediate I-1

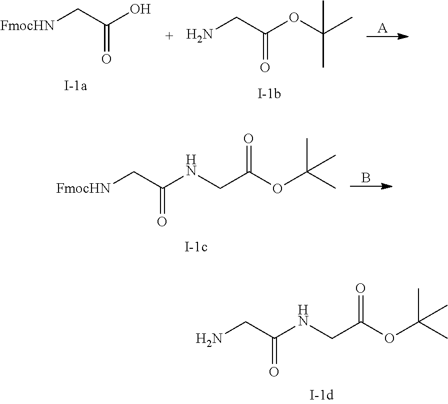

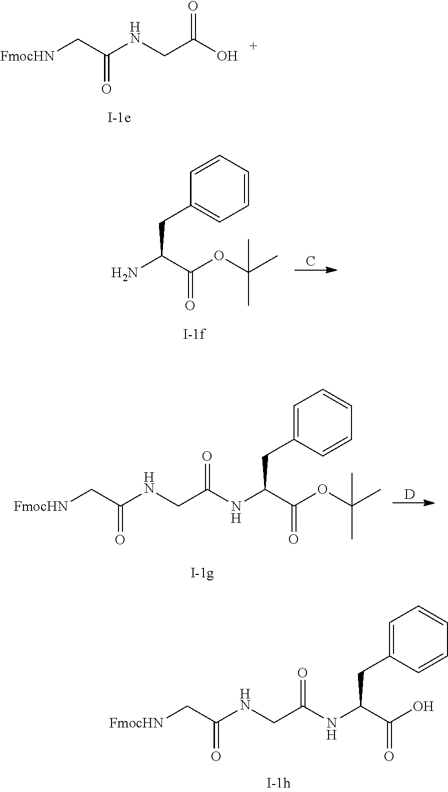

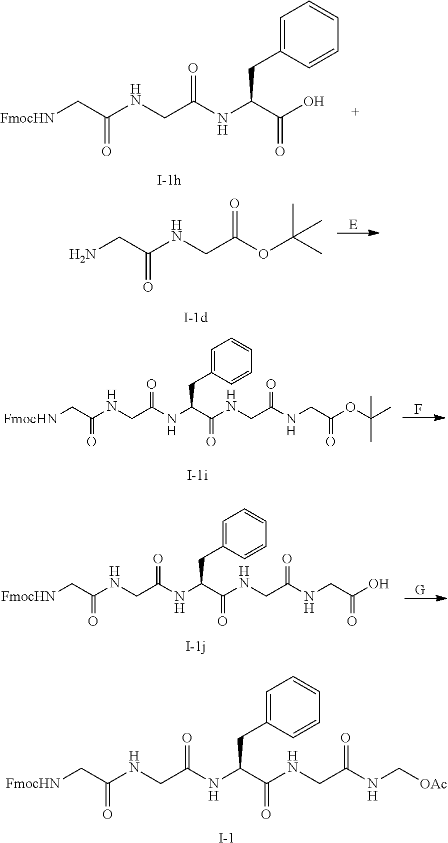

### Step A—Synthesis of Compound I-1c

To a solution of (((9H-fluoren-9-yl)methoxy)carbonyl)glycine (I-1a, 50 g, 168 mmol) in DMF (500 mL) was added HATU (70 g, 185 mmol), and the reaction was stirred at 15° C. for 5 min before DIPEA (24 g, 185 mmol) and tert-butyl glycinate (I-1b, 24 g, 185 mmol) were added. The mixture was stirred at RT for 1 h. The mixture was then diluted with water (300 mL) and extracted with EtOAc (3×200 mL). The organic layers were washed with brine (3×200 mL), and dried over MgSO₄. The volatiles were removed under reduced pressure, and the crude material was triturated with EtOAc (200 mL) to give tert-butyl (((9H-fluoren-9-yl)methoxy)carbonyl)glycylglycinate (I-1c) as a solid, which was used directly in the next reaction.

### Step B—Synthesis of Compound I-1d

To tert-butyl (((9H-fluoren-9-yl)methoxy)carbonyl)glycylglycinate (I-1c, 60 g, 146 mmol) in DMF (250 mL) was added diethylamine (78 g, 1.1 mol) at 20° C., and the mixture was stirred at 20° C. for 1 h. The extra Et₂NH was evaporated under reduced pressure, and the crude tert-butyl glycylglycinate (I-1d) DMF solution was used directly as is.

### Step C—Synthesis of Compound I-1g

To a solution of (((9H-fluoren-9-yl)methoxy)carbonyl)glycylglycine (I-1e, 65 g, 183 mmol) in DMF (650 mL) was added HATU (77 g, 202 mmol), and the reaction was stirred at 20° C. for 5 min before DIPEA (50 g, 385 mmol) and tert-butyl L-phenylalaninate (I-1f, 52 g, 202 mmol) were added. The mixture was stirred at 20° C. for 2 h. The mixture was diluted with EtOAc (500 mL) and water (200 mL), and was stirred until the product precipitated out. The solids were collected by filtration. The filter cake was slurried with MTBE (500 mL) and filtered to give tert-butyl (((9H-fluoren-9-yl)methoxy)carbonyl)glycylglycyl-L-phenylalaninate (I-1g) as a solid, which was used directly in the next reaction.

### Step D—Synthesis of Compound I-1h

To a solution of tert-butyl (((9H-fluoren-9-yl)methoxy)carbonyl)glycylglycyl-L-phenylalaninate (I-1g, 95 g, 170 mmol) in DCM (200 mL) was added TFA (200 mL), and the mixture was stirred at 25° C. for 16 h. The mixture was concentrated under reduced pressure, and the crude product was triturated with MTBE (1 L) at RT for 1 h. The solids were collected by filtration, washed with MTBE, and concentrated under reduced pressure to give (((9H-fluoren-9-yl)methoxy)carbonyl)glycylglycyl-L-phenylalanine (I-1h) as a solid, which was used directly in the next reaction.

### Step E—Synthesis of Compound I-1i

To a solution of (((9H-fluoren-9-yl)methoxy)carbonyl)glycylglycyl-L-phenylalanine (I-1h, 76 g, 152 mmol) in DMF (350 mL) was added PyBOP (83 g, 159 mmol), and the mixture was stirred at 20° C. for 30 min. Then DIPEA (21 g, 159 mmol) was added, followed by a solution of tert-butyl glycylglycinate (I-1d, 27 g, 144 mmol, 250 mL in DMF), controlling the temperature within 10-20° C. with an ice bath during addition. The reaction mixture was stirred at 10-20° C. for 2 h. The mixture was quenched with ice water (1 L) and EtOAc (500 mL), and the product precipitated out of solution. The solids were collected by filtration. The filter cake was slurried with EtOAc (1 L) at RT for 30 min. The solid was filtered and dried in vacuo to afford tert-butyl (((9H-fluoren-9-yl)methoxy)carbonyl)glycylglycyl-L-phenylalanylglycylglycinate (I-1i) as a solid, which was used directly in the next reaction.

### Step F—Synthesis of Compound I-1j

To a solution of tert-butyl (((9H-fluoren-9-yl)methoxy)carbonyl)glycylglycyl-L-phenylalanylglycylglycinate (I-1i, 65 g, 97 mmol) in DCM (120 mL) was added TFA (120 mL). The mixture was stirred at 45° C. for 2 h. The mixture was concentrated under reduced pressure. The crude product was triturated with MTBE (1 L) at RT for 20 min. The solid was collected by filtration and concentrated under reduced pressure to give (((9H-fluoren-9-yl)methoxy)carbonyl)glycylglycyl-L-phenylalanylglycylglycine (I-1j) as a solid, which was used directly in the next reaction.

### Step G—Synthesis of Compound I-1

A mixture of (((9H-fluoren-9-yl)methoxy)carbonyl)glycylglycyl-L-phenylalanylglycylglycine (I-1j, 56 g, 91 mmol) in THF (560 mL) and AcOH (56 mL) was heated to 40° C. and stirred for 30 min before Pb(OAc)₄ (170 g, 364 mmol, 95% purity) was added to the mixture. The mixture was stirred at 60° C. for 2 h. The reaction mixture was cooled to 20° C., then filtered. The filter cake was diluted with MTBE (500 mL) and water (500 mL). The two layers were then separated. The aqueous phase was extracted with EtOAc (500 mL). Then, THF (500 mL) was added to the combined organic extracts. The organics were washed with brine (2×800 mL), dried over anhydrous MgSO₄, filtered through Celite®, and concentrated under reduced pressure. The residue was purified by preparative HPLC (40-57% MeCN/water). The desired fractions were concentrated under vacuum at 30° C. The mixture was extracted with EtOAc, dried over anhydrous MgSO₄, filtered, and concentrated at 30° C. under reduced pressure to give (S)-11-benzyl-1-(9H-fluoren-9-yl)-3,6,9,12,15-pentaoxo-2-oxa-4,7,10,13,16-pentaazaheptadecan-17-yl acetate (I-1) as a solid. ¹H NMR (400 MHz, DMSO-d₆) δ 8.84 (t, J=6.9 Hz, 1H), 8.33 (br t, J=5.9 Hz, 1H), 8.15 (br d, J=8.3 Hz, 1H), 8.01 (br t, J=5.5 Hz, 1H), 7.90 (d, J=7.5 Hz, 2H), 7.72 (d, J=7.5 Hz, 2H), 7.59 (br t, J=6.0 Hz, 1H), 7.47-7.38 (m, 2H), 7.36-7.30 (m, 2H), 7.30-7.09 (m, 5H), 5.11 (dd, J=7.0, 1.3 Hz, 2H), 4.53 (td, J=8.8, 4.4 Hz, 1H), 4.36-4.12 (m, 3H), 3.90-3.50 (m, 6H), 3.06 (dd, J=13.8, 4.3 Hz, 1H), 2.79 (dd, J=13.8, 9.8 Hz, 1H), 2.00 (s, 3H).

### Preparation of Intermediate I-2

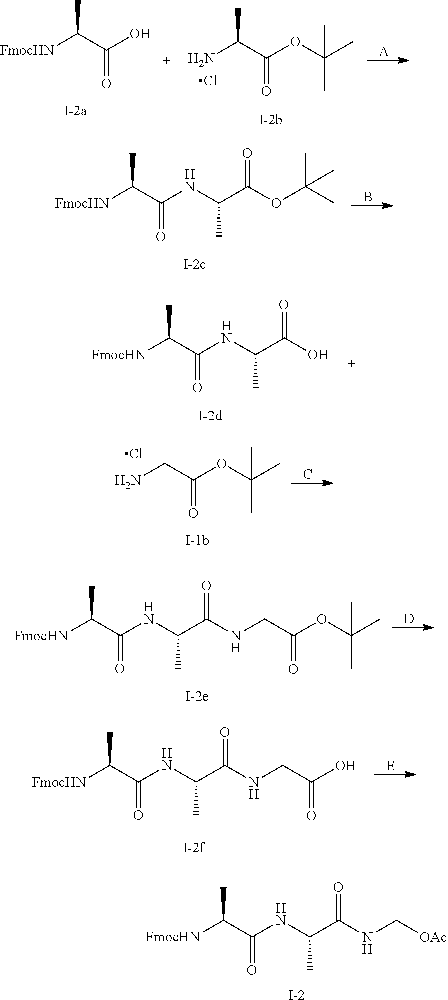

### Step A—Synthesis of Compound I-2c

To a stirred solution of (((9H-fluoren-9-yl)methoxy)carbonyl)-L-alanine (I-2a, 1.46 kg, 4.69 mol), DIPEA (1.82 kg, 1.41 mol), and tert-butyl L-alaninate hydrochloride (I-2b, 852 g, 4.69 mol) in THF (15 L) was added PyBOP (2.68 kg, 5.16 mol) in portions at 10° C. under nitrogen atmosphere. The resulting mixture was stirred for 1 h at 20° C. under nitrogen. The mixture was then concentrated under reduced pressure. The resulting mixture was diluted with water (30 L). The resulting mixture was filtered, and the filter cake was washed with water (2×5 L). The collected solid was dried in an oven under reduced pressure to afford crude tert-butyl (((9H-fluoren-9-yl)methoxy)carbonyl)-L-alanyl-L-alaninate (I-2c) as a solid, which was used directly in the next step.

### Step B—Synthesis of Compound I-2d

To a stirred solution of tert-butyl (((9H-fluoren-9-yl)methoxy)carbonyl)-L-alanyl-L-alaninate (I-2c, 2.06 kg, 4.70 mol) in DCM (6.0 L) was added trifluoroacetic acid (6.0 L) in portions at 15° C. under nitrogen atmosphere. The resulting mixture was stirred for 3 h at 20° C. under nitrogen. The resulting mixture was concentrated under reduced pressure and diluted with water (20 L). The resulting mixture was filtered, and the filter cake was washed with water (2×5 L). The collected solid was dried in an oven under reduced pressure to afford crude (((9H-fluoren-9-yl)methoxy)carbonyl)-L-alanyl-L-alanine (I-2d) as a solid, which was used directly in the next step.

### Step C—Synthesis of Compound I-2e

To a stirred solution of crude (((9H-fluoren-9-yl)methoxy)carbonyl)-L-alanyl-L-alanine (I-2d, 1.59 kg, 3.88 mol), DIPEA (1.51 kg, 11.6 mol) and tert-butyl glycinate hydrochloride (I-1b HCl, 509 g, 3.88 mol) in THF (16 L) was added PyBOP (2.22 kg, 4.27 mol) in portions at 10° C. under nitrogen atmosphere. The resulting mixture was stirred for 0.5 h at 20° C. under nitrogen. The resulting mixture was concentrated under reduced pressure, diluted with water (20 L), and filtered. The filter cake was washed with water (2×2 L). The collected solid was dried in an oven under reduced pressure to afford crude tert-butyl (((9H-fluoren-9-yl)methoxy)carbonyl)-L-alanyl-L-alanylglycinate (I-2e) as a solid, which was used directly in the next step.

### Step D—Synthesis of Compound I-2f

To a stirred solution of tert-butyl (((9H-fluoren-9-yl)methoxy)carbonyl)-L-alanyl-L-alanylglycinate (I-2e, 1.80 kg, 1.21 mol) in DCM (3.6 L) was added TFA (3.6 L) in portions at 15° C. under nitrogen atmosphere. The resulting mixture was stirred for 0.5 h at 20° C. under nitrogen. The resulting mixture was concentrated under reduced pressure, diluted with MTBE (20 L), and filtered. The filter cake was washed with MTBE (2×5 L) to afford (((9H-fluoren-9-yl)methoxy)carbonyl)-L-alanyl-L-alanylglycine (I-2f) as a solid, which was used directly in the next step.

### Step E—Synthesis of Compound I-2

A solution of (((9H-fluoren-9-yl)methoxy)carbonyl)-L-alanyl-L-alanylglycine (I-2f, 430 g, 978 mmol) in DMF (4.3 L) was treated with Cu(OAc)₂ (67 g, 367 mmol) at RT under nitrogen atmosphere, followed by the addition of acetic acid (13 g, 215 mmol) dropwise at RT. Then, lead tetraacetate (651 g, 1.47 mol) was added in portions at RT. The reaction was stirred for 3 h at 60° C. The mixture was cooled and purified by silica gel flash column chromatography (5:1 DCM:MeOH) to afford (5S,8S)-1-(9H-fluoren-9-yl)-5,8-dimethyl-3,6,9-trioxo-2-oxa-4,7,10-triazaundecan-11-yl acetate (I-2) as a solid. MS: m/z=476 [M+Na]. ¹H NMR (300 MHz, DMSO-d₆) δ 8.89 (t, J=7.1 Hz, 1H), 8.06 (d, J=7.4 Hz, 1H), 7.91 (d, J=7.5 Hz, 2H), 7.75 (t, J=6.3 Hz, 2H), 7.56 (d, J=7.6 Hz, 1H), 7.44 (td, J=7.5, 1.3 Hz, 2H), 7.36 (td, J=7.4, 1.3 Hz, 2H), 5.13 (dd, J=7.0, 2.0 Hz, 2H), 4.32-4.19 (m, 4H), 4.11 (q, J=7.2 Hz, 1H), 1.99 (s, 3H), 1.22 (d, J=2.1 Hz, 2H), 1.20 (d, J=2.0 Hz, 3H).

### Preparation of Intermediate I-3

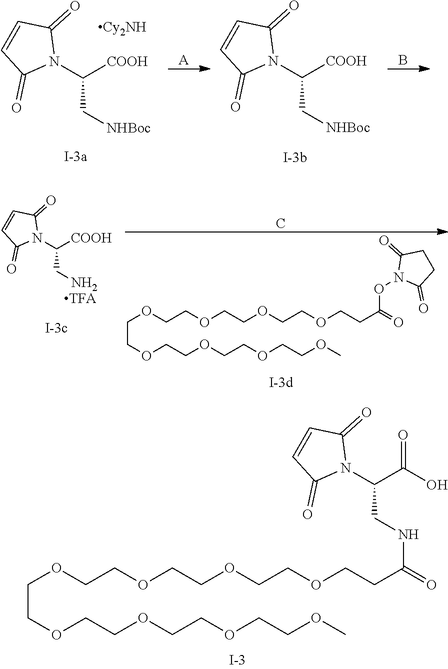

### Step A—Synthesis of Compound I-3b

A mixture of dicyclohexylamine (S)-3-((tert-butoxycarbonyl)amino)-2-(2,5-dioxo-2,5-dihydro-1H-pyrrol-1-yl)propanoate (I-3a, 1.50 g, 3.22 mmol) and formic acid (124 μl, 3.22 mmol) in DCM was directly subjected to silica gel flash column chromatography (0-40% MeOH/DCM) to afford (S)-3-((tert-butoxycarbonyl)amino)-2-(2,5-dioxo-2,5-dihydro-1H-pyrrol-1-yl)propanoic acid (I-3b). ¹H NMR (500 MHz, DMSO-d₆) δ 13.27 (s, 1H), 7.09 (s, 2H), 6.97 (t, J=6.2 Hz, 1H), 4.59 (dd, J=10.5, 3.8 Hz, 1H), 3.59-3.52 (m, 1H), 3.45-3.38 (m, 1H), 1.32 (s, 9H).

### Step B—Synthesis of Compound I-3c

(S)-3-((tert-butoxycarbonyl)amino)-2-(2,5-dioxo-2,5-dihydro-1H-pyrrol-1-yl)propanoic acid (I-3b, 350 mg, 1.2 mmol) was dissolved in DCM (820 μl) and TFA (410 μl, 5.3 mmol). The reaction was stirred at RT for 2 h, and concentrated under reduced pressure to an oil. The material was then precipitated with Et₂O (20 mL), followed by sonication. The precipitate was collected via vacuum filtration, then redissolved in MeCN and re-concentrated under reduced pressure to afford (S)-3-amino-2-(2,5-dioxo-2,5-dihydro-1H-pyrrol-1-yl)propanoic acid-2,2,2-trifluoroacetic acid (I-3c) as a solid. ¹H NMR (500 MHz, DMSO-d₆) δ 13.93 (s, 1H), 7.99 (s, 3H), 7.18 (s, 2H), 4.86 (dd, J=10.0, 4.7 Hz, 1H), 3.47 (dd, J=13.3, 4.6 Hz, 1H), 3.34 (dd, J=13.1, 10.3 Hz, 2H, overlaps with water signal).

### Step C—Synthesis of Compound I-3

A solution of (S)-3-amino-2-(2,5-dioxo-2,5-dihydro-1H-pyrrol-1-yl)propanoic acid-2,2,2-trifluoroacetic acid (I-3c, 49 mg, 0.16 mmol) and DIPEA (0.057 mL, 0.33 mmol) in DMF (0.70 mL) was added to m-dPEG®8-NHS ester (I-3d, 100 mg, 0.20 mmol). The reaction was stirred at RT for ˜10 minutes (monitored by LCMS). The material was directly subjected to reverse phase flash column chromatography (0-95% MeCN/H₂O+0.1% FA) to afford (S)-29-(2,5-dioxo-2,5-dihydro-1H-pyrrol-1-yl)-26-oxo-2,5,8,11,14,17,20,23-octaoxa-27-azatriacontan-30-oic acid (I-3) as an oil. ¹H NMR (500 MHz, DMSO-d₆) δ 13.35 (s, 1H), 7.99 (t, J=5.9 Hz, 1H), 7.09 (s, 2H), 4.62-4.56 (m, 1H), 3.81-3.75 (m, 1H), 3.55-3.49 (m, 24H), 3.48-3.46 (m, 3H), 3.44-3.41 (m, 4H), 3.24 (s, 3H), 2.24-2.17 (m, 2H).

The following compound of the present disclosure in Table 3 were made using similar methods described in Example I-3 with subtle variations in reaction times and/or solvents, and substituting appropriate reactants and/or reagents (I-3d with other commercially available NHS esters):

TABLE 3

| | | MS |
|---|---|---|
| Intermediate | Structure | [M + H] |
| I-4 | 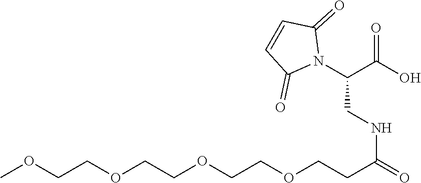 [SMILES: COCCOCCOCCOCCC(=O)NC[C@@H](C(=O)O)N1C(=O)C=CC1=O] | 403 |

### Preparation of Intermediate I-5

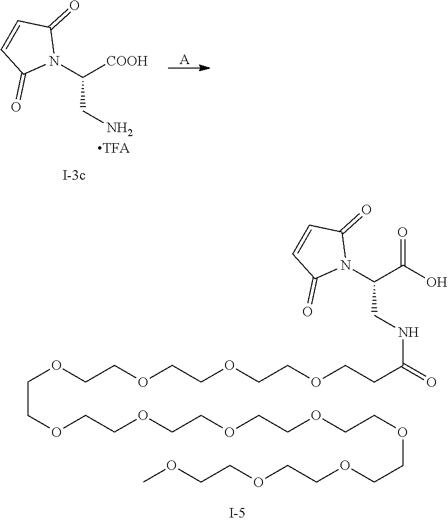

### Step a Synthesis of Compound I-5

To a solution of 2,5,8,11,14,17,20,23,26,29,32,35-dodecaoxaoctatriacontan-38-oic acid (0.095 g, 0.16 mmol) and HATU (0.061 g, 0.16 mmol) in DMF (2 mL) was added N-methylmorpholine (0.050 mL, 0.46 mmol). The reaction was stirred at RT for ˜5 min before (S)-3-amino-2-(2,5-dioxo-2,5-dihydro-1H-pyrrol-1-yl)propanoic acid, TFA (I-3c, 0.048 g, 0.16 mmol) was added. The reaction was stirred at RT for ˜2.5 h. The mixture was then concentrated under reduced pressure. The resulting (S)-41-(2,5-dioxo-2,5-dihydro-1H-pyrrol-1-yl)-38-oxo-2,5,8,11,14,17,20,23,26,29,32,35-dodecaoxa-39-azadotetracontan-42-oic acid (I-5) oil was used directly as is.

### Preparation of Intermediate I-6

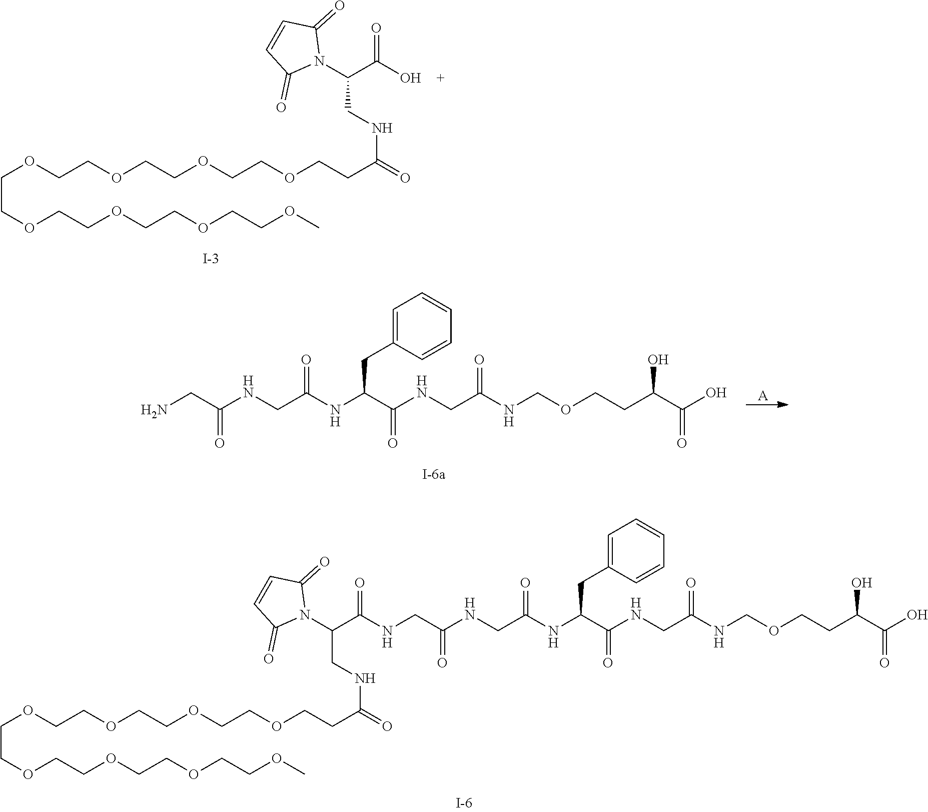

### Step a Synthesis of Compound I-6

To a solution of (S)-29-(2,5-dioxo-2,5-dihydro-1H-pyrrol-1-yl)-26-oxo-2,5,8,11,14,17,20,23-octaoxa-27-azatriacontan-30-oic acid (I-3, 0.17 g, 0.30 mmol) and 2,6-lutidine (0.094 mL, 0.81 mmol) in DMF (2.0 mL) was added COMU (0.12 g, 0.27 mmol) at 0° C. The reaction was stirred at 0° C. for ˜25 minutes before a solution of (7S,17R)-1-amino-7-benzyl-17-hydroxy-2,5,8,11-tetraoxo-14-oxa-3,6,9,12-tetraazaoctadecan-18-oic acid (I-6a, 0.13 g, 0.27 mmol) in DMF (3.0 mL) was added. The reaction was stirred at 0° C. for ˜2 h before allowing to fully warm to RT. At ˜3 h 15 min, the solution was concentrated under reduced pressure. The resulting material was washed with EtOAc 3× (in first wash some ether added). The resulting crude (29S,38S,48R)-38-benzyl-29-(2,5-dioxo-2,5-dihydro-1H-pyrrol-1-yl)-48-hydroxy-26,30,33,36,39,42-hexaoxo-2,5,8,11,14,17,20,23,45-nonaoxa-27,31,34,37,40,43-hexaazanonatetracontan-49-oic acid (I-6) was dried and used directly in the next reaction.

### Preparation of Intermediate I-7

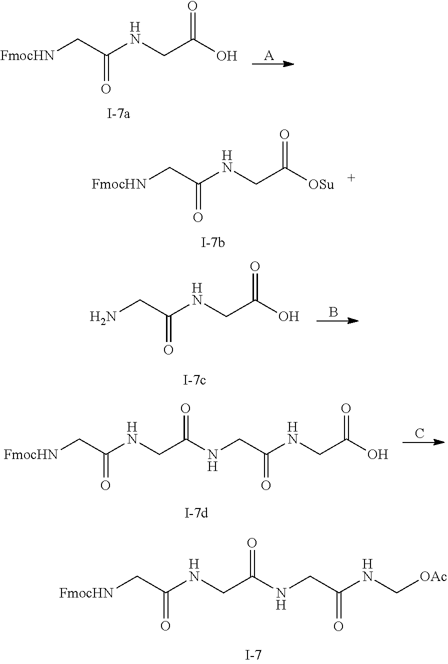

### Step A—Synthesis of Compound I-7b

To a solution of (((9H-fluoren-9-yl)methoxy)carbonyl)glycylglycine (I-7a, 18 g, 50 mmol) and 1-hydroxypyrrolidine-2,5-dione (6.9 g, 60 mmol) in 1,4-dioxane (200 mL) and stirred in an ice bath was slowly added a solution of DCC (12 g, 60 mmol) in dry 1,4-dioxane (50 mL). The mixture was stirred at 25° C. for 20 h. After cooling in the refrigerator for 1 h, the precipitate was removed to afford 2,5-dioxopyrrolidin-1-yl (((9H-fluoren-9-yl)methoxy)carbonyl)glycylglycinate (I-7b) solution in 1,4-dioxane, which was used without further purification.

### Step B—Synthesis of Compound I-7d

Glycylglycine (I-7c, 12 g, 93 mmol) and sodium bicarbonate (7.8 g, 93 mmol) were dissolved in water (150 mL). This aqueous solution was added to a solution of 2,5-dioxopyrrolidin-1-yl (((9H-fluoren-9-yl)methoxy)carbonyl)glycylglycinate (I-7b, 35 g, 78 mmol) in 1,4-dioxane (380 mL). The mixture was stirred at 25° C. for 18 h. The solution was concentrated under reduced pressure. At the same time, the pH value was adjusted to pH 2-3 by addition of 10% aqueous citric acid. The resulting white solid was precipitated, filtered, and washed with cold water. Drying the solid under vacuum afforded (((9H-fluoren-9-yl)methoxy)carbonyl)glycylglycylglycylglycine (I-7d), which was moved forward directly to the next step.

### Step C—Synthesis of Compound I-7

To solution of (((9H-fluoren-9-yl)methoxy)carbonyl)glycylglycylglycylglycine (I-7d, 5.0 g, 11 mmol) in DMF (50 mL) was added copper(II) acetate (0.78 g, 4.3 mmol), acetic acid (1.4 mL, 25 mmol) and lead(IV) tetraacetate (24 g, 53 mmol). The reaction was heated to 60° C. for 1 h. The mixture was filtered at 60° C., then the filtrate was added to water (100 mL), filtered, and the resulting cake solid was dissolved in DMF (50 mL) again, then heated to 60° C., then filtered again. The filtrate was added to ice water (150 mL), then the precipitate was filtered and the filter cake was dried under vacuum to give 1-(9H-fluoren-9-yl)-3,6,9,12-tetraoxo-2-oxa-4,7,10,13-tetraazatetradecan-14-yl acetate (I-7) as a solid. MS: m/z=505 [M+Na]. ¹H NMR (400 MHz, DMSO-d₆) δ 8.90 (br t, J=6.9 Hz, 1H), 8.16 (td, J=5.7, 11.8 Hz, 2H), 7.89 (d, J=7.5 Hz, 2H), 7.72 (d, J=7.4 Hz, 2H), 7.58 (br t, J=6.0 Hz, 1H), 7.45-7.39 (m, 2H), 7.36-7.30 (m, 2H), 5.09 (d, J=7.0 Hz, 2H), 4.33-4.27 (m, 2H), 4.25-4.21 (m, 1H), 3.78-3.72 (m, 4H), 3.70-3.65 (m, 2H), 1.98 (s, 3H).

### Preparation of Intermediate I-8

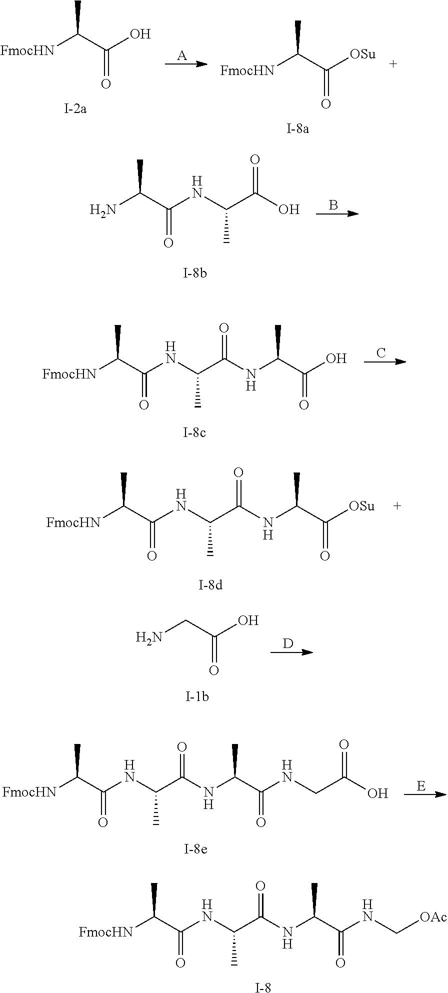

### Step A—Synthesis of Compound I-8a

A solution of (((9H-fluoren-9-yl)methoxy)carbonyl)-L-alanine (I-2a, 31 g, 100 mmol) and 1-hydroxypyrrolidine-2,5-dione (14 g, 120 mmol) in 1,4-dioxane (400 mL) was stirred at 0° C. A solution of DCC (25 g, 120 mmol) in 1,4-dioxane (100 mL) was then slowly added. The mixture was stirred at 0° C. for 4 h, and then at 20° C. for a further 18 h. After cooling in the refrigerator for 1 h, the precipitate was removed to afford 2,5-dioxopyrrolidin-1-yl (((9H-fluoren-9-yl)methoxy)carbonyl)-L-alaninate (I-8a) solution in 1,4-dioxane (500 mL), which was used directly in the next step.

### Step B—Synthesis of Compound I-8c

L-Alanyl-L-Alanine (I-8b, 19 g, 120 mmol) and sodium bicarbonate (9.87 g, 118 mmol) were dissolved in water (40 mL). The aqueous solution was added to a solution of 2,5-dioxopyrrolidin-1-yl (((9H-fluoren-9-yl)methoxy)carbonyl)-L-alaninate (I-8a, 40 g, 98 mmol) in 1,4-dioxane (160 mL). The mixture was stirred at about 25° C. for 17 h. The solution was concentrated to a small volume (10 mL), and at the same time, the pH value was adjusted close to pH 2-3 by addition of 10% aqueous citric acid. The resultant gelatinous precipitate was filtered and washed with cold water. The filter cake was dried under vacuum to give (((9H-fluoren-9-yl)methoxy)carbonyl)-L-alanyl-L-alanyl-L-alanine (I-8c) as a solid.

### Step C—Synthesis of Compound I-8d

A solution of (((9H-fluoren-9-yl)methoxy)carbonyl)-L-alanyl-L-alanyl-L-alanine (I-8c, 40 g, 88 mmol) and 1-hydroxypyrrolidine-2,5-dione (12 g, 110 mmol) in 1,4-dioxane (500 mL) was stirred at 0° C. Then, a solution of DCC (22 g, 110 mmol) in 1,4-dioxane (100 mL) was slowly added. The mixture was stirred at 0° C. for 0.5 h, and then at 25° C. for a further 18 h. After cooling in the refrigerator for 1 h, the precipitate was removed, affording a 2,5-dioxopyrrolidin-1-yl (((9H-fluoren-9-yl)methoxy)carbonyl)-L-alanyl-L-alanyl-L-alaninate (I-8d) solution in 1,4-dioxane (600 mL), which was used directly in the next step.

### Step D—Synthesis of Compound I-8e

Glycine (I-1b, 7.5 g, 100 mmol) and sodium bicarbonate (8.4 g, 100 mmol) were dissolved in water (300 mL). This aqueous solution was added to a solution of 2,5-dioxopyrrolidin-1-yl (((9H-fluoren-9-yl)methoxy)carbonyl)-L-alanyl-L-alanyl-L-alaninate (I-8d, 46 g, 84 mmol) in 1,4-dioxane (800 mL) for 0.5 h at 0° C. The mixture was stirred at 25° C. for 18 h. The solution was concentrated under reduced pressure, and at the same time, the pH value was adjusted to pH 2-3 by addition of 10% aqueous citric acid. The resultant gelatinous precipitate was filtered, and washed with cold water. Drying under vacuum afforded (((9H-fluoren-9-yl)methoxy)carbonyl)-L-alanyl-L-alanyl-L-alanylglycine (I-8e) as a solid, which was used directly in the next step.

### Step E—Synthesis of Compound I-8

To a solution of (((9H-fluoren-9-yl)methoxy)carbonyl)-L-alanyl-L-alanyl-L-alanylglycine (I-8e, 4.0 g, 7.8 mmol) in DMF (100 mL) were added copper(II) acetate (0.57 g, 3.1 mmol), acetic acid (1.0 mL, 18 mmol), and lead(IV) tetraacetate (17 g, 39 mmol). The reaction was heated to 60° C. for 1 h. The mixture was filtered at 60° C., then the filtrate was added to water (100 mL), and filtered. The cake solid was dissolved in DMF (100 mL) again, then heated to 60° C., then filtered again, and the filtrate was added to ice water (300 mL). The precipitate was filtered and the filtrate was added to DCM/MeOH (4:1, 100 mL). The resulting precipitate was centrifuged and the filter cake was concentrated under reduced pressure to give (5S,8S,11S)-1-(9H-fluoren-9-yl)-5,8,11-trimethyl-3,6,9,12-tetraoxo-2-oxa-4,7,10,13-tetraazatetradecan-14-yl acetate (I-8) as a solid. MS: m/z=547 [M+Na]. ¹H NMR (400 MHz, DMSO-d₆) δ 8.86 (br t, J=6.8 Hz, 1H), 7.98 (br dd, J=7.3, 16.1 Hz, 2H), 7.89 (d, J=7.5 Hz, 2H), 7.72 (br t, J=7.0 Hz, 2H), 7.54 (br d, J=7.5 Hz, 1H), 7.44-7.39 (m, 2H), 7.36-7.31 (m, 2H), 5.14-5.03 (m, 2H), 4.28-4.19 (m, 5H), 4.13-4.01 (m, 1H), 1.98 (s, 3H), 1.22-1.18 (m, 9H).

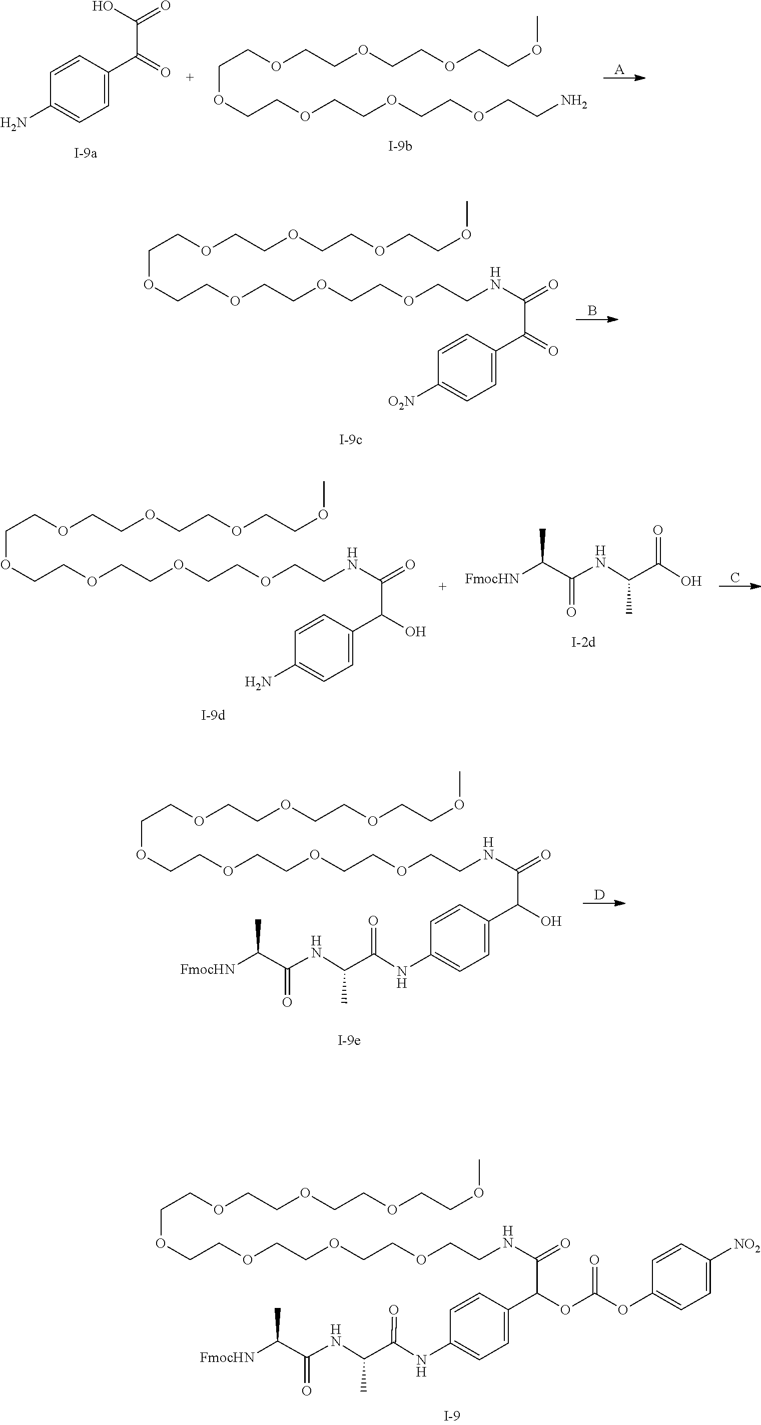

### Preparation of Intermediate I-9

### Step A—Synthesis of Compound I-9c

A mixture of 2-(4-nitrophenyl)-2-oxoacetic acid (I-9a, 0.55 g, 2.8 mmol), HATU (1.03 g, 2.7 mmol) and NMM (0.66 mL, 6.0 mmol) in DMF (6.0 mL) was stirred at RT for 2 min, at which point 2,5,8,11,14,17,20,23-octaoxapentacosan-25-amine (I-9b, 770 mg, 2.0 mmol) in DMF (1.0 mL) was added. The reaction mixture was stirred at RT for 20 min, then concentrated under reduced pressure. The residue was purified by reverse phase column chromatography (0-70% MeCN/H₂O with 0.1% formic acid modifier) to afford 2-(4-nitrophenyl)-2-oxo-N-(2,5,8,11,14,17,20,23-octaoxapentacosan-25-yl)acetamide (I-9c) as an oil. MS: m/z=561 [M+H].

### Step B—Synthesis of Compound I-9d

A mixture of 2-(4-nitrophenyl)-2-oxo-N-(2,5,8,11,14,17,20,23-octaoxapentacosan-25-yl)acetamide (I-9c, 760 mg, 1.36 mmol), iron (380 mg, 6.78 mmol) and ammonium chloride (435 mg, 8.13 mmol) in ethanol (5.8 mL) and water (1.9 mL) was stirred at 70° C. for 3 h. The reaction mixture was filtered, rinsing with MeOH, and concentrated under reduced pressure. After redissolving in MeCN, the mixture was filtered and concentrated under reduced pressure again. The residue was dissolved in MeOH (5.8 mL), cooled to 0° C. and treated with sodium borohydride (260 mg, 6.78 mmol) portionwise over 2 min. After gas evolution stopped, the mixture was concentrated under reduced pressure, suspended in water and purified by reverse phase column chromatography (0-90% MeCN/H₂O with 0.1% formic acid modifier) to afford 2-(4-aminophenyl)-2-hydroxy-N-(2,5,8,11,14,17,20,23-octaoxapentacosan-25-yl)acetamide (I-9d) as a thick oil. MS: m/z=533 [M+H].

### Step C—Synthesis of Compound I-9e

A mixture of 2-(4-aminophenyl)-2-hydroxy-N-(2,5,8,11,14,17,20,23-octaoxapentacosan-25-yl)acetamide (I-9d, 200 mg, 0.38 mmol), (((9H-fluoren-9-yl)methoxy)carbonyl)-L-alanyl-L-alanine (I-2d, 170 mg, 0.45 mmol) and EEDQ (185 mg, 0.75 mmol) in DCM (1.67 mL) and MeOH (0.83 mL) was stirred at RT for 2 h. The reaction mixture was the concentrated under reduced pressure and purified by reverse phase column chromatography (10-100% MeCN/H₂O with 0.1% formic acid modifier) to afford (9H-fluoren-9-yl)methyl ((2S)-1-(((2S)-1-((4-(28-hydroxy-27-oxo-2,5,8,11,14,17,20,23-octaoxa-26-azaoctacosan-28-yl)phenyl)amino)-1-oxopropan-2-yl)amino)-1-oxopropan-2-yl)carbamate (I-9e) as a solid. MS: m/z=879 [M+H−H₂O].

### Step D—Synthesis of Compound I-9

To a solution of (9H-fluoren-9-yl)methyl ((2S)-1-(((2S)-1-((4-(28-hydroxy-27-oxo-2,5,8,11,14,17,20,23-octaoxa-26-azaoctacosan-28-yl)phenyl)amino)-1-oxopropan-2-yl)amino)-1-oxopropan-2-yl)carbamate (I-9e, 200 mg, 0.22 mmol) in DMF (1.1 mL) was added bis(4-nitrophenyl) carbonate (135 mg, 0.45 mmol). The reaction mixture was stirred at RT for 26 h. The mixture was then directly purified by reverse phase column chromatography (10-95% MeCN/H₂O with 0.1% formic acid modifier) to afford (9H-fluoren-9-yl)methyl ((2S)-1-(((2S)-1-((4-(1-(4-nitrophenoxy)-1,4-dioxo-2,8,11,14,17,20,23,26,29-nonaoxa-5-azatriacontan-3-yl)phenyl)amino)-1-oxopropan-2-yl)amino)-1-oxopropan-2-yl)carbamate (I-9). MS: m/z=1084 [M+Na]. ¹H NMR (500 MHz, DMSO-d₆) δ 10.05 (s, 1H), 8.48 (t, J=5.5 Hz, 1H), 8.33 (d, J=9.2 Hz, 2H), 8.13 (d, J=7.0 Hz, 1H), 7.89 (d, J=7.5 Hz, 2H), 7.72 (t, J=8.4 Hz, 2H), 7.63 (d, J=8.4 Hz, 2H), 7.59-7.52 (m, 3H), 7.45 (d, J=8.7 Hz, 2H), 7.41 (t, J=7.5 Hz, 2H), 7.33 (t, J=7.5 Hz, 2H), 5.89 (s, 1H), 4.45-4.37 (m, 1H), 4.32-4.17 (m, 3H), 4.14-4.06 (m, 1H), 3.52-3.37 (m, 30H), 3.26-3.20 (m, 5H), 1.31 (d, J=7.1 Hz, 3H), 1.23 (d, J=7.1 Hz, 3H).

### Preparation of Intermediate I-10

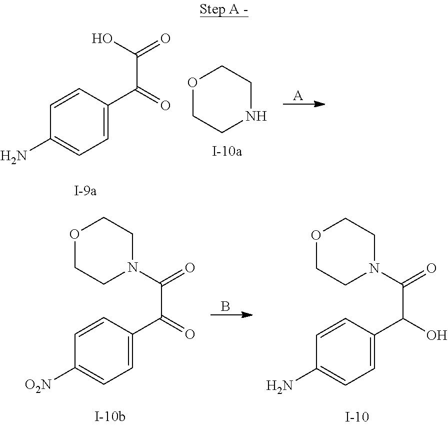

### Synthesis of Compound I-10b

A mixture of 2-(4-nitrophenyl)-2-oxoacetic acid (I-9a, 0.98 g, 5.0 mmol), 1-propane phosphonic anhydride (50% in DMF, 6.0 mL, 10 mmol) and DIPEA (2.6 mL, 15 mmol) in DCM (17 mL) was stirred at RT for 5 minutes. The stirred mixture was then cooled to 0° C., at which point morpholine (I-10a, 1.3 mL, 15 mmol) was added dropwise. The reaction mixture was stirred at RT overnight, concentrated under reduced pressure, and purified by silica gel column chromatography (0-10% MeOH/DCM) to afford 1-morpholino-2-(4-nitrophenyl)ethane-1,2-dione (I-10b) as a solid. MS: m/z=265 [M+H].

### Step B—Synthesis of Compound I-10

A mixture of 1-morpholino-2-(4-nitrophenyl)ethane-1,2-dione (I-10b, 1.25 g, 4.7 mmol) and 10 wt % Pd/C (0.25 g, 0.24 mmol Pd) in DCM (12 mL) and MeOH (12 mL) was stirred under H₂ (1.0 atm) for 2 days. After adding another portion of 10 wt % Pd/C (0.25 g, 0.24 mmol Pd), the reaction mixture was stirred under H₂ (1.0 atm) for 1 day. The mixture was filtered through Celite, rinsing with MeOH and DCM, concentrated under reduced pressure and purified by silica gel column chromatography (0-15% MeOH/DCM) to afford 2-(4-aminophenyl)-2-hydroxy-1-morpholinoethan-1-one (I-10). MS: m/z=219 [M+H−H₂O]. ¹H NMR (500 MHz, DMSO-d₆) δ 6.97 (d, J=8.3 Hz, 2H), 6.52 (d, J=8.3 Hz, 2H), 5.17 (s, 3H), 3.61-3.34 (m, 7H), 3.25-3.11 (m, 2H).

### Intermediate I-11

### Preparation of Intermediate I-11

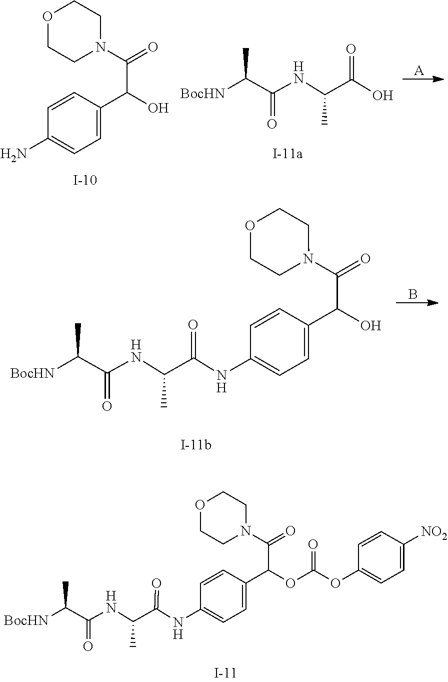

### Step A—Synthesis of Compound I-11b

A mixture of 2-(4-aminophenyl)-2-hydroxy-1-morpholinoethan-1-one (I-10, 0.68 g, 2.9 mmol), (tert-butoxycarbonyl)-L-alanyl-L-alanine (I-11a, 1.12 g, 4.3 mmol) and EEDQ (1.42 g, 5.8 mmol) in DCM (6.4 mL) and MeOH (3.2 mL) was stirred at RT overnight. The reaction mixture was the concentrated under reduced pressure and purified by silica gel column chromatography (0-15% MeOH/DCM) to afford tert-butyl ((2S)-1-(((2S)-1-((4-(1-hydroxy-2-morpholino-2-oxoethyl)phenyl)amino)-1-oxopropan-2-yl)amino)-1-oxopropan-2-yl)carbamate (I-11b) as a solid. MS: m/z=461 [M+H−H₂O].

### Step B—Synthesis of Compound I-11

A mixture of tert-butyl ((2S)-1-(((2S)-1-((4-(1-hydroxy-2-morpholino-2-oxoethyl)phenyl)amino)-1-oxopropan-2-yl)amino)-1-oxopropan-2-yl)carbamate (I-11b, 1.08 g, 2.3 mmol), bis(4-nitrophenyl) carbonate (1.37 g, 4.5 mmol) and DIPEA (0.39 mL, 2.3 mmol) in MeCN (4.5 mL) and DMF (2.0 mL) was stirred at RT for 3 h. The reaction mixture was then directly purified by reverse phase column chromatography (10-90% MeCN/H₂O with 0.1% formic acid modifier) to afford tert-butyl ((2S)-1-(((2S)-1-((4-(2-morpholino-1-(((4-nitrophenoxy)carbonyl)oxy)-2-oxoethyl)phenyl)amino)-1-oxopropan-2-yl)amino)-1-oxopropan-2-yl)carbamate (I-11) as a solid. MS: m/z=666 [M+Na]. ¹H NMR (500 MHz, DMSO-d₆) δ 10.11 (s, 1H), 8.33 (d, J=9.1 Hz, 2H), 8.04 (d, J=7.1 Hz, 1H), 7.68 (d, J=8.5 Hz, 2H), 7.55 (d, J=9.1 Hz, 2H), 7.48 (d, J=8.5 Hz, 2H), 6.97 (d, J=7.3 Hz, 1H), 6.46 (s, 1H), 4.44-4.34 (m, 1H), 4.04-3.93 (m, 1H), 3.57-3.40 (m, 6H), 3.24-3.16 (m, 1H), 3.13-3.06 (m, 1H), 1.38 (s, 9H), 1.31 (d, J=7.1 Hz, 3H), 1.18 (d, J=7.1 Hz, 3H).

The following compounds of the present disclosure in Table 4 were made using similar methods described in Example I-11 with subtle variations in reaction times and/or solvents, and substituting appropriate reactants and/or reagents:

TABLE 4

| | | MS |
|---|---|---|
| Intermediate | Structure | [M + H] |
| I-12 | 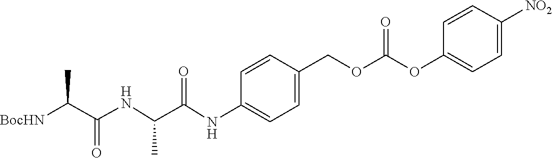 [SMILES: C[C@H](NC(=O)OC(C)(C)C)C(=O)N[C@@H](C)C(=O)Nc1ccc(COC(=O)Oc2ccc([N+](=O)[O-])cc2)cc1] | See NMR Data |
| | Step A (I-11) - DCM as solvent | |
| | ¹H NMR (500 MHz, CDCl₃) δ 8.72 (s, 1H), 8.39- | |
| | 8.20 (m, 2H), 7.68 (d, J = 7.9 Hz, 2H), 7.50-7.33 (m, | |
| | 4H), 6.67 (d, J = 7.5 Hz, 1H), 5.24 (s, 2H), 4.91 (s, | |
| | 1H), 4.61 (p, J = 7.2 Hz, 1H), 4.15 (p, J = 6.9 Hz, 1H), | |
| | 1.48 (d, J = 7.1 Hz, 3H), 1.44 (s, 9H), 1.41 (d, J = 7.2 | |
| | Hz, 3H). | |
| I-13 | 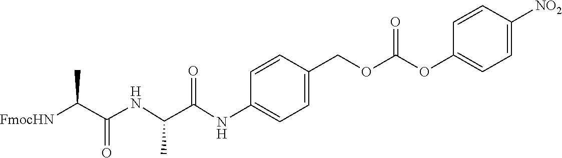 | 653 |
| | Step A (I-11) - 5:1 DCM/MeOH as solvent | |
| | Step B (I-11) - DMF as solvent | |

### Intermediate I-14

### Preparation of Intermediate I-14

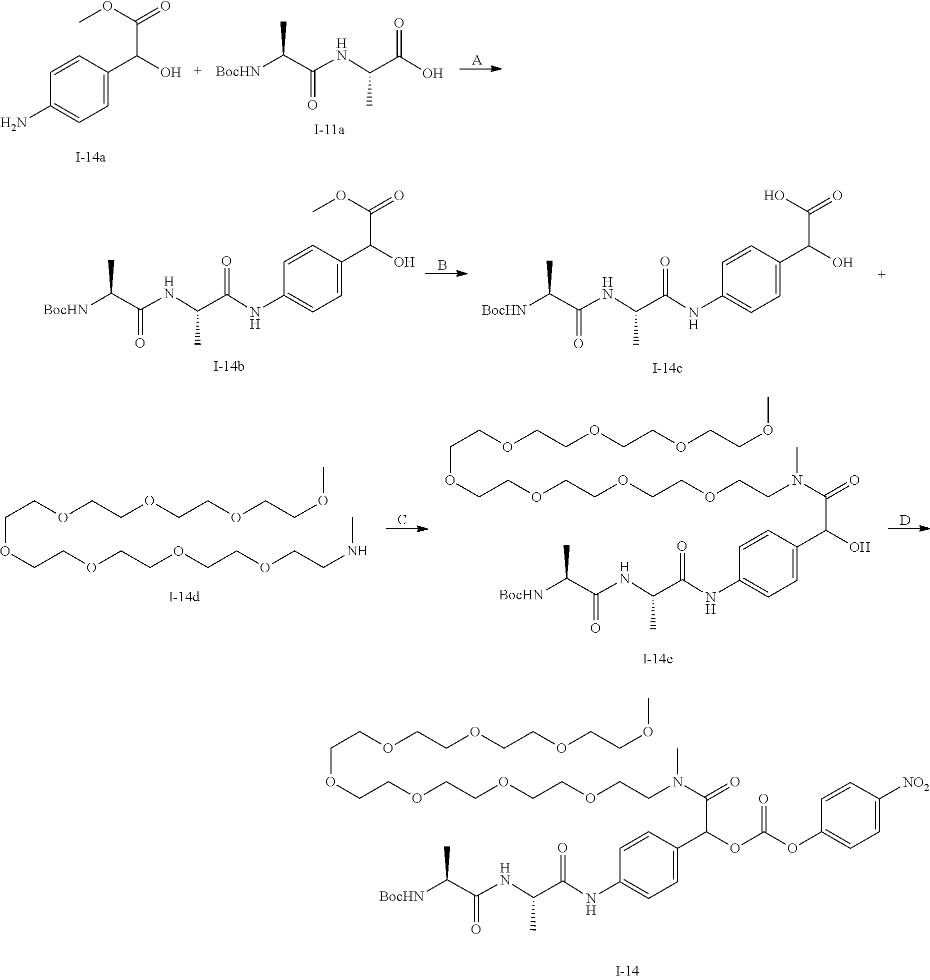

### Step A—Synthesis of Compound I-14b

A mixture of methyl 2-(4-aminophenyl)-2-hydroxyacetate (I-14a, 0.62 g, 3.4 mmol), (tert-butoxycarbonyl)-L-alanyl-L-alanine (I-11a, 1.33 g, 5.1 mmol) and EEDQ (1.68 g, 6.8 mmol) in DCM (14 mL) and MeOH (7 mL) was stirred at RT for 3 days. The reaction mixture was the concentrated under reduced pressure and purified by silica gel column chromatography (0-10% MeOH/DCM) to afford methyl 2-(4-((S)-2-((S)-2-((tert-butoxycarbonyl)amino)propanamido)propanamido)phenyl)-2-hydroxyacetate (I-14b). MS: m/z=446 [M+Na].

### Step B—Synthesis of Compound I-14c

A mixture of methyl 2-(4-((S)-2-((S)-2-((tert-butoxycarbonyl)amino)propanamido)propanamido)phenyl)-2-hydroxyacetate (I-14b, 870 mg, 2.05 mmol) and lithium hydroxide (98 mg, 4.1 mmol) in MeOH (12 mL) and water (6 mL) was stirred at RT overnight. Then mixture was then neutralized with aqueous 1M HCl (5 mL) and shaken with DCM (100 mL). The aqueous layer was back-extracted with 10% MeOH/DCM. The combined organic layers were washed with brine twice, and the second brine layer was back-extracted with IPA/DCM. The combined organic layers were dried over MgSO₄, filtered and concentrated under reduced pressure to afford 2-(4-((S)-2-((S)-2-((tert-butoxycarbonyl)amino)propanamido)propanamido)phenyl)-2-hydroxyacetic acid (I-14c) as a solid. MS: m/z=432 [M+Na].

### Step C—Synthesis of Compound I-14e

To a stirred solution of 2-(4-((S)-2-((S)-2-((tert-butoxycarbonyl)amino)propanamido)propanamido)phenyl)-2-hydroxyacetic acid (I-14c, 580 mg, 1.4 mmol), N-methyl-2,5,8,11,14,17,20,23-octaoxapentacosan-25-amine (I-14d, 730 mg, 1.8 mmol) and NMM (0.31 mL, 1.8 mmol) in DMF (2.8 mL) was added HATU (700 mg, 1.8 mmol). The mixture was stirred at RT for 20 minutes, then directly purified by reverse phase column chromatography (10-60% MeCN/H₂O with 0.1% formic acid modifier) to afford tert-butyl ((2S)-1-(((2S)-1-((4-(28-hydroxy-26-methyl-27-oxo-2,5,8,11,14,17,20,23-octaoxa-26-azaoctacosan-28-yl)phenyl)amino)-1-oxopropan-2-yl)amino)-1-oxopropan-2-yl)carbamate (I-14e) as an oil. MS: m/z=771.6 [M+H−H₂O].

### Step D—Synthesis of Compound I-14

A mixture of tert-butyl ((2S)-1-(((2S)-1-((4-(1-hydroxy-2-morpholino-2-oxoethyl)phenyl)amino)-1-oxopropan-2-yl)amino)-1-oxopropan-2-yl)carbamate (I-14e, 720 mg, 0.91 mmol), bis(4-nitrophenyl) carbonate (550 mg, 1.8 mmol) and DIPEA (0.16 mL, 0.91 mmol) in DMF (2.3 mL) was stirred at RT for 2 h At this point, additional bis(4-nitrophenyl) carbonate (270 mg, 0.89 mmol) and DIPEA (0.16 mL, 0.91 mmol) were added. After stirring for another 3 h, additional bis(4-nitrophenyl) carbonate (270 mg, 0.89 mmol) and DIPEA (0.16 mL, 0.91 mmol) were added. After 30 minutes, the reaction mixture was directly purified by reverse phase column chromatography (10-90% MeCN/H₂O with 0.1% formic acid modifier) to afford tert-butyl ((2S)-1-(((2S)-1-((4-(5-methyl-1-(4-nitrophenoxy)-1,4-dioxo-2,8,11,14,17,20,23,26,29-nonaoxa-5-azatriacontan-3-yl)phenyl)amino)-1-oxopropan-2-yl)amino)-1-oxopropan-2-yl)carbamate (I-14) as a thick gel. MS: m/z=976 [M+Na].

### Preparation of Intermediate I-15

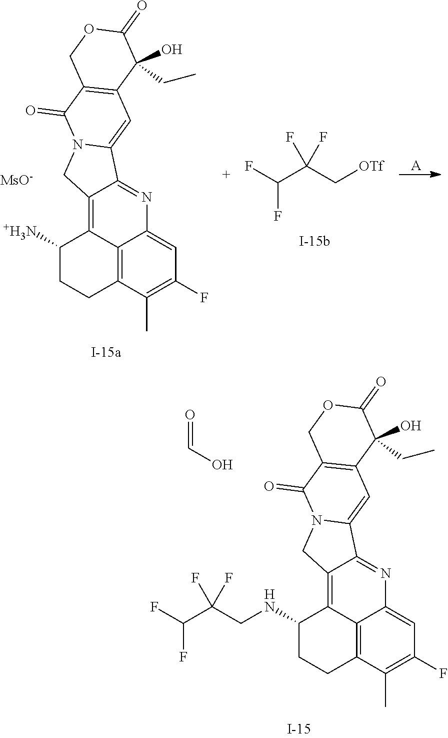

### Step A—Synthesis of Compound I-15

A mixture of 2,2,3,3-tetrafluoropropyl trifluoromethanesulfonate (I-15b, 240 mg, 0.90 mmol), (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (I-15a, 0.43 mL, 0.15-0.17 mmol, ˜0.35-0.40 M in DMF, with 3 eq. TEA), DIPEA (0.052 mL, 0.30 mmol) and DMF (0.6 mL) was stirred at RT for 2.5 days. The mixture was then directly purified via reverse phase column chromatography (10-80% MeCN/water with 0.1% formic acid modifier) to afford (1S,9S)-9-ethyl-5-fluoro-9-hydroxy-4-methyl-1-((2,2,3,3-tetrafluoropropyl)amino)-1,2,3,9,12,15-hexahydro-10H,13H-benzo[de]pyrano[3′,4′:6,7]indolizino[1,2-b]quinoline-10,13-dione formate (I-15). MS: m/z=550 [M+H].

### Intermediates I-16 and I-17

### Preparation of Intermediates I-16 and I-17

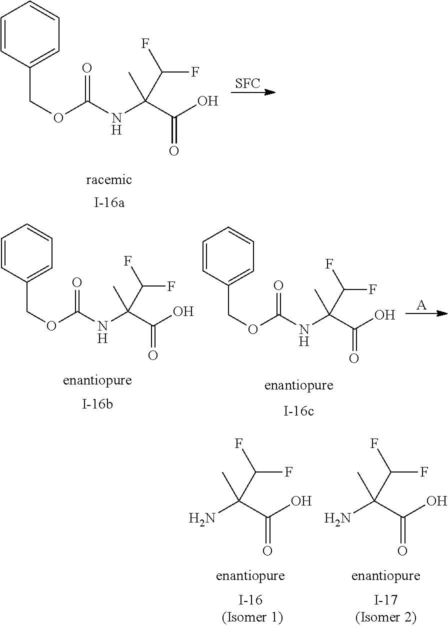

### SFC Separation of Enantiomers to Yield I-16b and I-16c

Racemic 2-(((benzyloxy)carbonyl)amino)-3,3-difluoro-2-methylpropanoic acid (I-16a, 1.2 g, 4.4 mmol) was subjected to preparative chiral SFC (AD-H 2×25 cm, 10% (EtOH+0.1% DEA)/CO₂, 100 bar, 70 mL/min) to afford enantiomer 1 (peak 1, 95% ee, absolute configuration unknown) and enantiomer 2 (peak 2, 99% ee, absolute configuration unknown) as diethylamine salts.

To obtain the free acid, the diethylamine salt of enantiomer 1 was dissolved in EtOAc and shaken with aqueous 1M HCl. The organic layer was back-extracted with EtOAc twice. The combined organic layers were dried over Na₂SO₄, filtered, and concentrated under reduced pressure to afford 2-(((benzyloxy)carbonyl)amino)-3,3-difluoro-2-methylpropanoic acid (I-16b, enantiomer 1, absolute configuration unknown). ¹H NMR (500 MHz, DMSO-d₆) δ 13.20 (s, 1H), 8.10 (s, 1H), 7.42-7.27 (m, 5H), 6.28 (t, J=56.1 Hz, 1H), 5.03 (s, 2H), 1.34 (s, 3H).

The free acid of 2-(((benzyloxy)carbonyl)amino)-3,3-difluoro-2-methylpropanoic acid (I-16c, enantiomer 2, absolute configuration unknown) was obtained in analogous fashion.

### Step A—Synthesis of Intermediates I-16 and I-17

A mixture of 2-(((benzyloxy)carbonyl)amino)-3,3-difluoro-2-methylpropanoic acid (I-16b, enantiomer 1, 290 mg, 1.1 mmol), 10 wt % Pd/C (430 mg, 0.41 mmol Pd) and MeOH (20 mL) was stirred under H₂ (1.0 atm) overnight. The reaction mixture was then filtered through Celite®, rinsing with MeOH and DCM, followed by removal of solvent under reduced pressure to afford 2-amino-3,3-difluoro-2-methylpropanoic acid (I-16, enantiomer 1, absolute configuration unknown) as a solid. ¹H NMR (500 MHz, DMSO-d₆) δ 7.71 (br s, 3H), 6.18 (t, J=54.4 Hz, 1H), 1.27 (s, 3H).

2-Amino-3,3-difluoro-2-methylpropanoic acid (I-17, enantiomer 2, absolute configuration unknown) was obtained in analogous fashion from I-16c.

### Preparation of Intermediate I-18

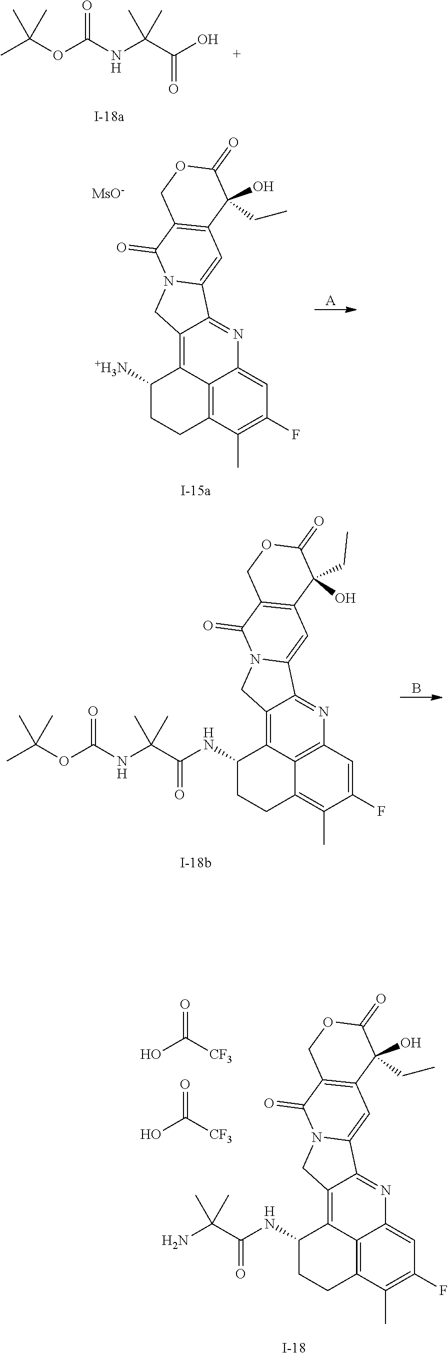

### Step A—Synthesis of Compound I-18b

To a stirred mixture of 2-((tert-butoxycarbonyl)amino)-2-methylpropanoic acid (I-18a, 150 mg, 0.75 mmol), Hunig's Base (87 μL, 0.50 mmol) and (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (I-15a, 1.5 mL, 0.51-0.59 mmol, ˜0.35-0.40 M in DMF with 3 eq. Et₃N) was added HATU (270 mg, 0.70 mmol). The reaction mixture was stirred at RT for 15 min, and then subjected to reverse phase column chromatography (20-100% MeCN/H₂O with 0.1% formic acid modifier) to afford tert-butyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (I-18b) as a solid. MS: m/z=621 [M+H].

### Step B—Synthesis of Compound I-18

A mixture of tert-butyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (I-18b, 250 mg, 0.40 mmol), TFA (0.31 mL, 4.0 mmol) and DCM (1.3 mL) was stirred at RT for 6 h. The mixture was then homogenized via addition of MeOH and concentrated under vacuum to afford crude 2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-methylpropanamide bis(2,2,2-trifluoroacetate) (I-18) as a solid, which was used directly without further purification.

### Intermediate I-19

### Preparation of Intermediate I-19

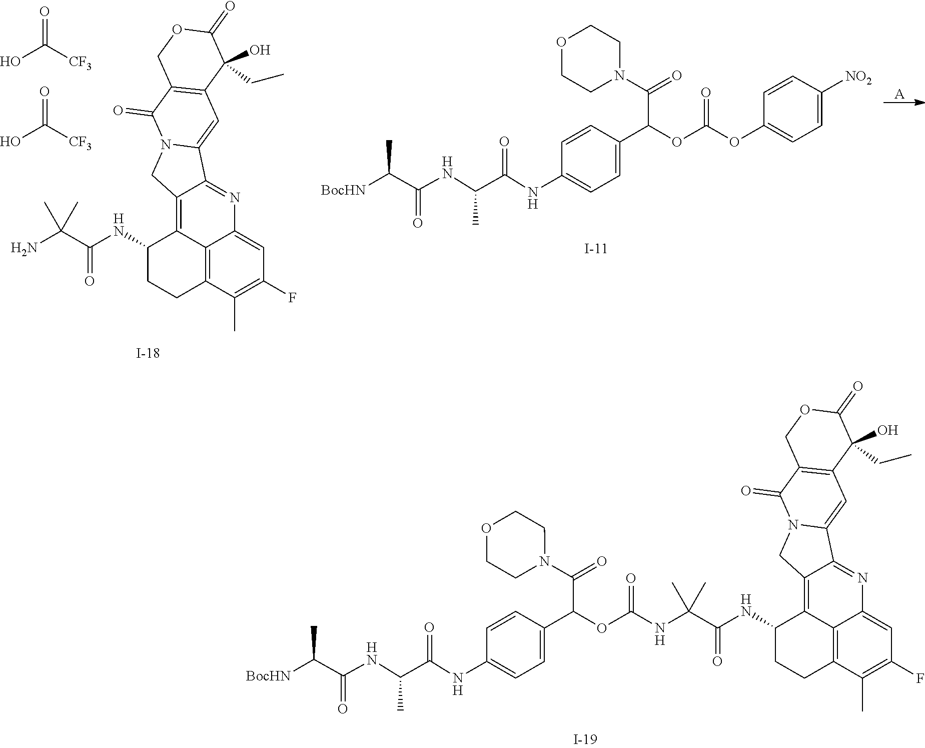

### Step A—Synthesis of Compound I-19

A mixture of 2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-methylpropanamide bis(2,2,2-trifluoroacetate) (I-18, 0.65 mL, 0.065 mmol, 0.1 M in DMF), tert-butyl ((2S)-1-(((2S)-1-((4-(2-morpholino-1-(((4-nitrophenoxy)carbonyl)oxy)-2-oxoethyl)phenyl)amino)-1-oxopropan-2-yl)amino)-1-oxopropan-2-yl)carbamate (I-11, 32 mg, 0.050 mmol), HOAt (6.8 mg, 0.050 mmol), DIPEA (44 μL, 0.25 mmol), 2,6-lutidine (29 μL, 0.25 mmol) and DMF (1.0 mL) was stirred under argon at RT for 3 days, with occasional heating to 40° C. and monitoring reaction progress by LCMS. The mixture was then directly purified via reverse phase column chromatography (10-90% MeCN/water with 0.1% formic acid modifier) to afford 1-(4-((S)-2-((S)-2-((tert-butoxycarbonyl)amino)propanamido)propanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (I-19). MS: m/z=1025 [M+H].

The following compounds of the present disclosure in Table 5 were made using similar methods described in Example I-19 with subtle variations in reaction times and/or solvents, and substituting appropriate reactants and/or reagents:

TABLE 5

| | | MS |
|---|---|---|
| Intermediate | Structure | [M + H] |
| I-20 | 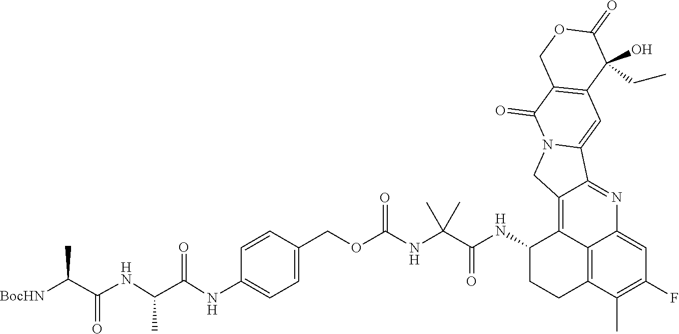 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)OC(C)(C)C)cc1)CC3] | 912 |
| | from I-18 and I-12, at 23-40° C. | |
| I-21 | 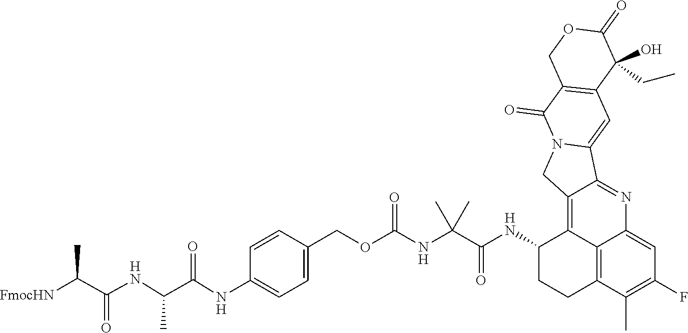 | 1034 |
| | from I-18 and I-13, at 23-40° C. | |
| I-22 | 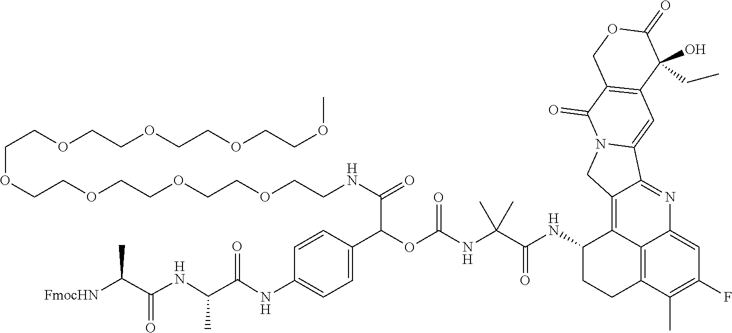 | 733.8 (m/2) [M + H + Na] |
| | from I-18 and I-9, at RT, using HOBt hydrate instead of HOAt | |
| I-23 | 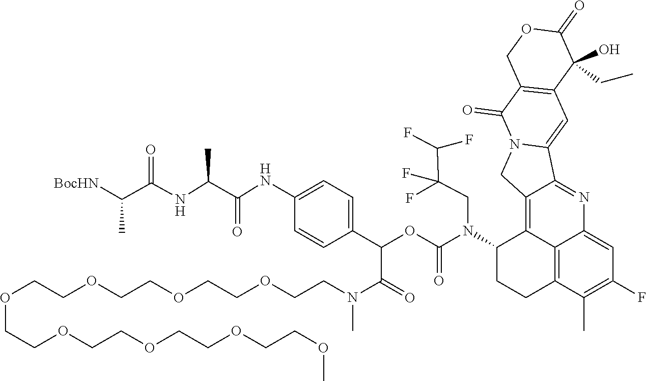 | 633 (m/z) [M + 2H- Boc] |
| | from I-15 and I-14, at 40° C., using DMAP instead of HOAt | |

### Preparation of Intermediate I-27

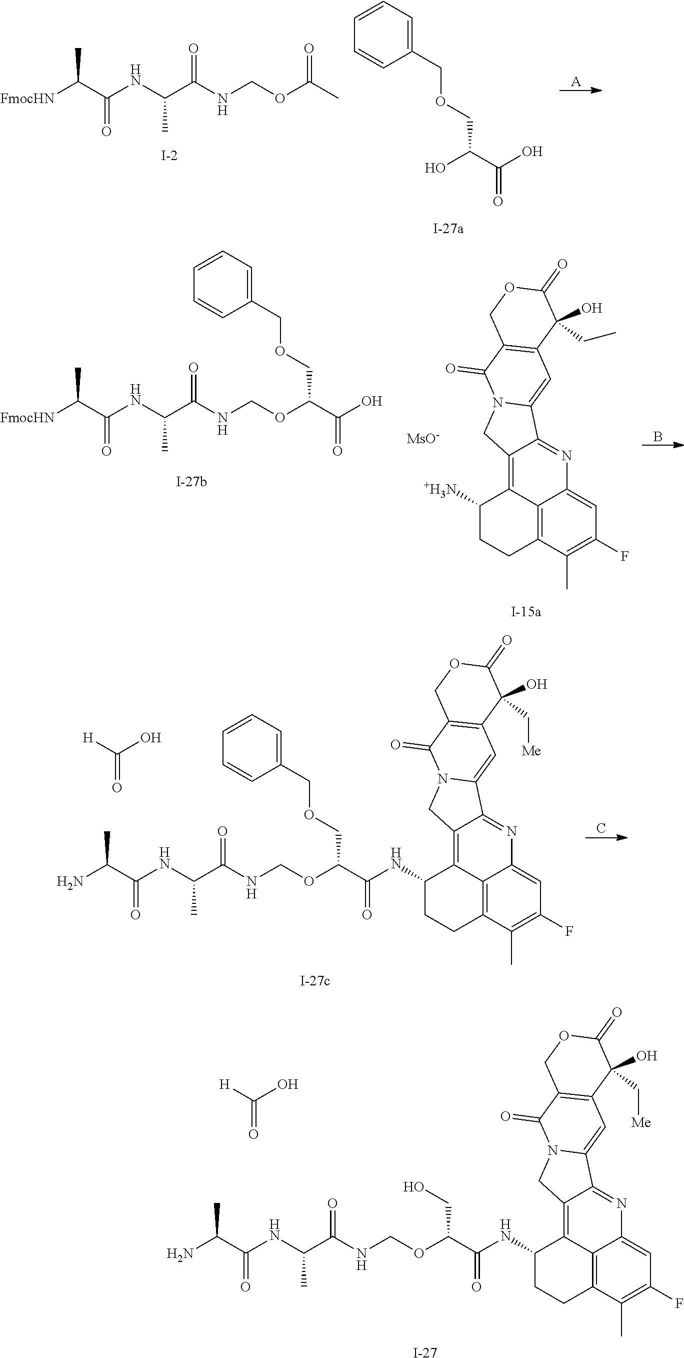

### Step A—Synthesis of Compound I-27b

To a solution of (5S,8S)-1-(9H-fluoren-9-yl)-5,8-dimethyl-3,6,9-trioxo-2-oxa-4,7,10-triazaundecan-11-yl acetate (I-2, 180 mg, 0.40 mmol) and (R)-3-(benzyloxy)-2-hydroxypropanoic acid (I-27a, 520 mg, 2.7 mmol) in DCM (2.0 mL) was added TFA (61 μL, 0.80 mmol). The resulting mixture was stirred at RT for 5 h. The mixture was then concentrated to dryness under reduced pressure, redissolved in DMSO, and purified by reverse phase column chromatography (MeCN/H₂O with 0.1% formic acid modifier) to afford (5S,8S,13R)-13-((benzyloxy)methyl)-1-(9H-fluoren-9-yl)-5,8-dimethyl-3,6,9-trioxo-2,12-dioxa-4,7,10-triazatetradecan-14-oic acid (I-27b) as a solid. MS: m/z=612 [M+Na].

### Step B—Synthesis of Compound I-27c

To a stirred solution of (5S,8S,13R)-13-((benzyloxy)methyl)-1-(9H-fluoren-9-yl)-5,8-dimethyl-3,6,9-trioxo-2,12-dioxa-4,7,10-triazatetradecan-14-oic acid (I-27b, 60 mg, 0.10 mmol) and HATU (46 mg, 0.12 mmol) in DMF (0.30 mL) were added (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (I-15a, 0.33 mL, 0.12-0.13 mmol, ˜0.35-0.40 M in DMF, with 3 eq. TEA) and DIPEA (18 μL, 0.10 mmol). After stirring at RT for 10 minutes, the mixture was treated with 4-methylpiperidine (60 μL, 0.51 mmol) and stirred for another 2 h. The mixture was then quenched with formic acid (20 μL) and purified via reverse phase column chromatography (10-60% MeCN/H₂O with 0.1% formic acid modifier) to afford (R)-2-(((S)-2-((S)-2-aminopropanamido)propanamido)methoxy)-3-(benzyloxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propanamide formate (I-27c) as a solid. MS: m/z=785 [M+H].

### Step C—Synthesis of Compound I-27

A mixture of (R)-2-(((S)-2-((S)-2-aminopropanamido)propanamido)methoxy)-3-(benzyloxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propanamide formate (I-27c, 31 mg, 0.037 mmol) and 10 wt % Pd/C (8.0 mg, 0.0075 mmol Pd) in MeOH (1.0 mL) was stirred under H₂ (1.0 atm) at RT. After 3 h, DCM (1.0 mL) was added and stirring under H₂ was continued for 3 h. At that point, the reaction mixture was concentrated to dryness under reduced pressure and the residue was redissolved in DMSO (1.0 mL) and MeOH (1.0 mL). Another portion of 10 wt % Pd/C was added, and the reaction mixture was stirred under H₂ (1.0 atm) until reaction progress stopped, as assessed by LCMS. The mixture was then syringe-filtered and directly purified by reverse phase column chromatography (10-60% MeCN/H₂O with 0.1% formic acid modifier) to afford (R)-2-(((S)-2-((S)-2-aminopropanamido)propanamido)methoxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropanamide formate (I-27). MS: m/z=695 [M+H].

### Preparation of Intermediate I-28

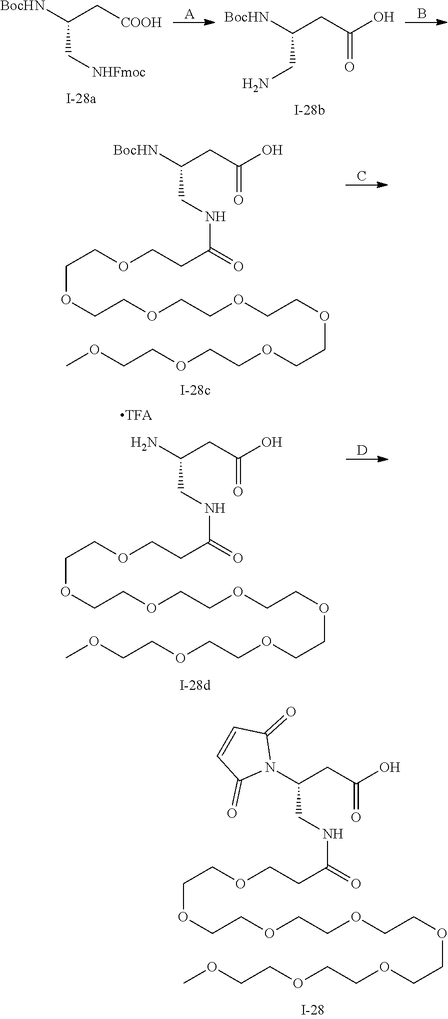

### Step A—Synthesis of Compound I-28b

To a solution of (R)-4-((((9H-fluoren-9-yl)methoxy)carbonyl)amino)-3-((tert-butoxycarbonyl)amino)butanoic acid (I-28a, 200 mg, 0.45 mmol) in DCM (3.0 mL) was added diethylamine (0.14 mL, 1.4 mmol). The reaction was stirred at RT overnight. The mixture was concentrated under reduced pressure, and the resulting crude (R)-4-amino-3-((tert-butoxycarbonyl)amino)butanoic acid (I-28b) was used directly in the next reaction.

### Step B—Synthesis of Compound I-28c

To a solution of (R)-4-amino-3-((tert-butoxycarbonyl)amino)butanoic acid (I-28b, 0.099 g, 0.45 mmol) and N-methylmorpholine (0.050 mL, 0.45 mmol) in DMF (3.0 mL) was added 2,5-dioxopyrrolidin-1-yl 2,5,8,11,14,17,20,23-octaoxahexacosan-26-oate (0.23 g, 0.45 mmol). The reaction was stirred at RT for ˜3 hrs. With some MeOH, the mixture was transferred to a new vial, and concentrated under reduced pressure. The remaining material was washed with ether. The resulting crude (R)-29-((tert-butoxycarbonyl)amino)-26-oxo-2,5,8,11,14,17,20,23-octaoxa-27-azahentriacontan-31-oic acid (I-28c) was dried under reduced pressure and used directly in the next reaction.

### Step C—Synthesis of Compound I-28d

To a mixture of (R)-29-((tert-butoxycarbonyl)amino)-26-oxo-2,5,8,11,14,17,20,23-octaoxa-27-azahentriacontan-31-oic acid (I-28c, 0.28 g, 0.45 mmol) and DCM (5.0 mL) was added TFA (0.15 mL, 1.9 mmol). The reaction was stirred at RT overnight. Then, another portion of TFA was added, and the reaction was stirred at RT for ˜5.5 hrs. The mixture was concentrated under reduced pressure, and the resulting (R)-29-amino-26-oxo-2,5,8,11,14,17,20,23-octaoxa-27-azahentriacontan-31-oic acid, TFA (I-28d) was used crude directly in the next reaction.

### Step D—Synthesis of Compound I-28

To a mixture of (R)-29-amino-26-oxo-2,5,8,11,14,17,20,23-octaoxa-27-azahentriacontan-31-oic acid, TFA (I-28d, 280 mg, 0.45 mmol) and maleic anhydride (45 mg, 0.45 mmol) was added absolute ethanol (3.0 mL), followed by triethylamine (0.20 mL, 1.4 mmol). The mixture was stirred at RT for ˜4 hr 20 min, before another portion of maleic anhydride (14 mg) was added. The reaction was then stirred at RT overnight. The solution was concentrated under reduced pressure, and then acetic anhydride (2.0 mL, 21 mmol) was added, followed by sodium acetate (75 mg, 0.91 mmol). The reaction was heated to 65° C. for ˜2 hr 20 min. The material was cooled to RT, then 1M HCl (0.50 mL) was added, and the material was then directly subjected to silica gel flash column chromatography (0-100% MeOH/DCM) to afford (R)-29-(2,5-dioxo-2,5-dihydro-1H-pyrrol-1-yl)-26-oxo-2,5,8,11,14,17,20,23-octaoxa-27-azahentriacontan-31-oic acid (I-28). MS: m/z=593.6 [M+H].

### Preparation of Intermediate I-29

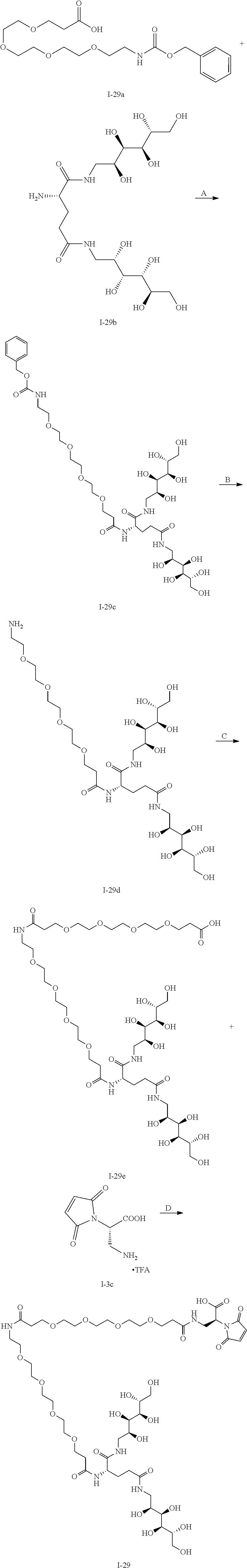

### Step A—Synthesis of Compound I-29c

Cbz-N-amido-PEG4-acid (I-29a, 300 mg, 0.76 mmol) was added to a vial with DMF (13 mL), HATU (240 mg, 0.63 mmol), (S)-2-amino-N¹,N⁵-bis((2S,3R,4R,5R)-2,3,4,5,6-pentahydroxyhexyl)pentanediamide (I-29b, 300 mg, 0.63 mmol) and Hunig's Base (0.11 mL, 0.63 mmol). The reaction was stirred overnight at room temperature and then concentrated under reduced pressure. The resulting residue was purified via C18 flash column chromatography (0-50% MeCN in water) to afford benzyl ((17S,23S,24R,25R,26R)-23,24,25,26,27-pentahydroxy-15,20-dioxo-17-(((2S,3R,4R,5R)-2,3,4,5,6-pentahydroxyhexyl)carbamoyl)-3,6,9,12-tetraoxa-16,21-diazaheptacosyl)carbamate (I-29c). MS: m/z=855.6 [M+H].

### Step B—Synthesis of Compound I-29d

benzyl ((17S,23S,24R,25R,26R)-23,24,25,26,27-pentahydroxy-15,20-dioxo-17-(((2S,3R,4R,5R)-2,3,4,5,6-pentahydroxyhexyl)carbamoyl)-3,6,9,12-tetraoxa-16,21-diazaheptacosyl)carbamate (I-29c, 1.0 g, 1.2 mmol) was added to a flask containing water (3.9 mL) and EtOH (20 mL). Palladium on carbon (0.12 g, 1.2 mmol) was added, followed by triethylsilane (1.9 mL, 12 mmol) slowly (with evolution of gas). The resulting emulsion was stirred at RT for 1 hr before being filtered and washed with EtOH. The filtrate was concentrated under reduced pressure and the resulting (S)-2-(1-amino-3,6,9,12-tetraoxapentadecan-15-amido)-N¹,N⁵-bis((2S,3R,4R,5R)-2,3,4,5,6-pentahydroxyhexyl)pentanediamide (I-29d) was used directly in the next reaction.

### Step C—Synthesis of Compound I-29e

(S)-2-(1-amino-3,6,9,12-tetraoxapentadecan-15-amido)-N¹,N⁵-bis((2S,3R,4R,5R)-2,3,4,5,6-pentahydroxyhexyl)pentanediamide (I-29d, 200 mg, 0.28 mmol) was added to a vial with DMF (2.0 mL), N-ethyl-N-isopropylpropan-2-amine (72 mg, 0.56 mmol) and 16-((2,5-dioxopyrrolidin-1-yl)oxy)-16-oxo-4,7,10,13-tetraoxahexadecanoic acid (110 mg, 0.28 mmol). The reaction was stirred at RT for 2 hr and then purified via C18 flash column chromatography (0-40% MeCN in water with 0.1% TFA modifier) to afford (34S,40S,41R,42R,43R)-40,41,42,43,44-pentahydroxy-16,32,37-trioxo-34-(((2S,3R,4R,5R)-2,3,4,5,6-pentahydroxyhexyl)carbamoyl)-4,7,10,13,20,23,26,29-octaoxa-17,33,38-triazatetratetracontanoic acid (I-29e). MS: m/z=997.4 [M+H].

### Step D—Synthesis of Compound I-29

Following a similar HATU protocol to that described in step A using I-29e and I-3c, with purification by C18 flash column chromatography (0-60% MeCN in water), afforded (2S,38S,44S,45R,46R,47R)-2-(2,5-dioxo-2,5-dihydro-1H-pyrrol-1-yl)-44,45,46,47,48-pentahydroxy-5,20,36,41-tetraoxo-38-(((2S,3R,4R,5R)-2,3,4,5,6-pentahydroxyhexyl)carbamoyl)-8,11,14,17,24,27,30,33-octaoxa-4,21,37,42-tetraazaoctatetracontanoic acid (I-29) as a solid. MS: m/z=1164.6 [M+H].

One of ordinary skill in the art can appreciate how to make other PEG-sugar intermediates by following the procedure for making Intermediate-29 with modifications where necessary.

### Example 1

### Preparation of Example 1

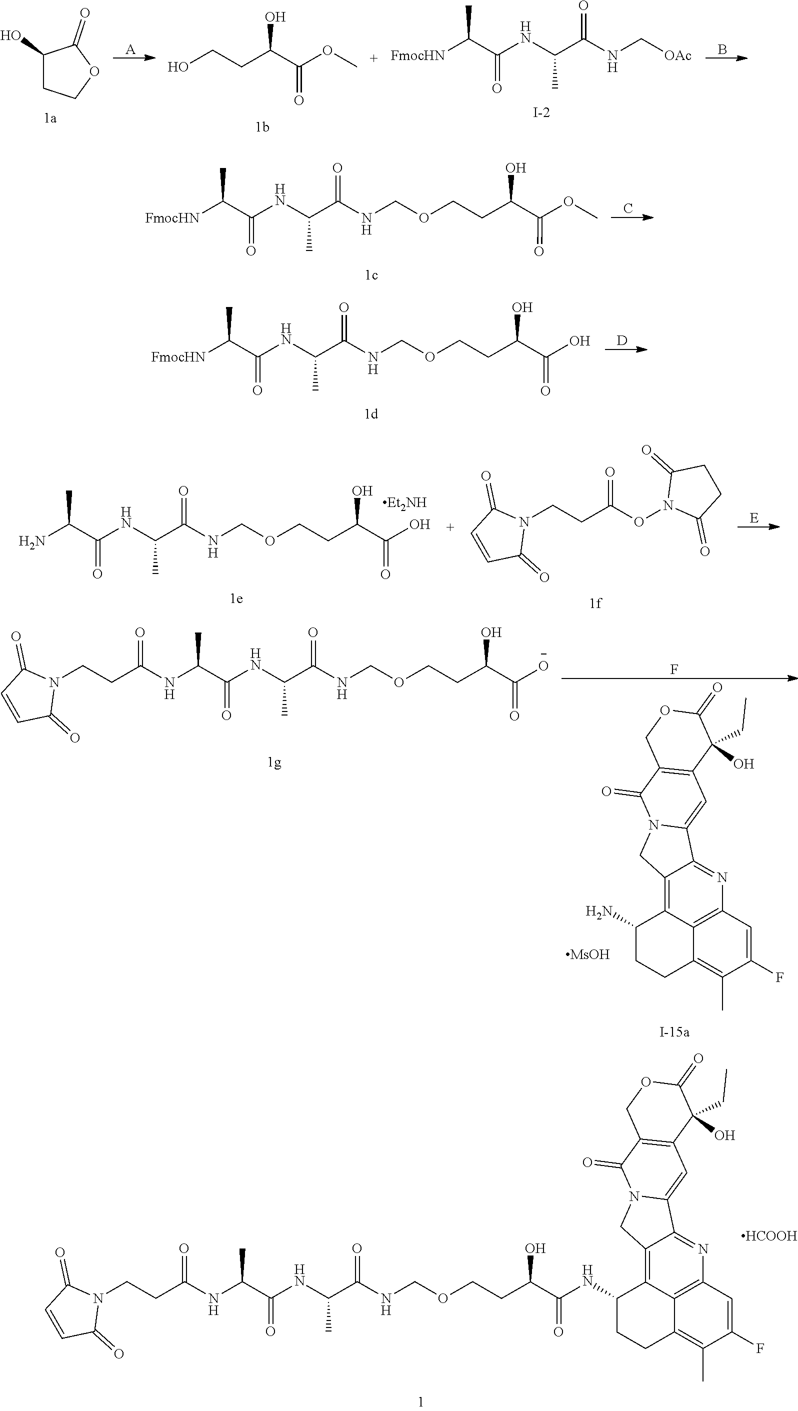

### Step A—Synthesis of Compound 1b

To a solution of (R)-3-hydroxydihydrofuran-2(3H)-one (1a, 2.2 g, 21.6 mmol) in MeOH (20 mL) was added Dowex 50W X8, hydrogen form, strongly acidic, 200-400 mesh resin (1.1 g). The reaction was stirred at RT for 2 h 20 min. The mixture was filtered over Celite, and the filtrate was concentrated under reduced pressure. The resulting methyl (R)-2,4-dihydroxybutanoate (1b) was used directly in the next reaction as an oil.

### Step B—Synthesis of Compound 1c

To a solution of methyl (R)-2,4-dihydroxybutanoate (1b, 3.1 g, 23 mmol) and (5S,8S)-1-(9H-fluoren-9-yl)-5,8-dimethyl-3,6,9-trioxo-2-oxa-4,7,10-triazaundecan-11-yl acetate (I-2, 1.5 g, 3.3 mmol) in DCM (15 mL) was added trifluoroacetic acid (0.80 mL, 10 mmol). The reaction was stirred at RT for ˜2.5 h (monitored by LCMS). The solution was directly subjected to silica gel flash column chromatography (0-10% MeOH/DCM) to afford methyl (5S,8S,15R)-1-(9H-fluoren-9-yl)-15-hydroxy-5,8-dimethyl-3,6,9-trioxo-2,12-dioxa-4,7,10-triazahexadecan-16-oate (1c) as a solid. MS: m/z=550 [M+Na].

### Step C—Synthesis of Compound 1d

To a solution of methyl (5S,8S,15R)-1-(9H-fluoren-9-yl)-15-hydroxy-5,8-dimethyl-3,6,9-trioxo-2,12-dioxa-4,7,10-triazahexadecan-16-oate (1c, 0.41 g, 0.79 mmol) in DMSO (10 mL) was added Novozym 51032 (5.0 mL) enzyme solution in 0.1 M K₃PO₄ pH 8 buffer (100 mL). The reaction was heated to 30° C. overnight. The mixture was then cooled, diluted with 3:1 CHCl₃:IPA (100 mL), and 1M HCl (12 mL). After extraction, the mixture was passed through a hydrophobic membrane phase separator. The aqueous layer was washed with the 3:1 CHCl₃:IPA mixture again (100 mL), and that mixture was passed through a phase separator. The combined organic layers were passed through a phase separator again and concentrated under reduced pressure. The crude material was subjected to column chromatography (0-60% MeOH/DCM, 100% MeOH flush) to afford (5S,8S,15R)-1-(9H-fluoren-9-yl)-15-hydroxy-5,8-dimethyl-3,6,9-trioxo-2,12-dioxa-4,7,10-triazahexadecan-16-oic acid (1d) as a solid. MS: m/z=536 [M+Na].

### Step D—Synthesis of Compound 1e

To a solution of (5S,8S,15R)-1-(9H-fluoren-9-yl)-15-hydroxy-5,8-dimethyl-3,6,9-trioxo-2,12-dioxa-4,7,10-triazahexadecan-16-oic acid (1d, 0.28 g, 0.55 mmol) in DMF (6.0 mL) was added diethylamine (0.18 mL, 1.7 mmol). The reaction was stirred at RT for ˜1 h 45 min. The mixture was concentrated under reduced pressure to remove excess diethylamine. The resulting (R)-4-(((S)-2-((S)-2-aminopropanamido)propanamido)methoxy)-2-hydroxybutanoic acid diethylamine salt (1e) solution was directly used crude for the next reaction.

### Step E—Synthesis of Compound 1g

To a solution of (R)-4-(((S)-2-((S)-2-aminopropanamido)propanamido)methoxy)-2-hydroxybutanoic acid, diethylamine salt (1e, 0.044 g, 0.15 mmol) in DMF (1.0 mL) was added N-methylmorpholine (0.020 mL, 0.18 mmol), followed by 2,5-dioxopyrrolidin-1-yl 3-(2,5-dioxo-2,5-dihydro-1H-pyrrol-1-yl)propanoate (1f, 0.040 g, 0.15 mmol). The reaction was stirred at RT for ˜1 h 45 min. The solution was then concentrated under reduced pressure. The residue was washed with ether 3×. The resulting (5S,8S,15R)-1-(2,5-dioxo-2,5-dihydro-1H-pyrrol-1-yl)-15-hydroxy-5,8-dimethyl-3,6,9-trioxo-12-oxa-4,7,10-triazahexadecan-16-oate (1g) was dried under reduced pressure and used directly in the next reaction.

### Step F—Synthesis of Compound 1

To a solution of (5S,8S,15R)-1-(2,5-dioxo-2,5-dihydro-1H-pyrrol-1-yl)-15-hydroxy-5,8-dimethyl-3,6,9-trioxo-12-oxa-4,7,10-triazahexadecan-16-oate (1g, 66 mg, 0.15 mmol) and N-methylmorpholine (0.020 mL, 0.18 mmol) in DMF (1.0 mL) was added HATU (57 mg, 0.15 mmol). The reaction was stirred at RT for ˜5 min before (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (I-15a, 0.30 mL, 0.11-0.12 mmol, −0.35-0.40 M in DMF with 3.0 eq. Et₃N) solution was added. The reaction was stirred at RT for ˜4 h. The reaction was filtered and then purified by reverse phase column chromatography (25-65% MeCN/water with 0.1% FA modifier) to afford (R)-4-(((S)-2-((S)-2-(3-(2,5-dioxo-2,5-dihydro-1H-pyrrol-1-yl)propanamido)propanamido)propanamido)methoxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxybutanamide, formic acid (1) as an oil. MS: m/z=860 [M+H]. ¹H NMR (500 MHz, DMSO-d₆) δ 8.56 (t, J=6.5 Hz, 1H), 8.49 (d, J=9.0 Hz, 1H), 8.30 (s, 2H), 8.18 (d, J=7.2 Hz, 1H), 8.01 (d, J=7.4 Hz, 1H), 7.77 (d, J=10.9 Hz, 1H), 7.30 (s, 1H), 6.99 (s, 2H), 5.59-5.52 (m, 1H), 5.46-5.37 (m, 2H), 5.22 (d, J=18.9 Hz, 1H), 5.11 (d, J=18.8 Hz, 1H), 4.61-4.55 (m, 1H), 4.54-4.47 (m, 1H), 4.26-4.18 (m, 2H), 4.05 (dd, J=9.2, 3.4 Hz, 1H), 3.61-3.57 (m, 3H, overlapping with water signal), 3.54-3.50 (m, 3H, overlapping with water signal), 3.26-3.18 (m, 2H, overlapping with water signal), 3.17-3.09 (m, 2H, overlapping with water signal), 2.89 (s, 1H), 2.73 (s, 1H), 2.41-2.34 (m, 5H), 2.21-2.12 (m, 2H), 2.08-1.99 (m, 1H), 1.91-1.78 (m, 3H), 1.23 (d, J=7.1 Hz, 3H), 1.15 (d, J=7.1 Hz, 3H), 0.87 (t, J=7.3 Hz, 3H).

The following compounds of the present disclosure in Table 6 were made using similar methods described in Example 1, with subtle variations in reaction times and substituting the appropriate reactants and/or reagents (I-2 and/or 1f (with other appropriate commercially available NHS esters)):

TABLE 6

| Ex- | | MS |
|---|---|---|
| ample | Structure | [M + H] |
| 2 | 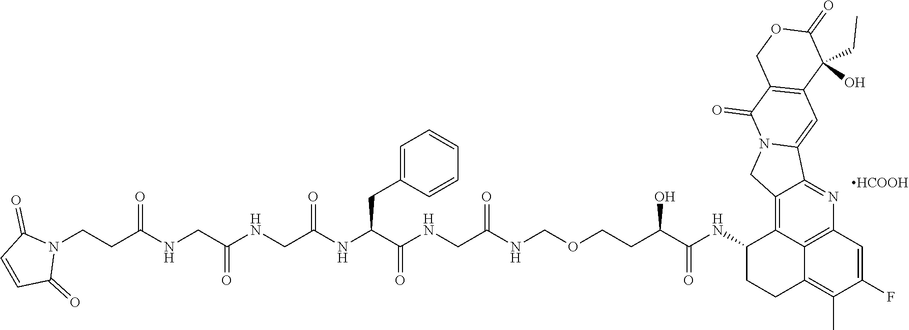 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)CCN1C(=O)C=CC1=O)CC3 ; "·HCOOH"] | 1036 |
| | From I-1 | |
| 3 | 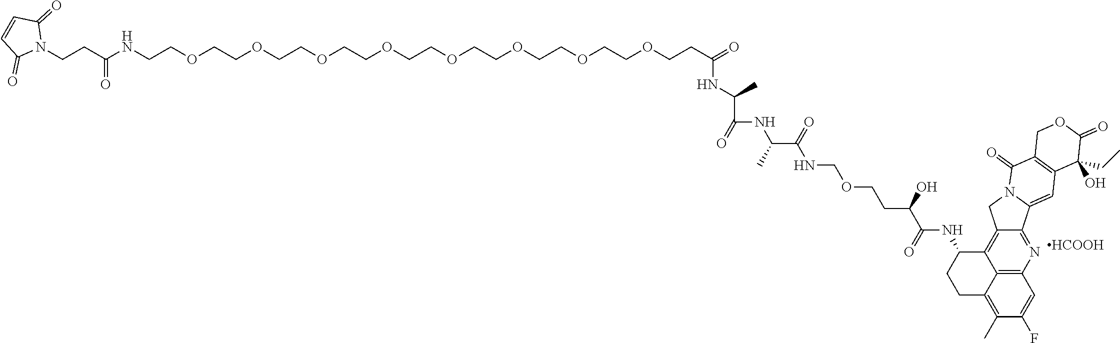 | 1305.4 [M + Na] |
| 4 | 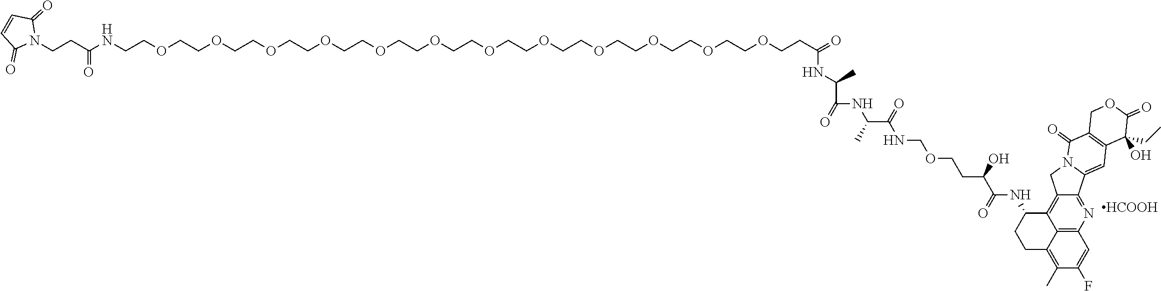 | 730 [M + 2H] (m/2) |
| 5 | 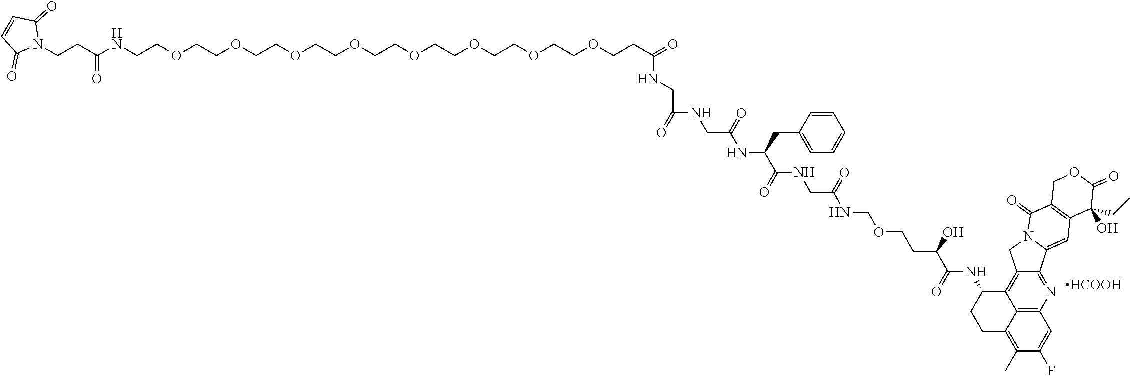 | 1481.4 [M + Na] |
| | From I-1 | |
| 6 | 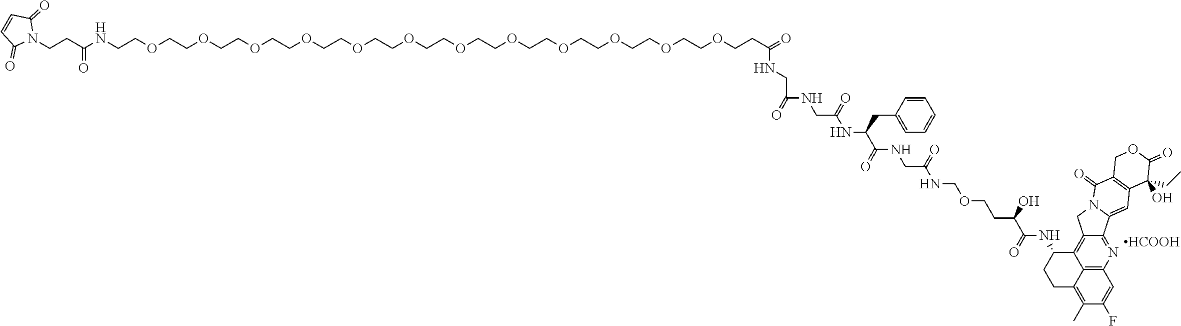 | 1635.7 |
| | From I-1 | |
| 7 | 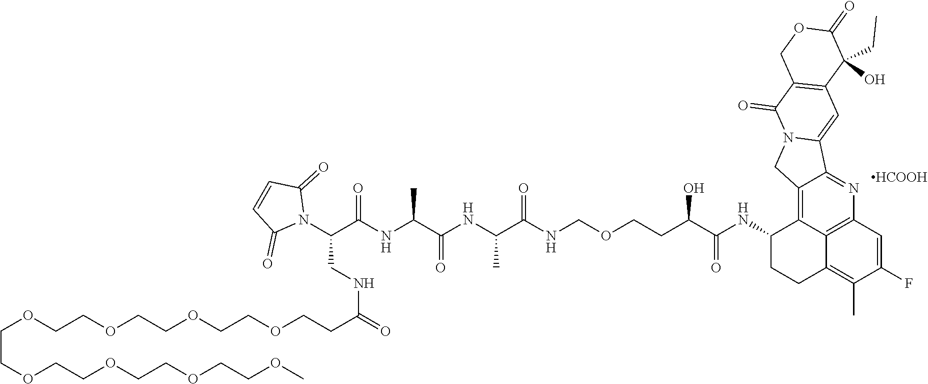 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)[C@H](CNC(=O)CCOCCOCCOCCOCCOCCOCCOCCOC)N1C(=O)C=CC1=O)CC3 ; "·HCOOH"] | 1269.6 |
| | From I-3, and using step A in I-6 (replacing I-6a with 1e) instead of step E in Example 1 | |
| 8 | 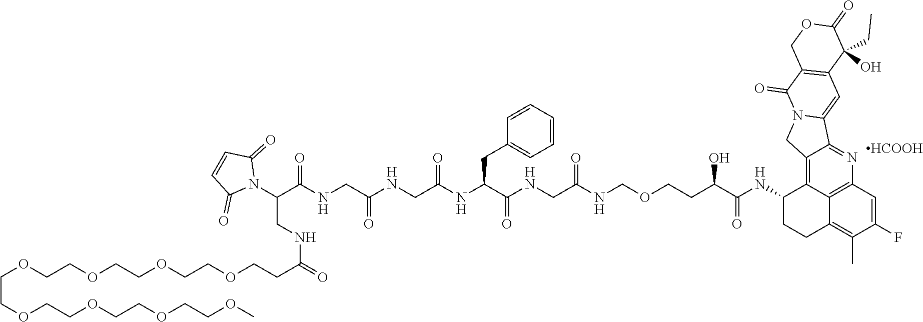 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)C(CNC(=O)CCOCCOCCOCCOCCOCCOCCOCCOC)N1C(=O)C=CC1=O)CC3 ; "·HCOOH"] | 1445.4 |
| | From I-1 and I-3, and using step A in I-6 instead of step E in Example 1; Assumed ~4:1 mixture of diastereomers (adjacent to maleimide) by NMR | |
| 9 | 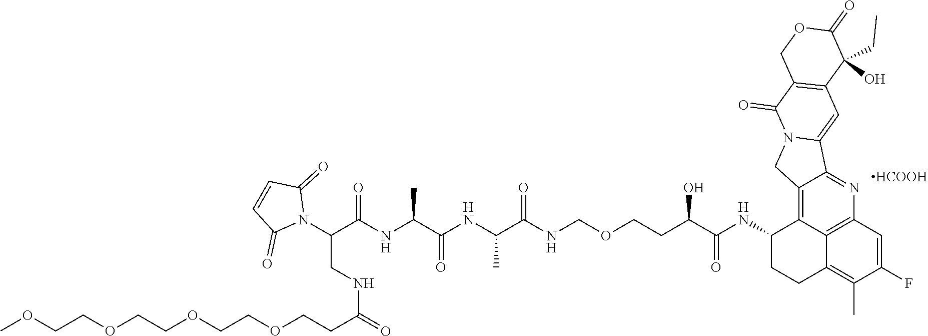 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C(CNC(=O)CCOCCOCCOCCOC)N1C(=O)C=CC1=O)CC3 ; "·HCOOH"] | 1115 [M + Na] |
| | From I-4, and using step A in I-6 (replacing I-6a with 1e) instead of step E in Example 1. Assumed ~3:1 mixture of diastereomers (adjacent to | |
| | maleimide) by NMR | |
| 10 | 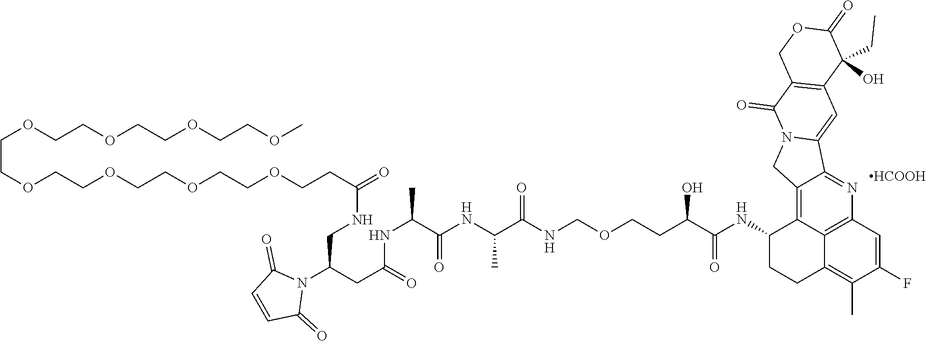 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C[C@H](CNC(=O)CCOCCOCCOCCOCCOCCOCCOCCOC)N1C(=O)C=CC1=O)CC3 ; "·HCOOH"] | 1305 [M + Na] |
| | From I-28, and using step A in I-6 (replacing I-6a with 1e) instead of step E in Example 1 | |
| 11 | 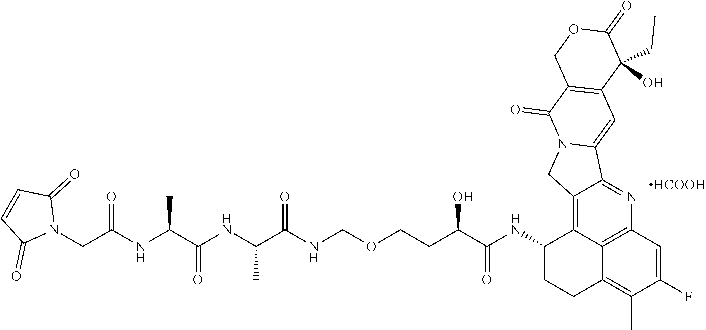 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)CN1C(=O)C=CC1=O)CC3 ; "·HCOOH"] | 868 [M + Na] |
| 12 | 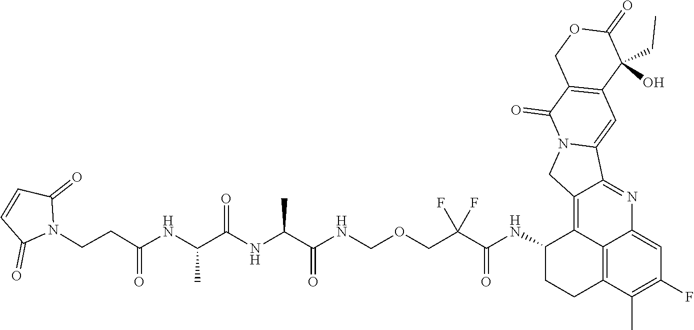 [SMILES: CC[C@@]1(O)C(=O)OCc2c1cc1n(c2=O)Cc2c-1nc1cc(F)c(C)c3c1c2[C@@H](NC(=O)C(F)(F)COCNC(=O)[C@H](C)NC(=O)[C@H](C)NC(=O)CCN1C(=O)C=CC1=O)CC3] | 866 |
| | From 2,2-difluoro-3-hydroxypropanoic acid instead of 1b, skipping step C in Example 1 | |

### Example 13

### Preparation of Example 13

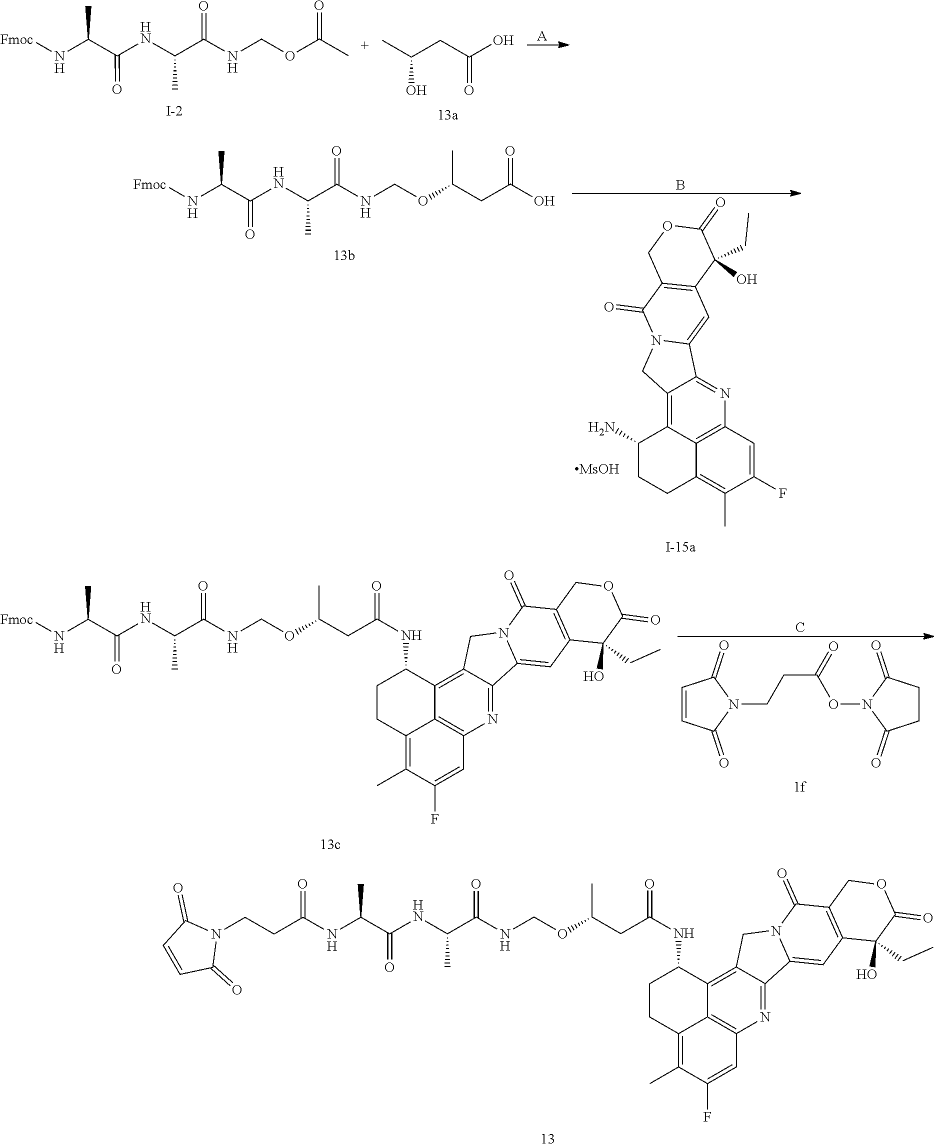

### Step A—Synthesis of Compound 13b

To a solution of (5S,8S)-1-(9H-fluoren-9-yl)-5,8-dimethyl-3,6,9-trioxo-2-oxa-4,7,10-triazaundecan-11-yl acetate (I-2, 200 mg, 0.44 mmol) and (R)-3-hydroxybutanoic acid (13a, 180 mg, 1.8 mmol) in DCM (2.2 mL) was added TFA (68 μl, 0.88 mmol). The resulting mixture was stirred at RT overnight, then purified by silica gel flash column chromatography (0-10% MeOH/DCM) to afford (5S,8S,13R)-1-(9H-fluoren-9-yl)-5,8,13-trimethyl-3,6,9-trioxo-2,12-dioxa-4,7,10-triazapentadecan-15-oic acid (13b). MS: m/z=520 [M+Na].

### Step B—Synthesis of Compound 13c

To a solution of (5S,8S,13R)-1-(9H-fluoren-9-yl)-5,8,13-trimethyl-3,6,9-trioxo-2,12-dioxa-4,7,10-triazapentadecan-15-oic acid (13b, 200 mg, 0.40 mmol) in DMF (2.7 mL) was added (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (I-15a, 1.5 mL, 0.53-0.60 mmol, ˜0.35-0.40 M in DMF with 3.0 eq. Et₃N), HATU (200 mg, 0.52 mmol), and DIPEA (210 μl, 1.2 mmol). The resulting reaction mixture was stirred at RT for 1 h, then purified by reverse phase flash column chromatography (20-95% MeCN/water with 0.1% formic acid) to afford (9H-fluoren-9-yl)methyl ((S)-1-(((S)-1-(((((R)-4-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)amino)-1-oxopropan-2-yl)carbamate (13c). MS: m/z=915 [M+H].

### Step C—Synthesis of Compound 13

To a solution of (9H-fluoren-9-yl)methyl ((S)-1-(((S)-1-(((((R)-4-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)amino)-1-oxopropan-2-yl)carbamate (13c, 60 mg, 0.066 mmol) in DMF (1.0 mL) was added 4-methylpiperidine (1.6 μl, 0.013 mmol), and the reaction was stirred for 30 min at 40° C. The reaction mixture was then cooled to RT, and 2,5-dioxopyrrolidin-1-yl 3-(2,5-dioxo-2,5-dihydro-1H-pyrrol-1-yl)propanoate (1f, 21 mg, 0.079 mmol) was added. The resulting mixture was stirred at RT for 10 min, and then purified by reverse phase column chromatography (20-60% MeCN/water with 0.1% formic acid) to afford (R)-3-(((R)-2-((R)-2-(3-(2,5-dioxo-2,5-dihydro-1H-pyrrol-1-yl)propanamido)propanamido)propanamido)methoxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)butanamide (13). MS: m/z=844 [M+H]. ¹H NMR (500 MHz, DMSO-d₆) δ 8.53 (t, J=6.5 Hz, 1H), 8.43 (d, J=8.7 Hz, 1H), 8.32 (s, 1H), 8.17 (d, J=7.2 Hz, 1H), 7.98 (d, J=7.4 Hz, 1H), 7.79 (d, J=10.9 Hz, 1H), 7.31 (s, 1H), 7.00 (s, 2H), 5.58-5.53 (m, 1H), 5.42 (s, 1H), 5.20 (q, J=18.8 Hz, 2H), 4.59 (dd, J=10.2, 6.9 Hz, 1H), 4.48 (dd, J=10.2, 6.3 Hz, 1H), 4.23-4.12 (m, 2H), 4.03-3.95 (m, 1H), 3.61-3.56 (m, 1H), 3.21-3.13 (m, 1H), 2.46-2.34 (m, 6H), 2.22 (dd, J=14.1, 6.3 Hz, 1H), 2.17-2.08 (m, 2H), 1.93-1.80 (m, 2H), 1.20 (d, J=7.1 Hz, 3H), 1.15-1.10 (m, 5H), 0.87 (t, J=7.3 Hz, 3H).

The following compounds of the present disclosure in Table 7 were made using similar methods described in Example 13, with subtle variations in reaction times and substituting the appropriate reactants and/or reagents (I-2, 13a (with other commercially available acids), and/or 1f (with other appropriate commercially available NHS esters)):

TABLE 7

| Ex- | | MS |
|---|---|---|
| ample | Structure | [M + H] |
| 14 | 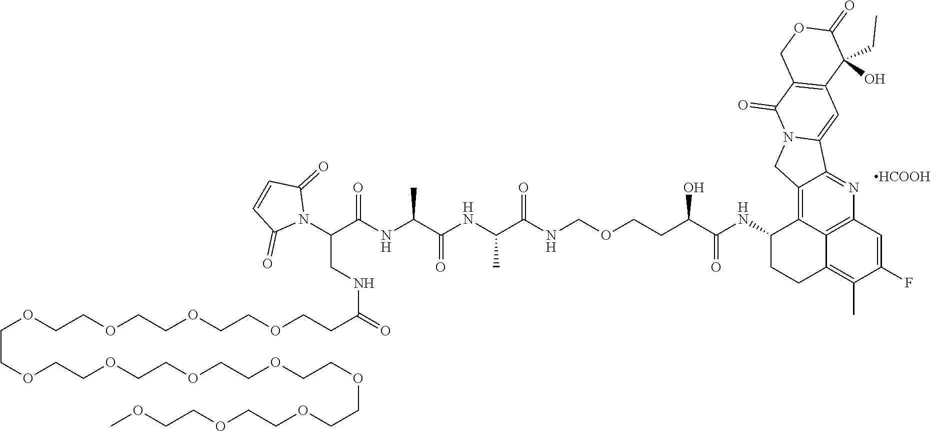 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C(CNC(=O)CCOCCOCCOCCOCCOCCOCCOCCOCCOCCOCCOCCOC)N1C(=O)C=CC1=O)CC3 ; "·HCOOH"] | 1445 |
| | From I-5, and using conditions similar to step A in I-5 instead of the conditions in step C in Example 13 once Fmoc removed; Assumed ~1.3:1 | |
| | mixture of diastereomers (adjacent to maleimide) by NMR | |
| 15 | 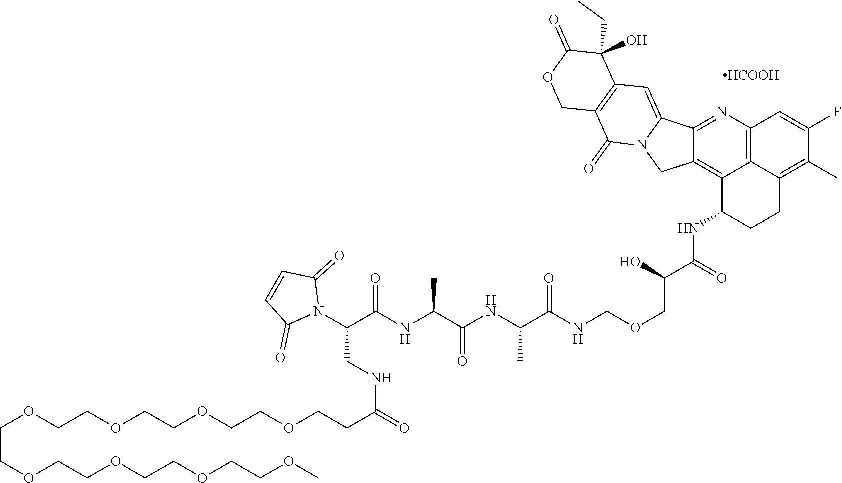 [SMILES: CC[C@@]1(O)C(=O)OCc2c1cc1n(c2=O)Cc2c-1nc1cc(F)c(C)c3c1c2[C@@H](NC(=O)[C@H](O)COCNC(=O)[C@H](C)NC(=O)[C@H](C)NC(=O)[C@H](CNC(=O)CCOCCOCCOCCOCCOCCOCCOCCOC)N1C(=O)C=CC1=O)CC3 ; "·HCOOH"] | 1255.6 |
| | From methyl (R)-2,3-dihydroxypropanoate instead of 13a, then using the conditions of step C in Example 1 to reveal free acid, | |
| | resuming with step B in Example 13, then using conditions similar to step A in I-6 instead of the conditions in step C in | |
| | Example 13 once Fmoc removed | |
| 16 | 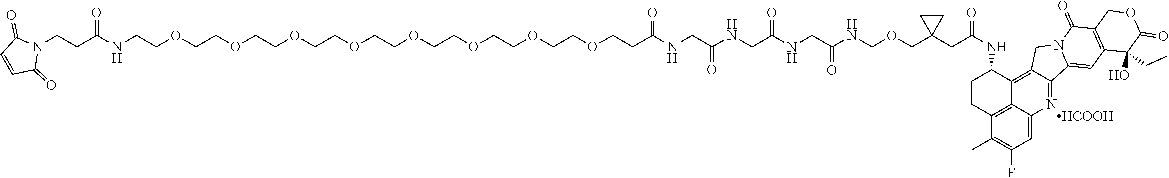 | 1344.5 |
| | From I-7 and 2-(1-(hydroxymethyl)cyclopropyl)acetic acid instead of 13a | |
| 17 | 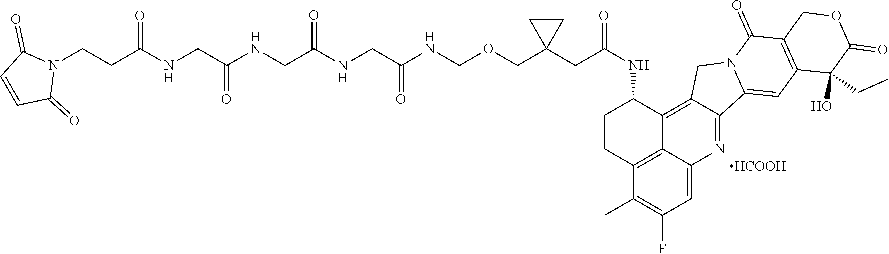 | 921 |
| | From I-7 and 2-(1-(hydroxymethyl)cyclopropyl)acetic acid instead of 13a | |
| 18 | 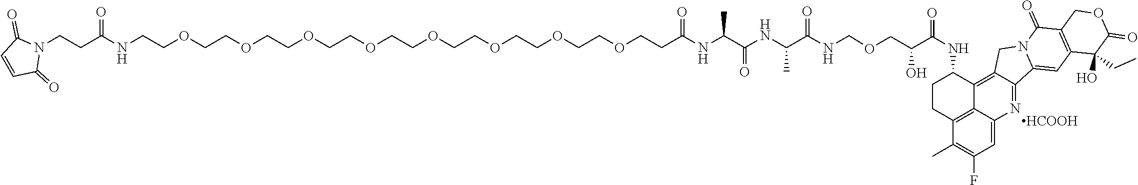 | 1291.6 [M + Na] |
| | From methyl (R)-2,3-dihydroxypropanoate instead of 13a, then using the conditions of step C in Example 1 to reveal free acid, resuming with the steps in Example 13 | |
| 19 | 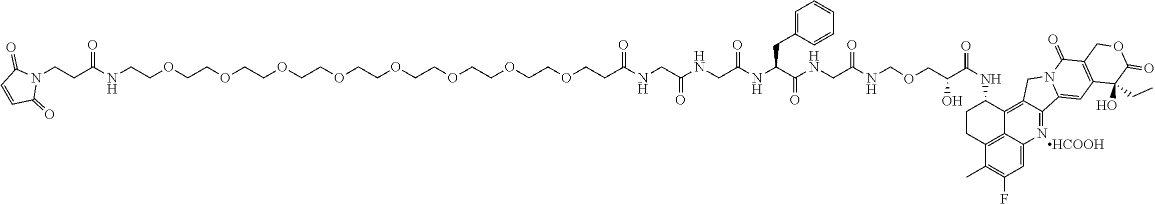 | 1467.5 [M + Na] |
| | From I-1, from methyl (R)-2,3-dihydroxypropanoate instead of 13a, then using the conditions of step C in Example 1 to reveal free acid, resuming with the steps in Example 13 | |
| 20 | 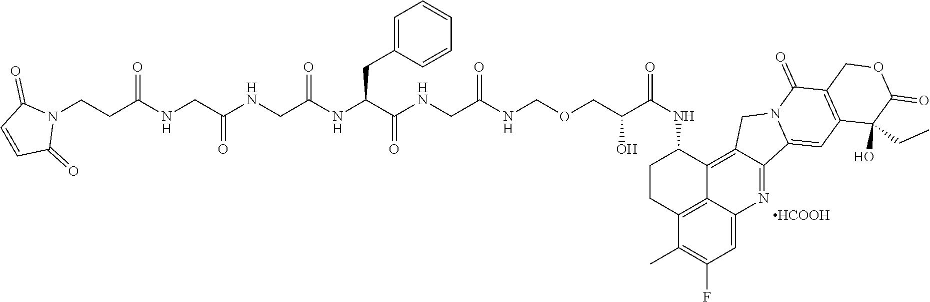 | 1044 [M + Na] |
| | From I-1, from methyl (R)-2,3-dihydroxypropanoate instead of 13a, then using the conditions of step C in Example 1 to reveal free acid, resuming | |
| | with the steps in Example 13 | |
| 21 | 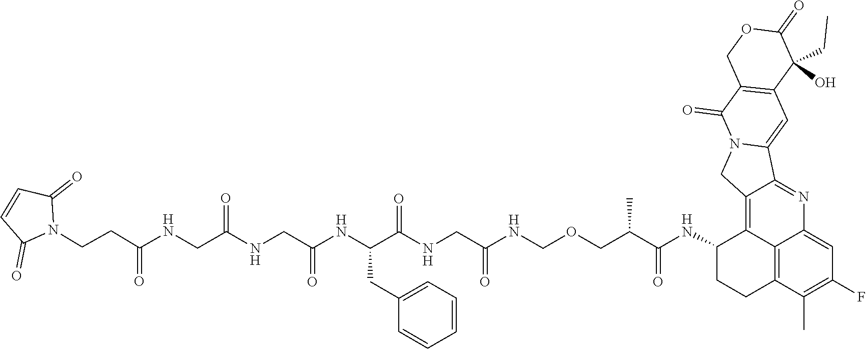 [SMILES: CC[C@@]1(O)C(=O)OCc2c1cc1n(c2=O)Cc2c-1nc1cc(F)c(C)c3c1c2[C@@H](NC(=O)[C@@H](C)COCNC(=O)CNC(=O)[C@H](Cc1ccccc1)NC(=O)CNC(=O)CNC(=O)CCN1C(=O)C=CC1=O)CC3] | 1042 [M + Na] |
| | From I-1 and (S)-3-hydroxy-2-methylpropanoic acid instead of 13a | |
| 22 | 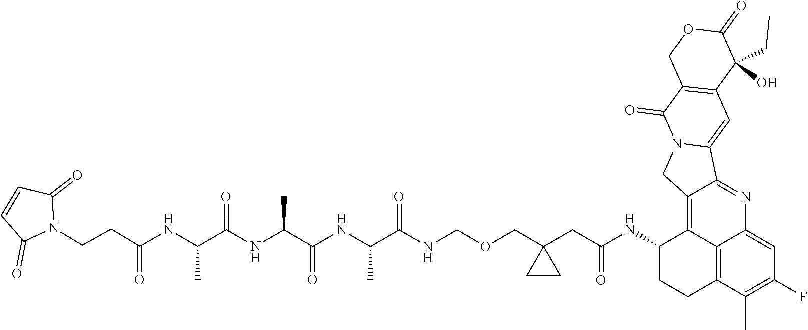 [SMILES: CC[C@@]1(O)C(=O)OCc2c1cc1n(c2=O)Cc2c-1nc1cc(F)c(C)c3c1c2[C@@H](NC(=O)CC1(COCNC(=O)[C@H](C)NC(=O)[C@H](C)NC(=O)[C@H](C)NC(=O)CCN2C(=O)C=CC2=O)CC1)CC3] | 941 |
| | From I-8 and 2-(1-(hydroxymethyl)cyclopropyl)acetic acid instead of 13a | |
| 23 (peak 1) | 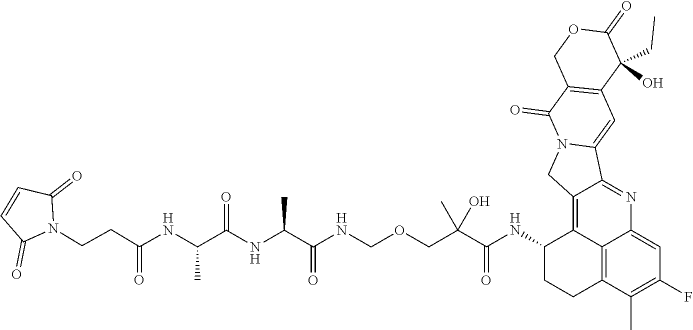 [SMILES: CC[C@@]1(O)C(=O)OCc2c1cc1n(c2=O)Cc2c-1nc1cc(F)c(C)c3c1c2[C@@H](NC(=O)C(C)(O)COCNC(=O)[C@H](C)NC(=O)[C@H](C)NC(=O)CCN1C(=O)C=CC1=O)CC3] | 860 |
| | From 2,3-dihydroxy-2-methylpropanoic acid instead of 13a | |
| 24 (peak 2) | 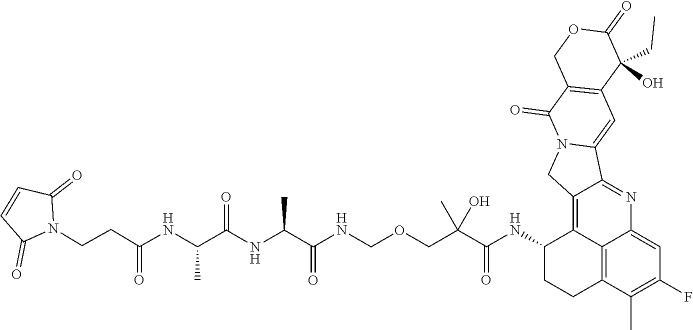 [SMILES: CC[C@@]1(O)C(=O)OCc2c1cc1n(c2=O)Cc2c-1nc1cc(F)c(C)c3c1c2[C@@H](NC(=O)C(C)(O)COCNC(=O)[C@H](C)NC(=O)[C@H](C)NC(=O)CCN1C(=O)C=CC1=O)CC3] | 860 |
| | From 2,3-dihydroxy-2-methylpropanoic acid instead of 13a | |
| 25 | 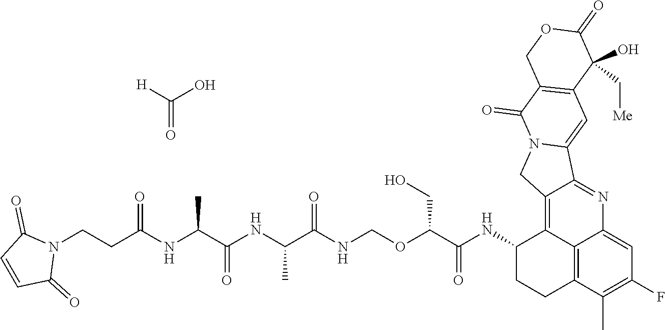 [SMILES: CC[C@@]1(O)C(=O)OCc2c1cc1n(c2=O)Cc2c-1nc1cc(F)c(C)c3c1c2[C@@H](NC(=O)[C@@H](CO)OCNC(=O)[C@H](C)NC(=O)[C@H](C)NC(=O)CCN1C(=O)C=CC1=O)CC3 ; O=CO] | 846 |
| | From I-27, using only step C in Example 13 above without the first 4-methylpiperidine phase, and | |
| | instead using DIPEA as base at RT | |

### Example 26

### Preparation of Example 26

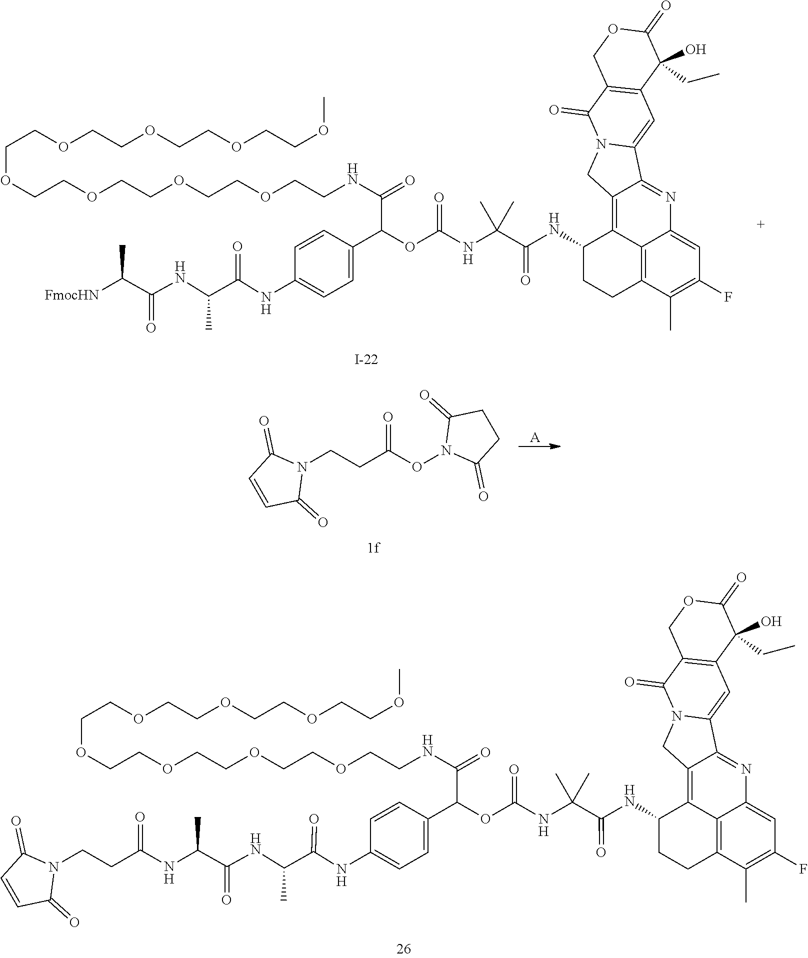

### Step A—Synthesis of Compound 26

A mixture of 28-(4-((S)-2-((S)-2-((((9H-fluoren-9-yl)methoxy)carbonyl)amino)propanamido)propanamido)phenyl)-27-oxo-2,5,8,11,14,17,20,23-octaoxa-26-azaoctacosan-28-yl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (I-22, 9.1 mg, 0.0063 mmol), 4-methylpiperidine (1.3 μL, 0.011 mmol) and DMF (0.60 mL) was stirred at RT for 6 h. To the reaction mixture was then added 2,5-dioxopyrrolidin-1-yl 3-(2,5-dioxo-2,5-dihydro-1H-pyrrol-1-yl)propanoate (1f, 6.0 mg, 0.023 mmol). After stirring at RT for 15 minutes, additional 2,5-dioxopyrrolidin-1-yl 3-(2,5-dioxo-2,5-dihydro-1H-pyrrol-1-yl)propanoate (6.0 mg, 0.023 mmol) was added. The reaction mixture was stirred at RT for an additional 10 minutes and then directly purified via reverse phase column chromatography (10-70% MeCN/water with 0.1% formic acid modifier) to afford 28-(4-((S)-2-((S)-2-(3-(2,5-dioxo-2,5-dihydro-1H-pyrrol-1-yl)propanamido)propanamido)propanamido)phenyl)-27-oxo-2,5,8,11,14,17,20,23-octaoxa-26-azaoctacosan-28-yl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1l-yl)amino)-2-methyl-1-oxopropan-2-yl)carbamate (26). MS: m/z=1395 [M+Na]. ¹H NMR (500 MHz, DMSO-d₆) δ 9.83 (s, 1H), 8.22-8.15 (m, 2H), 8.12-8.06 (m, 1H), 7.79 (d, J=11.3 Hz, 1H), 7.57-7.45 (m, 3H), 7.35-7.18 (m, 3H), 6.99 (s, 2H), 6.53-6.49 (m, 4H), 5.61 (d, J=4.1 Hz, 1H), 5.52-5.36 (m, 3H), 5.25-5.13 (m, 2H), 4.40-4.31 (m, 1H), 4.26-4.17 (m, 1H), 3.60 (t, J=7.2 Hz, 2H), 3.51-3.40 (m, 26H), 3.22 (d, J=1.7 Hz, 3H), 3.16-3.07 (m, 2H), 2.39 (t, J=6.5 Hz, 2H), 2.18-1.93 (m, 2H), 1.93-1.79 (m, 2H), 1.44-1.13 (m, 12H), 0.87 (t, J=7.4 Hz, 3H).

The following compounds of the present disclosure in Table 8 were made using similar methods described in Example 26, with subtle variations in reaction times and substituting the appropriate reactants and/or reagents (1-22, and/or 1f (with other appropriate commercially

TABLE 8

| | | MS |
|---|---|---|
| Ex. # | Structure | [M + Na] |
| 27 | 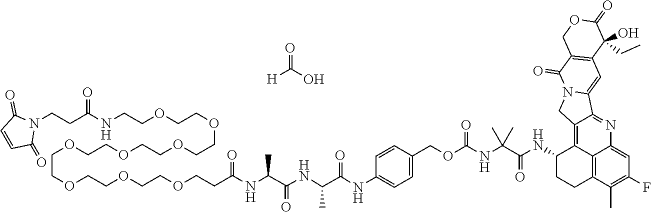 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)CCOCCOCCOCCOCCOCCOCCOCCOCCNC(=O)CCN2C(=O)C=CC2=O)cc1)CC3 ; O=CO] | 1409 |
| | from I-21, with 4-methylpiperidine and DIPEA | |
| I-29 | 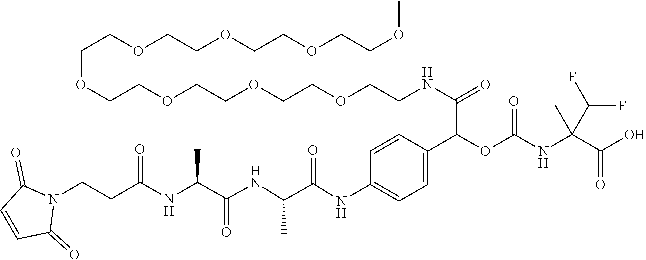 [SMILES: COCCOCCOCCOCCOCCOCCOCCOCCNC(=O)C(OC(=O)NC(C)(C(=O)O)C(F)F)c1ccc(NC(=O)[C@H](C)NC(=O)[C@H](C)NC(=O)CCN2C(=O)C=CC2=O)cc1] | 1013 |
| | from I-24, with 4-methylpiperidine and NMM; 23-40° C. during Fmoc deprotection | |
| I-30 | 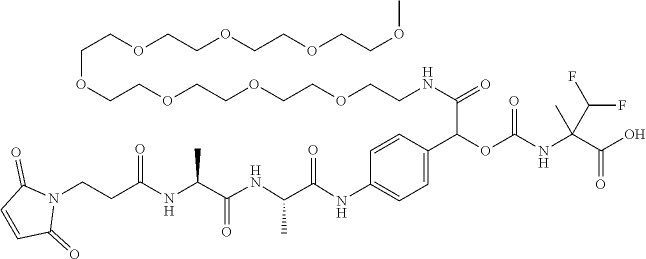 [SMILES: COCCOCCOCCOCCOCCOCCOCCOCCNC(=O)C(OC(=O)NC(C)(C(=O)O)C(F)F)c1ccc(NC(=O)[C@H](C)NC(=O)[C@H](C)NC(=O)CCN2C(=O)C=CC2=O)cc1] | 1013 |
| | from I-25, with 4-methylpiperidine and NMM; 40° C. during Fmoc-deprotection | |

### Example 28

### Preparation of Example 28

### Step A—Synthesis of Compound 28a

A mixture of 1-(4-((S)-2-((S)-2-((tert-butoxycarbonyl)amino)propanamido)propanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12H-

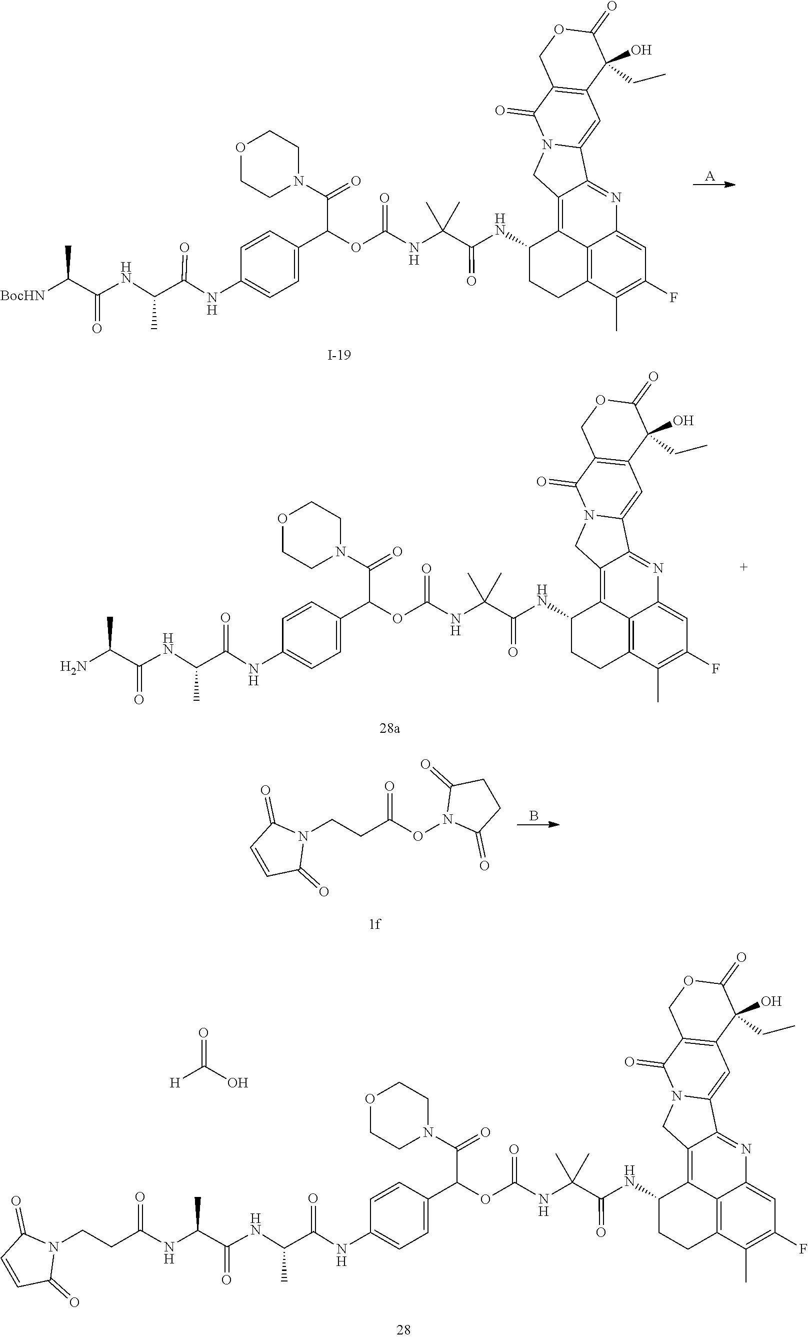

benzo[de]pyrano[3′,4′:6,7]indolizino[1,2-b]quinolin-1-yl)amino)-2-methyl-1-oxopropan-2-yl)carbamate (1-19, 18.5 mg, 0.018 mmol), TFA (28 μL, 0.36 mmol) and 1,2-dichloroethane (0.50 mL) was stirred at RT for 3 h The reaction mixture was then diluted with DMSO and purified by reverse phase column chromatography (10-50% MeCN/water with 0.1% formic acid modifier) to afford 1-(4-((S)-2-((S)-2-aminopropanamido)propanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (28a). MS: m/z=925 [M+H].

### Step B—Synthesis of Compound 28

A mixture of 1-(4-((S)-2-((S)-2-aminopropanamido)propanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (28a, 5.9 mg, 0.0061 mmol), DIPEA (2.1 μL, 0.012 mmol), 2,5-dioxopyrrolidin-1-yl 3-(2,5-dioxo-2,5-dihydro-1H-pyrrol-1-yl)propanoate (1f, 4.9 mg, 0.018 mmol) and DMF (0.50 mL) was stirred at RT for 20 min. The reaction mixture was then directly purified by reverse phase column chromatography (10-60% MeCN/water with 0.1% formic acid modifier) to afford 1-(4-((S)-2-((S)-2-(3-(2,5-dioxo-2,5-dihydro-1H-pyrrol-1-yl)propanamido)propanamido)propanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate formate (28). MS: m/z=1076 [M+H]. ¹H NMR (500 MHz, DMSO-d₆) δ 9.88 (d, J=10.5 Hz, 1H), 8.53 (s, 1H), 8.23-8.18 (m, 1H), 8.13 (d, J=7.1 Hz, 1H), 8.08-8.00 (m, 1H), 7.79 (d, J=11.1 Hz, 1H), 7.66-7.48 (m, 3H), 7.39-7.28 (m, 2H), 7.02-6.94 (m, 3H), 6.76 (s, 1H), 6.58-6.51 (m, 1H), 6.12 (s, 0.5H), 6.03 (s, 0.5H), 5.54-5.12 (m, 5H), 4.40-4.30 (m, 1H), 4.25-4.17 (m, 1H), 3.60 (t, J=7.2 Hz, 2H), 3.53-3.39 (m, 3H), 3.19-3.03 (s, 4H), 2.99-2.82 (m, 1H), 2.43-2.37 (m, 5H), 2.23-2.12 (m, 1H), 2.10-1.97 (m, 1H), 1.93-1.76 (m, 2H), 1.48-1.25 (m, 9H), 1.16 (d, J=7.1 Hz, 3H), 0.91-0.80 (m, 3H).

The following compounds of the present disclosure in Table 9 were made using similar methods described in Example 28, with subtle variations in reaction times and substituting the appropriate reactants and/or reagents (I-19, and/or 1f (with other appropriate commercially available NHS esters)):

TABLE 9

| | | MS |
|---|---|---|
| Ex. # | Structure | [M + H] |
| 29 | 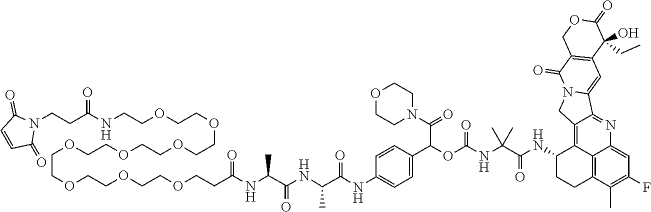 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OC(C(=O)N1CCOCC1)c1ccc(NC(=O)[C@H](C)NC(=O)[C@H](C)NC(=O)CCOCCOCCOCCOCCOCCOCCOCCOCCNC(=O)CCN2C(=O)C=CC2=O)cc1)CC3] | 1499.6 |
| 30 | 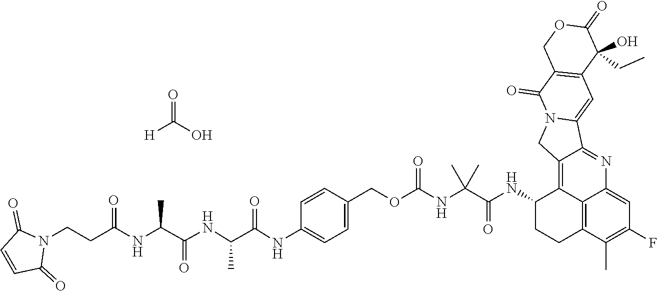 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)CCN2C(=O)C=CC2=O)cc1)CC3 ; O=CO] | 963 |
| | from I-20 | |
| 31 | 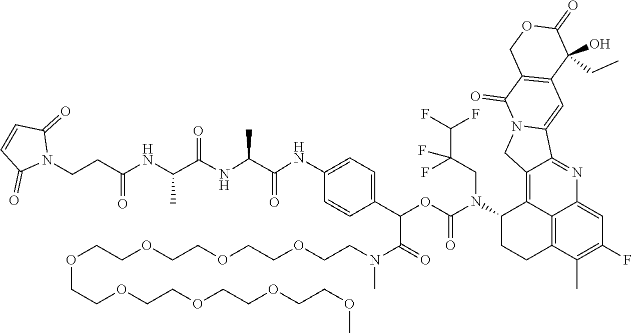 | m/2 719 [M + H + Na] |
| | from I-23 | |
| I-31 | 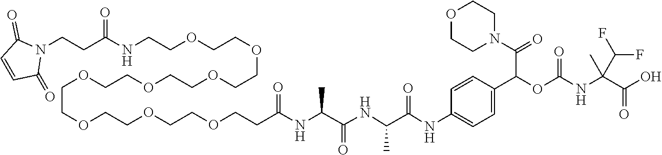 [SMILES: C[C@H](NC(=O)CCOCCOCCOCCOCCOCCOCCOCCOCCNC(=O)CCN1C(=O)C=CC1=O)C(=O)N[C@@H](C)C(=O)Nc1ccc(C(OC(=O)NC(C)(C(=O)O)C(F)F)C(=O)N2CCOCC2)cc1] | 1140 [M + Na] |
| | from I-26, with no intermediate purification at Step A | |

### Example 32

### Preparation of Example 32

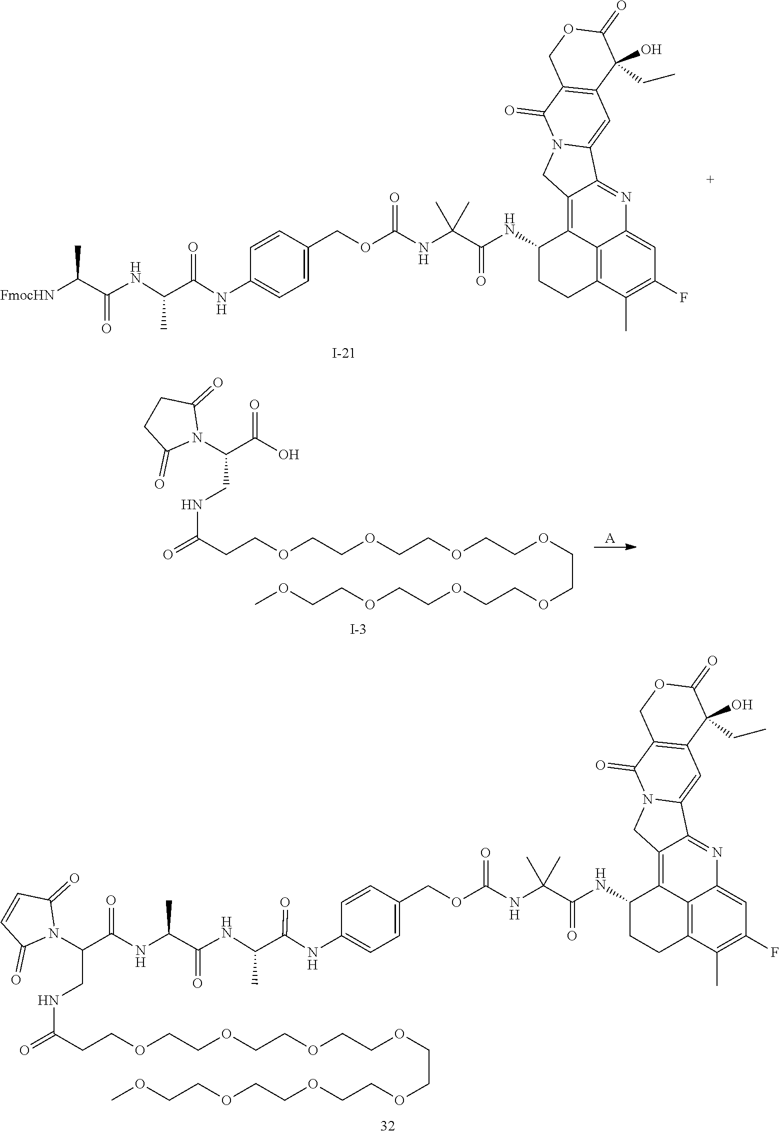

### Step A—Synthesis of Compound 32

Mixture A: A mixture of (9H-fluoren-9-yl)methyl ((S)-1-(((S)-1-((4-((((1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamoyl)oxy)methyl)phenyl)amino)-1-oxopropan-2-yl)amino)-1-oxopropan-2-yl)carbamate (I-21, 31 mg, 0.030 mmol) and NMM (13 μL, 0.12 mmol) in DMF (0.30 mL) was stirred at 50° C. overnight. Afterwards, it was cooled to 0° C.

Mixture B: In a separate vial, a mixture of (S)-29-(2,5-dioxo-2,5-dihydro-1H-pyrrol-1-yl)-26-oxo-2,5,8,11,14,17,20,23-octaoxa-27-azatriacontan-30-oic acid (I-3, 47 mg, 0.082 mmol) and COMU (23 mg, 0.054 mmol) in DMF (0.30 mL) was cooled to 0° C., at which point 2,6-lutidine (19 μL, 0.17 mmol) was added. The mixture was stirred at 0° C. for 30 min, and then added to pre-cooled Mixture A.

The combined reaction mixture was stirred at 0° C. for 40 min, and then directly purified by reverse phase column chromatography (10-60% MeCN/water with 0.1% formic acid modifier) to afford 4-((32S,35S)-29-(2,5-dioxo-2,5-dihydro-1H-pyrrol-1-yl)-32,35-dimethyl-26,30,33-trioxo-2,5,8,11,14,17,20,23-octaoxa-27,31,34-triazahexatriacontan-36-amido)benzyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-methyl-1-oxopropan-2-yl)carbamate (32). Assumed 4:1 dr at maleimide stereocenter by NMR. MS: m/z=687 [M+2H]. ¹H NMR (500 MHz, DMSO-d₆) δ 9.86 (s, 1H), 8.30 (d, J=7.1 Hz, 1H), 8.17 (d, J=7.3 Hz, 1H), 8.12 (d, J=7.1 Hz, 1H), 7.90 (t, J=6.2 Hz, 1H), 7.79 (d, J=11.1 Hz, 1H), 7.50 (d, J=8.0 Hz, 2H), 7.31 (s, 1H), 7.20 (s, 1H), 7.11-7.05 (m, 2H), 7.02 (s, 2H), 6.72 (br s, 2H), 6.50 (s, 1H), 5.52-5.45 (m, 1H), 5.45-5.34 (m, 2H), 5.31-5.17 (m, 2H), 4.86-4.74 (m, 2H), 4.58 (dd, J=10.2, 4.4 Hz, 1H), 4.42-4.34 (m, 1H), 4.31-4.23 (m, 1H), 3.82-3.73 (m, 1H), 3.55-3.40 (m, 30H), 3.23 (s, 3H), 3.12-3.02 (m, 2H), 2.38 (s, 3H), 2.24-2.07 (m, 3H), 2.07-1.96 (m, 1H), 1.89-1.76 (m, 2H), 1.38 (s, 6H), 1.31 (d, J=7.0 Hz, 3H), 1.14 (d, J=7.2 Hz, 3H), 0.81 (t, J=7.2 Hz, 3H).

### Example 33

### Preparation of Example 33

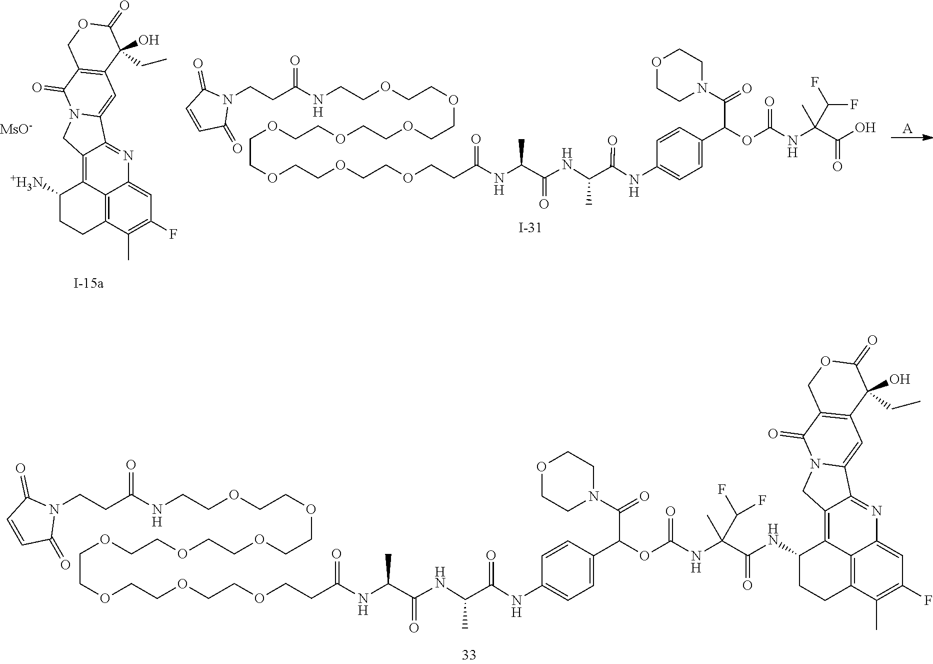

### Step A—Synthesis of Compound 33

A mixture of 2-(((1-(4-((2S,5S)-37-(2,5-dioxo-2,5-dihydro-1H-pyrrol-1-yl)-2,5-dimethyl-4,7,35-trioxo-10,13,16,19,22,25,28,31-octaoxa-3,6,34-triazaheptatriacontanamido)phenyl)-2-morpholino-2-oxoethoxy)carbonyl)amino)-3,3-difluoro-2-methylpropanoic acid (1-31, 16 mg, 0.014 mmol), NMM (1.9 μL, 0.017 mmol) and HATU (6.5 mg, 0.017 mmol) in DMF (0.50 mL) was stirred at RT for 2 min, at which point was added (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (I-15a, 46 μL, 0.016-0.018 mmol, ˜0.35-0.40 M in DMF, with 3 eq. TEA). The reaction mixture was stirred at RT for 10 min, then directly purified by reverse phase column chromatography (10-70% MeCN/water with 0.1% formic acid modifier) to afford 1-(4-((2S,5S)-37-(2,5-dioxo-2,5-dihydro-1H-pyrrol-1-yl)-2,5-dimethyl-4,7,35-trioxo-10,13,16,19,22,25,28,31-octaoxa-3,6,34-triazaheptatriacontanamido)phenyl)-2-morpholino-2-oxoethyl (1-(((1S,9S)-9-ethyl-5-fluoro-9-hydroxy-4-methyl-10,13-dioxo-2,3,9,10,13,15-hexahydro-1H,12Hbenzo[de]pyrano[3′,4′:6,7]indolizino[1,2-b]quinolin-1-yl)amino)-3,3-difluoro-2-methyl-1-oxopropan-2-yl)carbamate (33). MS: m/2=768.6 [M+2H]. ¹H NMR (500 MHz, DMSO-d₆) δ 9.96 (d, J=17.3 Hz, 1H), 8.58-8.46 (m, 1H), 8.33 (s, 1H), 8.22-7.98 (m, 4H), 7.80 (t, J=9.9 Hz, 1H), 7.67 (d, J=8.6 Hz, 1H), 7.60 (d, J=8.2 Hz, 1H), 7.39 (d, J=8.7 Hz, 1H), 7.37-7.24 (m, 2H), 7.00 (s, 2H), 6.53 (s, 1H), 6.51-6.16 (m, 2H), 5.56-5.32 (m, 3H), 5.28-5.07 (m, 2H), 4.42-4.33 (m, 1H), 4.32-4.24 (m, 1H), 3.59 (t, J=7.2 Hz, 4H), 3.50 (d, J=4.1 Hz, 30H), 3.18-3.12 (m, 2H), 2.45-2.36 (m, 5H), 2.33 (t, J=7.3 Hz, 2H), 2.22-1.96 (m, 2H), 1.94-1.79 (m, 2H), 1.42 (d, J=14.0 Hz, 3H), 1.30 (t, J=7.5 Hz, 3H), 1.23-1.17 (m, 3H), 0.91-0.84 (in, 3H).

The following compounds of the present disclosure in Table 10 were made using similar methods described in Example 33, with subtle variation in reaction times and substituting the appropriate reactants and/or reagents (I-31):

TABLE 10

| | | MS |
|---|---|---|
| Example | Structure | [M + H] |
| 34 | 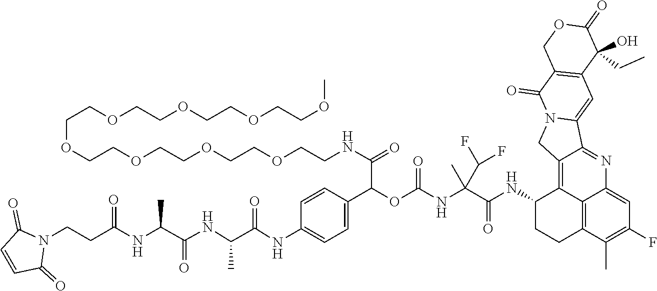 | m/2 716 [M +H + Na] |
| | From 1-29 (1-15a pre-neutralized with NMM instead of TEA) | |
| 35 | 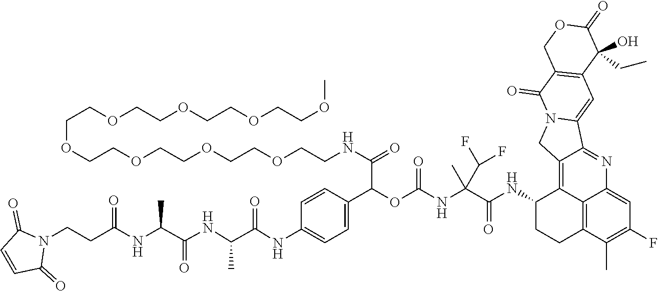 | m/2 716 [M + H + Na] |
| | From I-30 (1-15a pre-neutralized with NMM instead of TEA) | |

### Standard Conjugation Protocol

Illustrative compounds of the present disclosure were conjugated to either an anti-ROR1 antibody (Cirmtuzumab) and/or an anti-Trop2 antibody (Sacituzumab) utilizing the following conjugation protocol:

### Example 36

The antibody (Cirmtuzumab, 20 mg) was exchanged into PBS (10 mM pH 7.4) and taken to a concentration of 10 mg/mL in PBS (1500 μL) and Tris/EDTA (400 μL) (10% v/v 500 mM pH 8 TRIS/90% 25 mM EDTA). An aqueous solution of 3,3′,3″-phosphanetriyltripropionic acid hydrochloride (6 eq., 81 μL, 0.810 μmol) was added, and the mAb was reduced for 2 h at rt. DMSO (100 μL) was added, followed by a 10 mM solution of 1 (135 uL, 10 eq.) in DMSO. The reaction was then mixed at RT overnight.

The mixture was then purified and exchanged into pH 5.5 20 mM sodium acetate buffer with a desalting column (AKTA™ chromatography system, monitoring at 280 nm), followed by the addition of 9% sucrose, to afford ADC example 36. The solution was characterized by LCMS (Agilent PLRP-S column, 1000 Å, 5 μm, 15-90% MeCN/H₂O with 0.1% formic acid, 80° C. column temperature) and SEC (Acquity UPLC Protein BEH SEC, 200 Å, 1.7 μm, 100 mM sodium phosphate, 200 mM NaCl, 0.02% azide, 5% IPA added to mobile phase for hydrophobic ADCs).

### ROR1 ADC Data

The following Table 11 demonstrates average DAR and percent aggregation for ROR1 ADCs utilizing the illustrative compounds of the present disclosure and the aforementioned conjugation protocol:

TABLE 11

| | Linker- | | |
|---|---|---|---|
| ADC | Payload | Avg. | |
| Example | Example | DAR | % Aggregation* |
| 36 | 1 | 8 | 3.8 |
| 37 | 2 | 7.2 | 6.1 |
| 38 | 3 | 7.1 | 4.6 |
| 39 | 4 | 7 | 4.9 |
| 40 | 5 | 7.9 | 3.7 |
| 41 | 6 | 8 | 2.9 |
| 42 | 7 | 8 | 2.4 |
| 43 | 12 | 7.9 | 2.6 |
| 44 | 13 | 8 | 4.0 |
| 45 | 15 | 6.9 | 5.0 |
| 46 | 16 | 7.7 | 3.0 |
| 47 | 17 | 7.5 | 3.4 |
| 48 | 18 | 7.7 | 4.8 |
| 49 | 19 | 8 | 2.4 |
| 50 | 20 | 7.1 | 9.0 |
| 51 | 21 | 8 | 2.8 |
| 52 | 22 | 6.7 | 6.2 |
| 53 | 23 | 8 | 2.6 |
| 54 | 24 | 7.8 | 3.6 |
| 55 | 25 | 8 | 2.5 |
| 56 | 26 | 7.8 | 2.9 |
| 57 | 27 | 7.9 | 2.5 |
| 58 | 28 | 8 | 10.1 |
| 59 | 29 | 8 | 6.7 |
| 60 | 30 | 7.9 | 4.3 |
| 61 | 33 | 7.8 | 4.6 |
| 62 | 34 | 8 | 2.4 |
| 63 | 35 | 7.8 | 2.5 |

*Note:
Baseline parental Cirmtuzumab aggregation~2%

### ROR1 EMT6 Cytotoxicity Assay Protocol

Illustrative ROR1 ADCs of the present disclosure were subjected to a cell-based cytotoxicity assay (EMT6 cells) utilizing the following protocol:

EMT6 cells were washed once with PBS (without calcium or magnesium), then 5 mL of 0.25% Trypsin-EDTA (catalog number is 25200056 from Thermo Fisher) was added, and the flask was incubated at 37° C. for ˜3 minutes. Then, 10 mL of cell culture medium (RPMI 1640 (Gibco™ 72400-047)+10% FBS (Gibco™ 26140-079)) was added, with pipetting up and down a few times to dissociate cells. The cells were transferred to a 15 mL conical tube, and centrifuged at 300 g for 5 minutes. The cell pellet was then resuspended in 2 mL of cell culture medium, and cells were counted with a Vi-CELL (viability of all cell lines was >95%). The cells were then plated onto 96-well plates (Corning™ 3904), with 200 viable cells in 90 μL of cell culture medium per well and incubated in a cell culture incubator overnight.

The next day, serial dilutions of the ADCs were prepared in cell culture medium (starting concentration for ADCs was 1 μM, dilution factor is 1/5), and 10 μL of dilutes was added into each well (total volume is 100 μL per well). Only the inner 60 wells on the plate were used for drug treatment and no-treatment controls. The plate was then incubated in a cell culture incubator for another 96 hours.

After the 4-day treatment, the plate and its contents were equilibrated at RT for approximately 30 minutes. Additionally, the CellTiter-Glo™ Buffer was thawed and equilibrated to RT, and the appropriate volume of CellTiter-Glo™ Buffer was transferred into the amber bottle containing CellTiter-Glo™ Substrate to reconstitute the lyophilized enzyme/substrate mixture (Promega™ catalog #G7573). Then, 100 μL of CellTiter-Glo™ Reagent was added to each well, and the contents were mixed for 2 minutes on an orbital shaker to induce cell lysis. The plate was allowed to incubate at RT for 10 minutes to stabilize luminescent signal. The luminescence was recorded on a PerkinElmer Multimode Plate Reader EnVision™. The data was analyzed with GraphPad Prism 8 to generate an EC₅₀.

Illustrative ROR1 ADCs of the present disclosure were tested in the above EMT6 cytotoxicity assay, and results are provided in Table 12 below:

TABLE 12

| ADC Example | EC₅₀ (nM) |
|---|---|
| 36 | 1.32 |
| 37 | 4.89 |
| 38 | 2.85 |
| 39 | 2.4 |
| 40 | 1.42 |
| 41 | 6.16 |
| 42 | 1.96 |
| 43 | 23.36 |
| 44 | 22.44 |
| 45 | 0.7 |
| 46 | 110.9 |
| 47 | 31 |
| 48 | 1.3 |
| 49 | 2.88 |
| 50 | 0.28 |
| 51 | 27.53 |
| 52 | 33.86 |
| 53 | 0.33 |
| 54 | 0.65 |
| 55 | 0.46 |
| 56 | 8.55 |
| 57 | 5.27 |
| 58 | 86.32 |
| 59 | 64.16 |
| 60 | 13.82 |
| 61 | 3.25 |
| 62 | 9.9 |
| 63 | 3.81 |

### Example 64

The antibody (Sacituzumab, 20 mg) was exchanged into PBS (10 mM pH 7.4) and taken to a concentration of 10 mg/mL in PBS (1500 μL) and Tris/EDTA (400 μL) (10% v/v 500 mM pH 8 TRIS/90% 25 mM EDTA). An aqueous solution of 3,3′,3″-phosphanetriyltripropionic acid hydrochloride (6 eq., 81 μL, 0.810 μmol) was added, and the mAb was reduced for 2 h at rt. DMSO (100 μL) was added, followed by a 10 mM solution of 1 (135 uL, 10 eq.) in DMSO. The reaction was then mixed at RT overnight. If the reaction was deemed incomplete, up to 4 additional equivalents of linker-payload were added.

The mixture was then purified and exchanged into either pH 5.5 20 mM sodium acetate buffer or pH 6.5 10 mM histidine buffer with a desalting column (AKTA™ chromatography system, monitoring at 280 nm), followed by the addition of 9% sucrose, to afford ADC example 64. The solution was characterized by LCMS (Agilent PLRP-S column, 1000 Å, 5 μm, 15-90% MeCN/H₂O with 0.1% formic acid, 80° C. column temperature) and SEC (Acquity UPLC Protein BEH SEC, 200 Å, 1.7 μm, 100 mM sodium phosphate, 200 mM NaCl, 0.02% azide, 5% IPA added to mobile phase for hydrophobic ADCs).

### Trop2 ADC Data

The following Table 13 demonstrates average DAR and percent aggregation for Trop2 ADCs utilizing the illustrative compounds of the present disclosure and the aforementioned conjugation protocol:

TABLE 13

| | Linker- | | |
|---|---|---|---|
| ADC | Payload | Avg. | |
| Example | Example | DAR | % Aggregation |
| 64 | 1 | 8 | 0.6 |
| 65 | 2 | 7.8 | 2.1 |
| 66 | 5 | 8 | 0.9 |
| 67 | 6 | 8 | 0.7 |
| 68 | 7 | 8 | 1.2 |
| 69 | 8 | 7 | 3.7 |
| 70 | 15 | 8 | 0.4 |
| 71 | 20 | 7.8 | 3.5 |
| 72 | 23 | 8 | 0.6 |
| 73 | 26 | 8.4 | 0.0 |
| 74 | 27 | 7.5 | 1.6 |
| 75 | 31 | 1.6* | 0.0 |
| 76 | 32 | 7.8 | 1.8 |

*Note:
Conjugation procedure for DAR2 is as follows:
Sacituzumab with two engineered Cys residues was decapped using a literature procedure (WO2017072662) and diluted to 5 mg/mL with PBS. Then 9% DMSO was added and 28-(4-((S)-2-((S)-2-(3-(2,5-dioxo-2,5-dihydro-1H-pyrrol-1-yl)propanamido)propanamido)propanamido)phenyl)-26-methyl-27-oxo-2,5,8,11,14,17,20,23-octaoxa-26-azaoctacosan-28-yl ((1S,9S)-9-ethyl-5-fluoro-9-hydroxy-4-methyl-10,13-dioxo-2,3,9,10,13,15-hexahydro-1H,12H- benzo[de]pyrano[3′,4′:6,7]indolizino[1,2-b]quinolin-1-yl)(2,2,3,3-tetrafluoropropyl)carbamate (31, 27 μl, 0.27 μmol) was added as a 10 mM DMSO solution, and the reaction was mixed at RT for 2 hr. The ADC was purified via AKTA ™ (desalting column, histidine pH 6.5 buffer, monitoring at 280 nm) and characterized via LCMS and SEC (as described above).

### Trop2 N87 Cytotoxicity Assay Protocol

Illustrative Trop2 ADCs of the present disclosure were subjected to a cell-based cytotoxicity assay (NCI-N87 cells) utilizing the following protocol:

Step 1: Seed 384-well Plates for Assay (45 uL per well) on day 0
Cells (NCI-N87) were quickly thawed in a cryo-vial by incubating them in a 37° C. water bath for <1 min until there is just a small bit of ice left in the vial. The vial was promptly removed and wiped down with 70% ethanol. The cells were transferred from the vial to a sterile centrifuge tube containing 8 mL of pre-warmed cell culture medium (RPMI-1640 (Cat #30-2001)+10% FBS+1% P/S). The vial was flushed with an additional 1 mL of medium to ensure complete transfer of cells to the centrifuge tube. The cells were then centrifuged at 150 g for 5 minutes. The supernatant was aspirated, and the cell pellet was resuspended in 10-20 mL cell culture medium (RPMI-1640 (Cat #30-2001)+10% FBS+1% P/S). Cells were counted using Vi-cell and prepared 1,500 cells/45 uL per well. Then added 45 uL/well of cells into Corning® 384-well Low Flange White Flat Bottom Polystyrene TC-treated Microplates (Corning, Cat #3570) using Standard Cassette Combi (if needed, dispense 1 dummy plate at 20 uL to help normalize Combi, using medium speed). The plates were spun down at 150 g for 30 seconds.

### Step 2: Add ADCs on day 1

The ADC vials and reference stock were taken out and allowed to thaw at RT. The tubes were centrifuged at 2000 g for 30 seconds. The 10× Intermediate assay plates (Waters plate, Cat #186002632) were prepared using a Bravo liquid handler. Use proper buffer (10 mM pH 6.5 histidine 9% sucrose buffer) to make serial dilutions. Media (no cells) was used for Max_E. Then added 5 uL of 10× stock from intermediate plate to assay plate using a Bravo liquid handler using a very slow speed so the cell monolayer wasn't disturbed. The plates were spun down at 150 g for 30 seconds.
Step 3: CellTiter-Glo 2.0 Assay (Promega, Cat #G9242) on day 7 (CellTiter-Glo kit stored at −70° C.)
The CellTiter-Glo® 2.0 Reagent was thawed at 4° C. overnight (Did not expose the reagent to temperatures above 25° C.). The kit was equilibrated to RT for approximately 30 minutes. Added 20 uL of CellTiter-Glo® 2.0 Reagent to 50 uL of medium containing cells using Standard Cassette Combi. The contents were mixed for 2-3 minutes on an orbital shaker to induce cell lysis. The plates were spun down at 150 g for 30 seconds. The plates were allowed to incubate at RT for 5 minutes to stabilize the luminescent signal. The luminescence was recorded to calculate an EC₅₀ value, using an integration time of 0.25-1 second per well as a guideline.

Illustrative Trop2 ADCs of the present disclosure were tested in the above NCI-N87 cytotoxicity assay, and results are provided in Table 14 below:

TABLE 14

| ADC Example | EC₅₀ (nM) |
|---|---|
| 64 | 0.51 |
| 65 | 0.85 |
| 66 | 0.20 |
| 67 | 0.070* |
| 68 | 1.03 |
| 69 | 2.11 |
| 70 | 0.80 |
| 71 | 1.88 |
| 72 | 4.73 |
| 73 | 5.66 |
| 74 | 3.73 |
| 75 | 6.26 |
| 76 | 25.30 |

*Note:
Cytotoxicity assay was performed on a Trop2-expressing EMT6 cell line, with RPMI 1640 (Gibco ™ 72400-047) + 10% FBS (Gibco ™ 26140-079) culture medium, and 4-day treatment.

What is claimed:

1. A compound having a structural Formula I, or salt thereof: 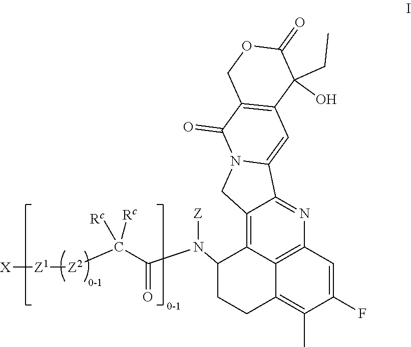
wherein:
Z is selected from hydrogen and —CH₂C(R^{x})(R^{y})CHF₂;
Z¹ is selected from —NH— and —O—;
Z² is absent or selected from —CR^{b}R^{b}—, —CH₂CR^{b}R^{b}—, and —CR^{b}R^{b}CH₂—;
each R^{b} is independently selected from hydrogen, —C₁₋₆ alkyl, and hydroxyl;
or two adjacent R^{b} combine to form spirocycloalkyl;
each R^{c} is independently selected from hydrogen, —C₁₋₆ alkyl, halogen, and hydroxyl;
or two adjacent R^{c} combine to form spirocycloalkyl;
R^{x} and R^{y} are independently selected from hydrogen, C₁₋₆ alkyl, C₁₋₃ haloalkyl, halogen, hydroxyl, and —C₁₋₆ alkyl-OH;
or R^{x} and R^{y} combine to form a C₃₋₆cycloalkyl, or spirocycloalkyl;
X is a linking group selected from: 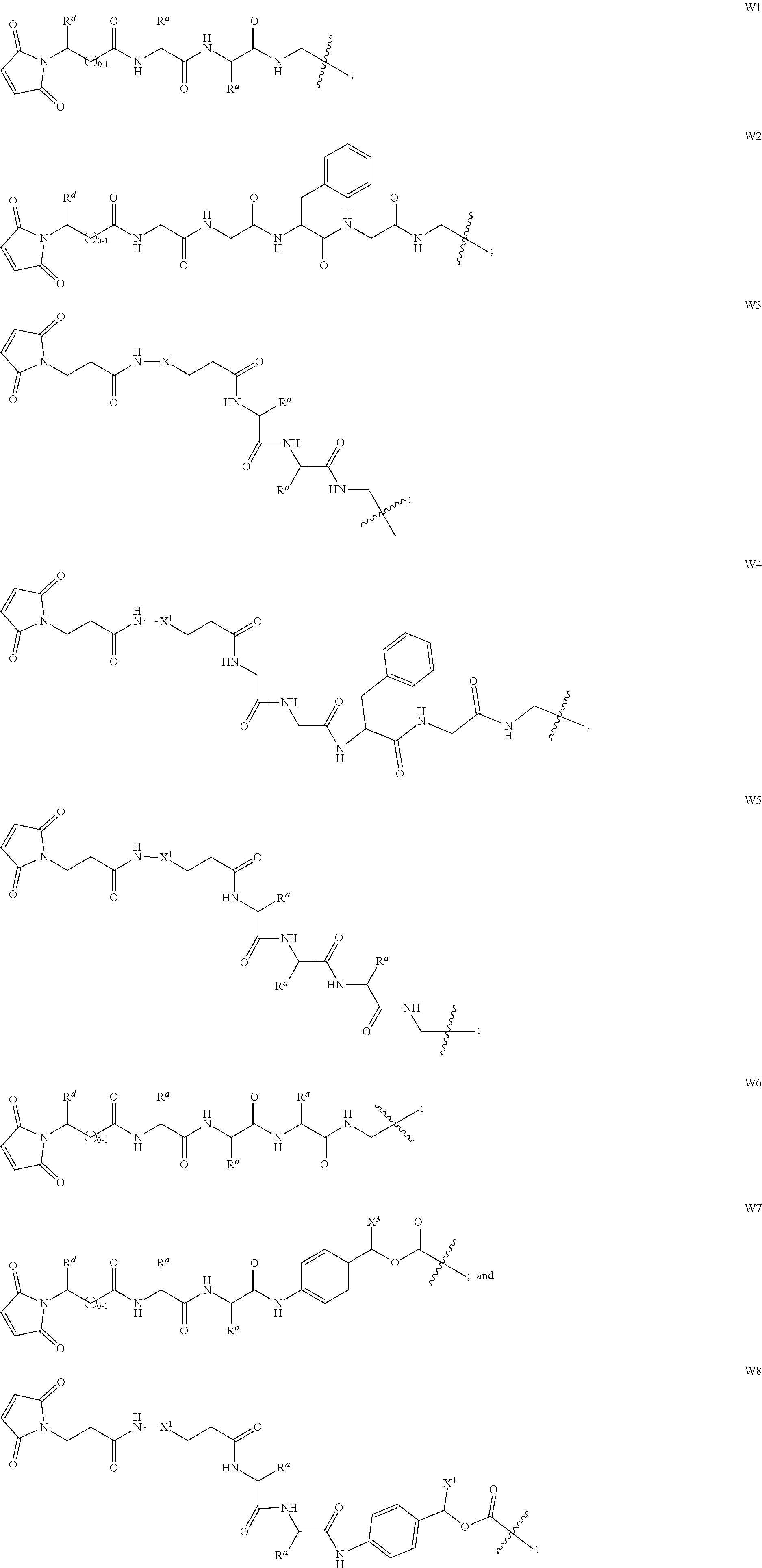 [symbol]
represents point of attachment to structural Formula I;
X¹ is a polyethylene glycol (PEG);
X³ is hydrogen or —C(O)NR^{a}R^{z},
X⁴ is hydrogen or 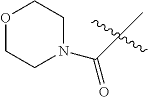 [SMILES: *C(=O)N1CCOCC1]
R^{a} are R^{z} are independently selected from hydrogen, C₁₋₆ alkyl, and X¹, or R^{a} and R^{z} combine to form a C₃₋₁₀ cycloalkyl, or a 3 to 10 membered heterocyclyl;
R^{d} is hydrogen, —CH₂NHC(O)X¹ or —CH₂NHC(O)X¹Q; and
Q is C₁₋₆ alkyl or hydrogen.

2. The compound according to claim 1 where in Z¹ is —O—, or a salt thereof.

3. The compound according to claim 1 wherein Z¹ is —NH—,
or a salt thereof.

4. The compound according to claim 1 wherein R^{d} is hydrogen, or a salt thereof.

5. The compound according to claim 1 wherein R^{d} is —CH₂NHC(O)X¹Q, or a salt thereof

6. The compound according to claim 1 wherein each R^{a} is independently selected from hydrogen and methyl,
or a salt thereof.

7. The compound according to claim 1 wherein each R^{c} is independently selected from hydrogen, hydroxyl, methyl, fluorine, and CHF₂, or two adjacent R^{c} combine to form spirocyclopropyl, or a salt thereof.

8. The compound according to claim 1 wherein Z is hydrogen, or a salt thereof.

9. The compound according to claim 1 wherein Z is —CH₂C(F₂)CHF₂, or a salt thereof.

10. The compound according to claim 1 wherein Z² is selected from —CH₂CH₂—, —CH₂—, —CH(CH₃)—, —CH₂CH(CH₃)-spirocyclopropyl(CH₂)₂—, and —(CH₂)₂spirocyclopropyl-, or salt thereof.

11. The compound according to claim 1 wherein Z² is absent, or a salt thereof.

12. The compound according to claim 1 wherein X is W1: 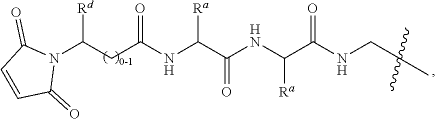
or a salt thereof.

13. The compound according to claim 1 wherein X is W2: 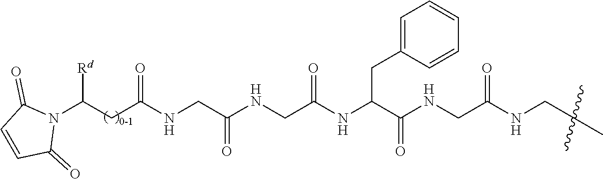
or a salt thereof.

14. The compound according to claim 1 wherein X is W3: 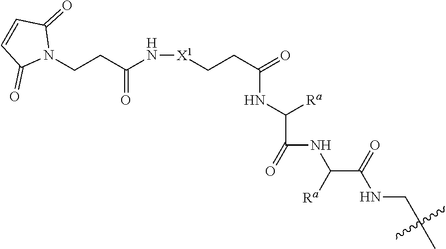
or a salt thereof.

15. The compound according to claim 1 wherein X is W4: 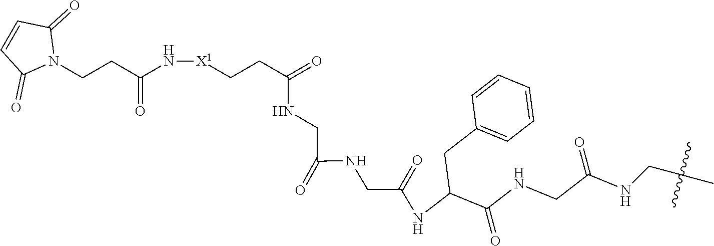
or a salt thereof.

16. The compound according to claim 1 wherein X is W5: 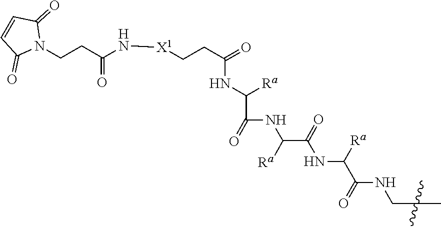
or a salt thereof.

17. The compound according to claim 1 wherein X is W6: 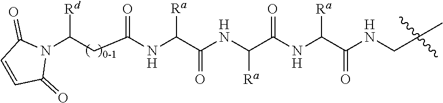
or a salt thereof.

18. The compound according to claim 1 wherein X is W7: 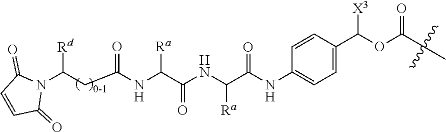
or a salt thereof.

19. The compound according to claim 1 wherein X is W8: 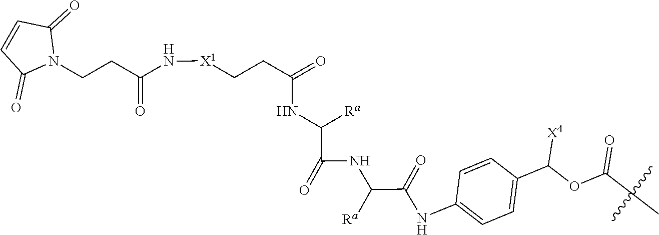
or a salt thereof.

20. The compound according to claim 1, represented by structural Formula II: 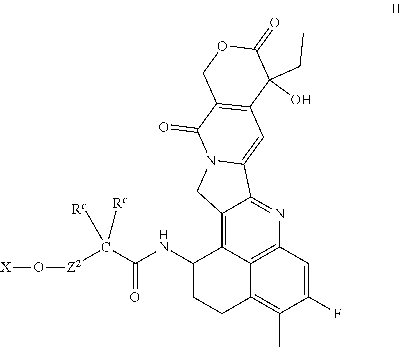
or a salt thereof.

21. The compound according to claim 1, represented by structural Formula III: 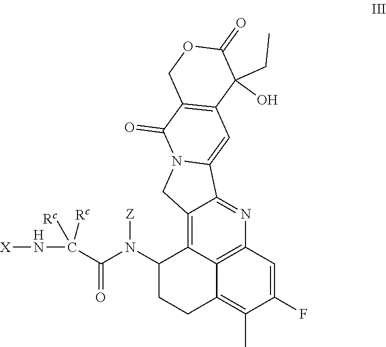
or a salt thereof.

22. A linker-drug compound, or a salt thereof selected from:
Structure

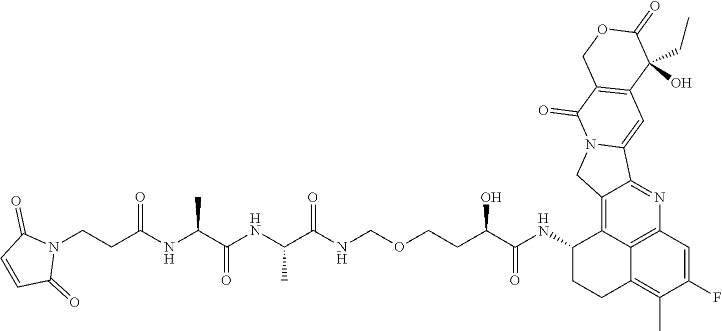 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)CCN1C(=O)C=CC1=O)CC3]

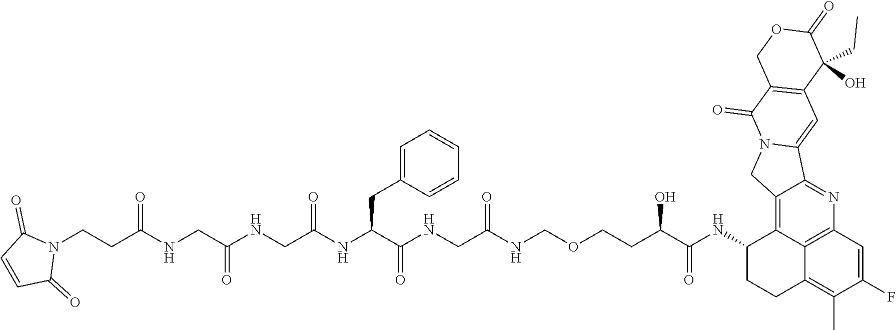 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)CCN1C(=O)C=CC1=O)CC3]

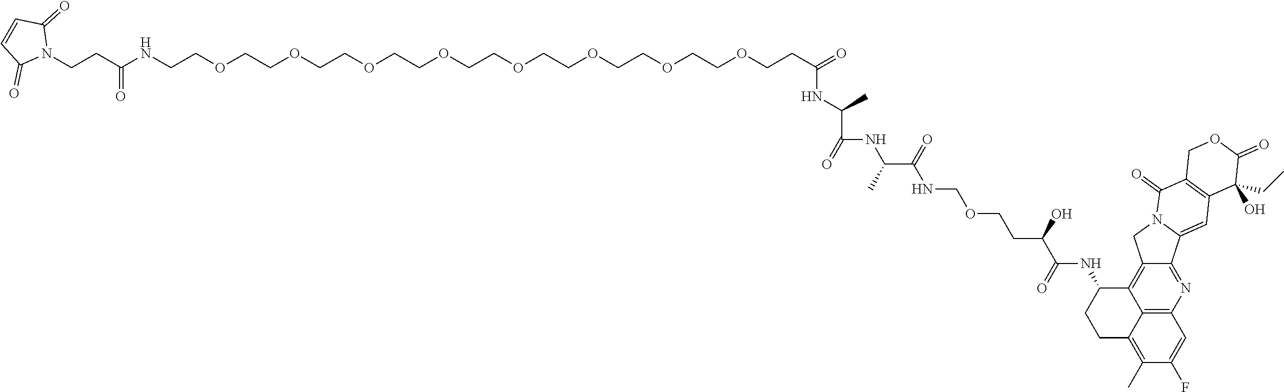 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)CCOCCOCCOCCOCCOCCOCCOCCOCCNC(=O)CCN1C(=O)C=CC1=O)CC3]

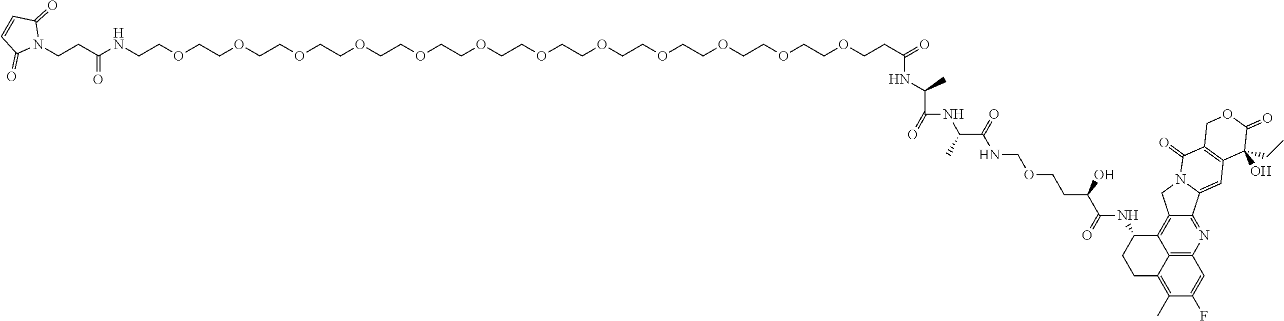 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)CCOCCOCCOCCOCCOCCOCCOCCOCCOCCOCCOCCOCCNC(=O)CCN1C(=O)C=CC1=O)CC3]

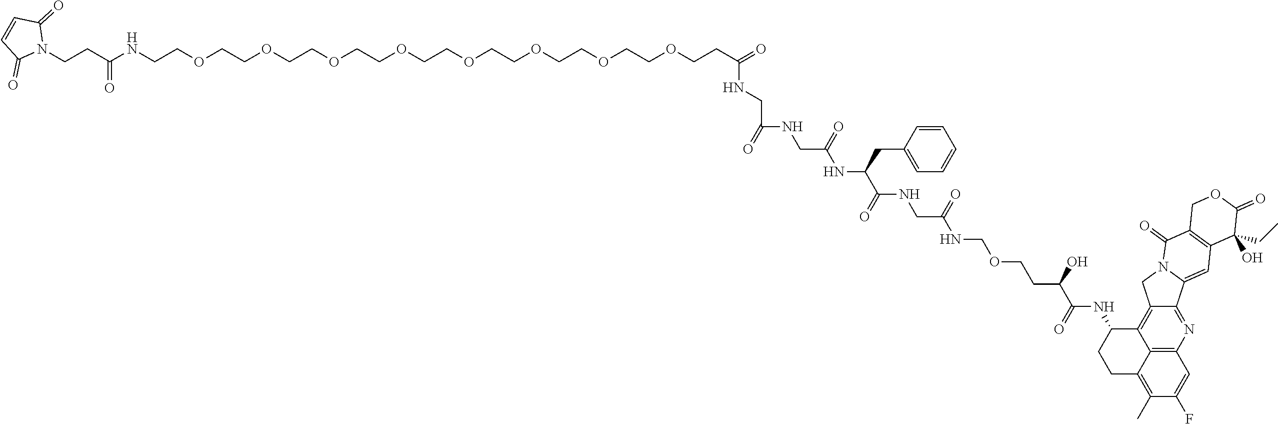 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)CCOCCOCCOCCOCCOCCOCCOCCOCCNC(=O)CCN1C(=O)C=CC1=O)CC3]

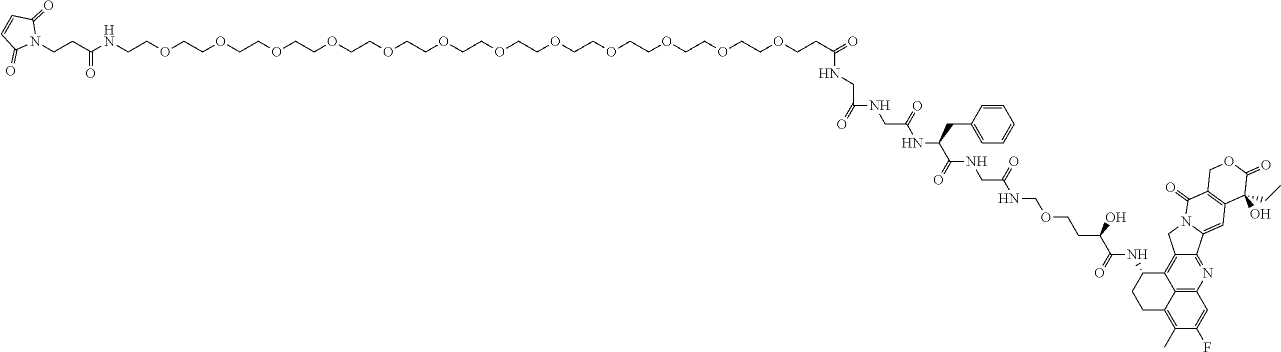 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)CCOCCOCCOCCOCCOCCOCCOCCOCCOCCOCCOCCOCCNC(=O)CCN1C(=O)C=CC1=O)CC3]

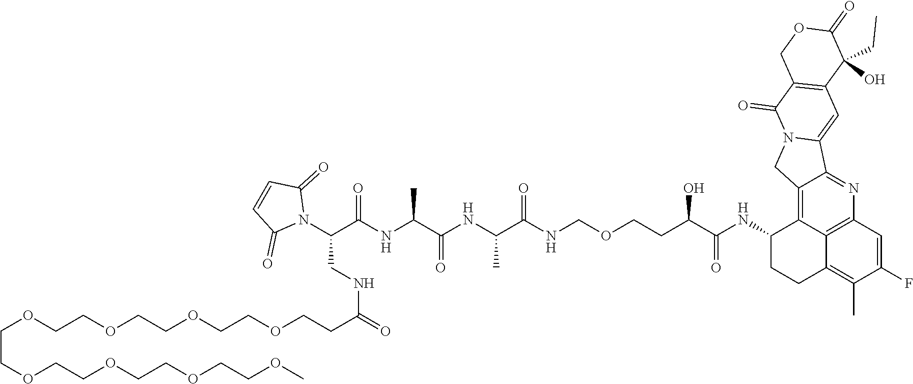 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)[C@H](CNC(=O)CCOCCOCCOCCOCCOCCOCCOCCOC)N1C(=O)C=CC1=O)CC3]

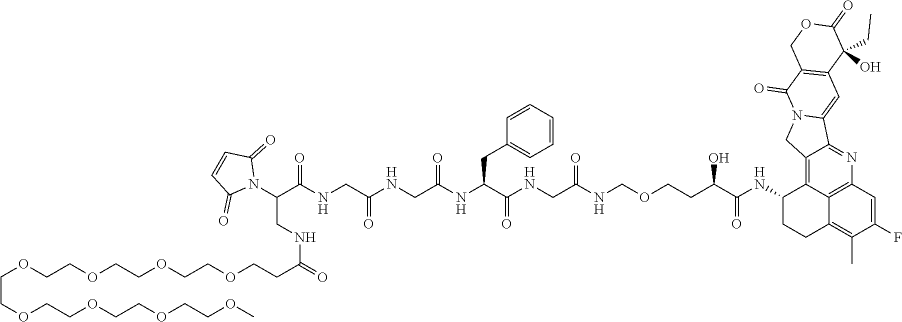 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)CNC(=O)[C@H](Cc1ccccc1)NC(=O)CNC(=O)CNC(=O)C(CNC(=O)CCOCCOCCOCCOCCOCCOCCOCCOC)N1C(=O)C=CC1=O)CC3]

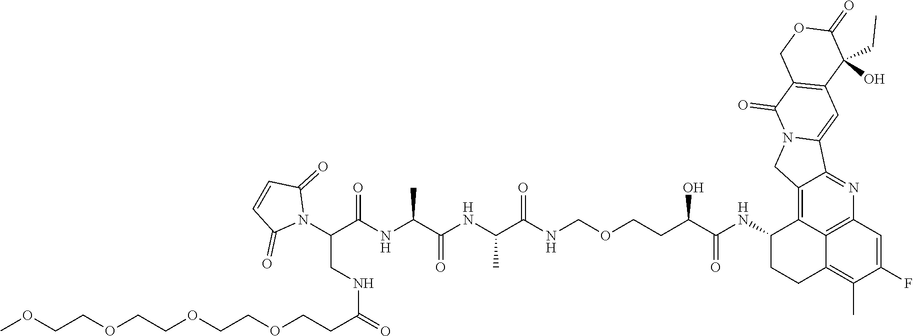 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C(CNC(=O)CCOCCOCCOCCOC)N1C(=O)C=CC1=O)CC3]

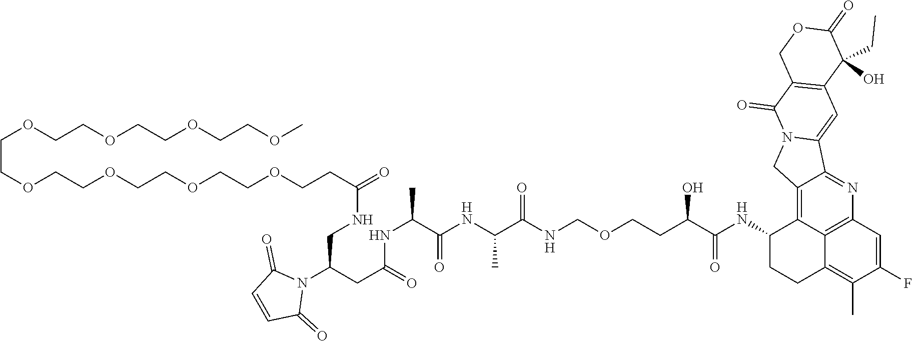 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C[C@H](CNC(=O)CCOCCOCCOCCOCCOCCOCCOCCOC)N1C(=O)C=CC1=O)CC3]

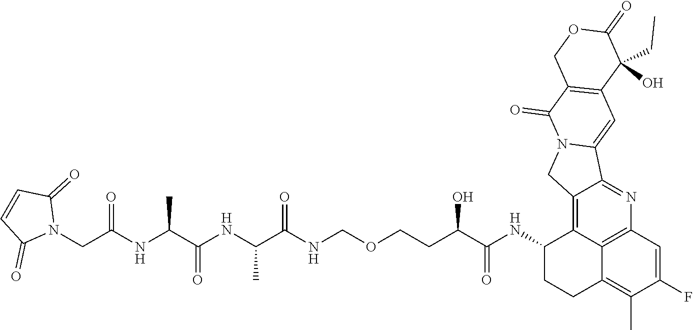 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)CN1C(=O)C=CC1=O)CC3]

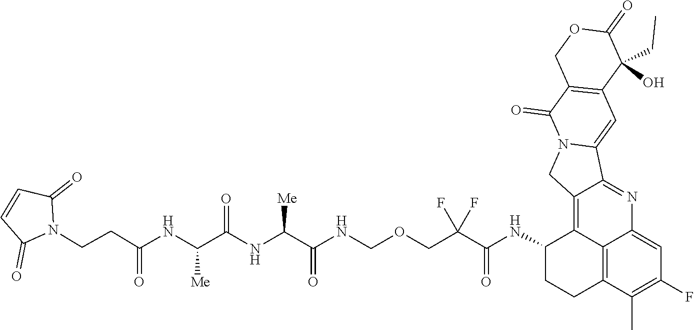 [SMILES: CC[C@@]1(O)C(=O)OCc2c1cc1n(c2=O)Cc2c-1nc1cc(F)c(C)c3c1c2[C@@H](NC(=O)C(F)(F)COCNC(=O)[C@H](C)NC(=O)[C@H](C)NC(=O)CCN1C(=O)C=CC1=O)CC3]

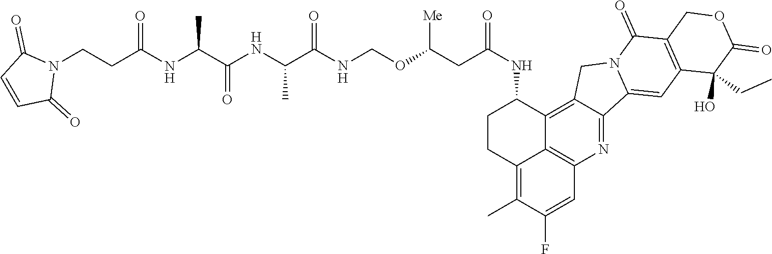 [SMILES: CC[C@@]1(O)C(=O)OCc2c1cc1n(c2=O)Cc2c-1nc1cc(F)c(C)c3c1c2[C@@H](NC(=O)C[C@@H](C)OCNC(=O)[C@H](C)NC(=O)[C@H](C)NC(=O)CCN1C(=O)C=CC1=O)CC3]

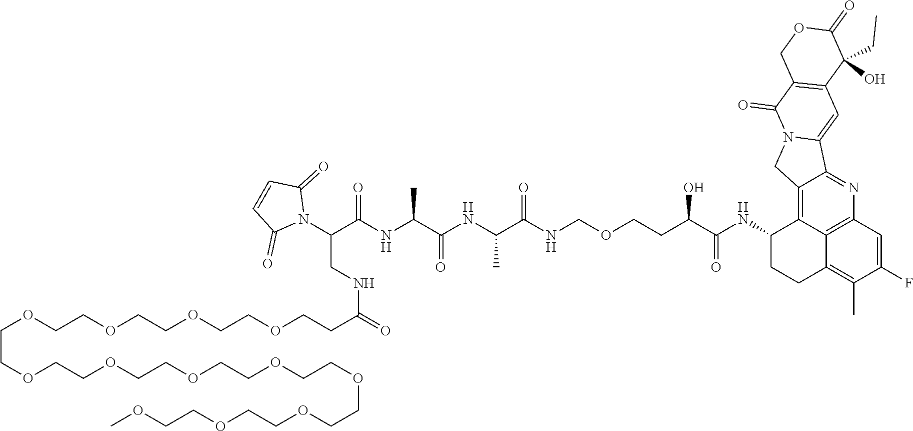 [SMILES: CC[C@@]1(O)C(=O)OCc2c1cc1n(c2=O)Cc2c-1nc1cc(F)c(C)c3c1c2[C@@H](NC(=O)[C@H](O)CCOCNC(=O)[C@H](C)NC(=O)[C@H](C)NC(=O)C(CNC(=O)CCOCCOCCOCCOCCOCCOCCOCCOCCOCCOCCOCCOC)N1C(=O)C=CC1=O)CC3]

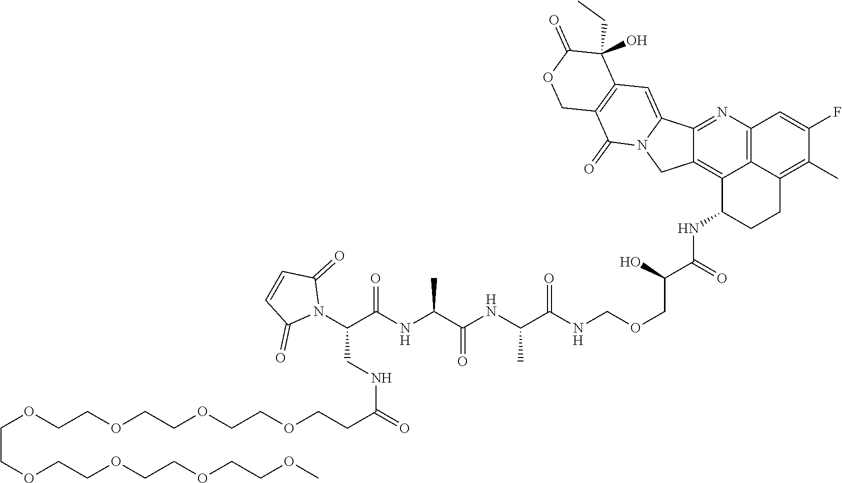 [SMILES: CC[C@@]1(O)C(=O)OCc2c1cc1n(c2=O)Cc2c-1nc1cc(F)c(C)c3c1c2[C@@H](NC(=O)[C@H](O)COCNC(=O)[C@H](C)NC(=O)[C@H](C)NC(=O)[C@H](CNC(=O)CCOCCOCCOCCOCCOCCOCCOCCOC)N1C(=O)C=CC1=O)CC3]

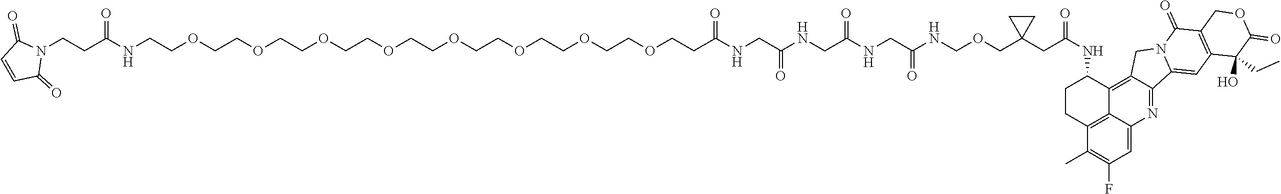 [SMILES: CC[C@@]1(O)C(=O)OCc2c1cc1n(c2=O)Cc2c-1nc1cc(F)c(C)c3c1c2[C@@H](NC(=O)CC1(COCNC(=O)CNC(=O)CNC(=O)CNC(=O)CCOCCOCCOCCOCCOCCOCCOCCOCCNC(=O)CCN2C(=O)C=CC2=O)CC1)CC3]

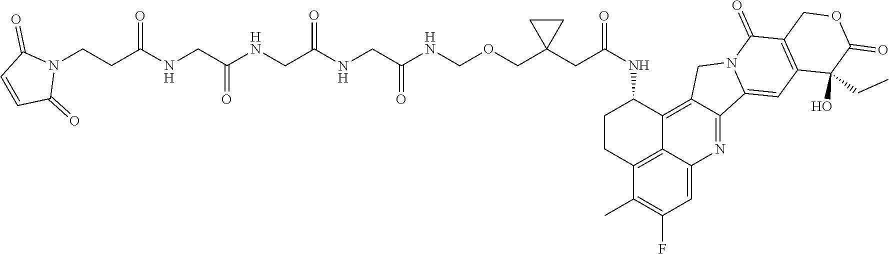 [SMILES: CC[C@@]1(O)C(=O)OCc2c1cc1n(c2=O)Cc2c-1nc1cc(F)c(C)c3c1c2[C@@H](NC(=O)CC1(COCNC(=O)CNC(=O)CNC(=O)CNC(=O)CCN2C(=O)C=CC2=O)CC1)CC3]

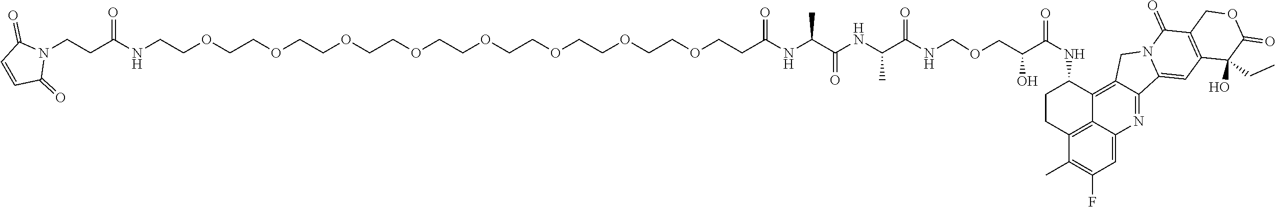 [SMILES: CC[C@@]1(O)C(=O)OCc2c1cc1n(c2=O)Cc2c-1nc1cc(F)c(C)c3c1c2[C@@H](NC(=O)[C@H](O)COCNC(=O)[C@H](C)NC(=O)[C@H](C)NC(=O)CCOCCOCCOCCOCCOCCOCCOCCOCCNC(=O)CCN1C(=O)C=CC1=O)CC3]

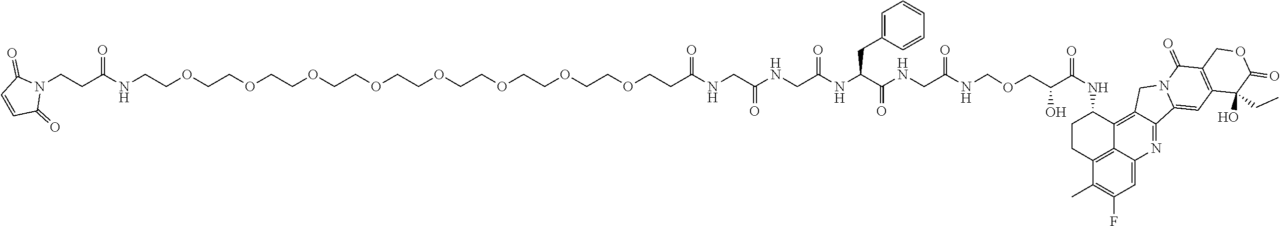 [SMILES: CC[C@@]1(O)C(=O)OCc2c1cc1n(c2=O)Cc2c-1nc1cc(F)c(C)c3c1c2[C@@H](NC(=O)[C@H](O)COCNC(=O)CNC(=O)[C@H](Cc1ccccc1)NC(=O)CNC(=O)CNC(=O)CCOCCOCCOCCOCCOCCOCCOCCOCCNC(=O)CCN1C(=O)C=CC1=O)CC3]

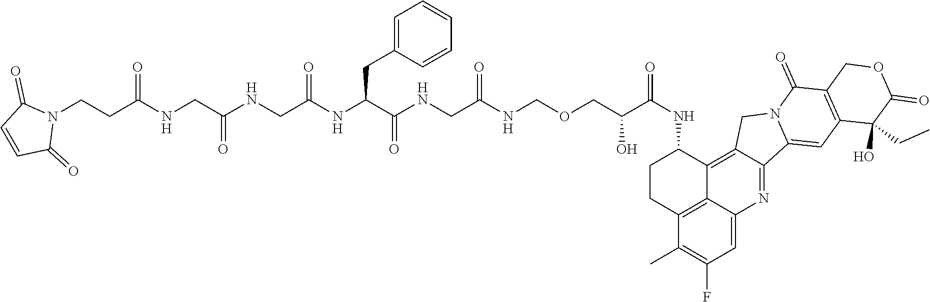 [SMILES: CC[C@@]1(O)C(=O)OCc2c1cc1n(c2=O)Cc2c-1nc1cc(F)c(C)c3c1c2[C@@H](NC(=O)[C@H](O)COCNC(=O)CNC(=O)[C@H](Cc1ccccc1)NC(=O)CNC(=O)CNC(=O)CCN1C(=O)C=CC1=O)CC3]

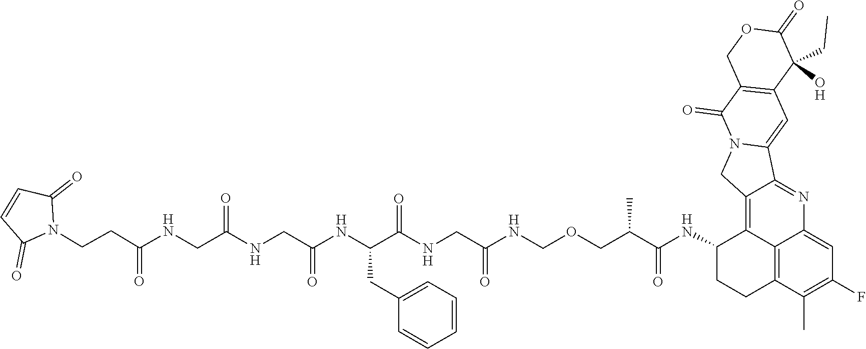 [SMILES: CC[C@@]1(O)C(=O)OCc2c1cc1n(c2=O)Cc2c-1nc1cc(F)c(C)c3c1c2[C@@H](NC(=O)[C@@H](C)COCNC(=O)CNC(=O)[C@H](Cc1ccccc1)NC(=O)CNC(=O)CNC(=O)CCN1C(=O)C=CC1=O)CC3]

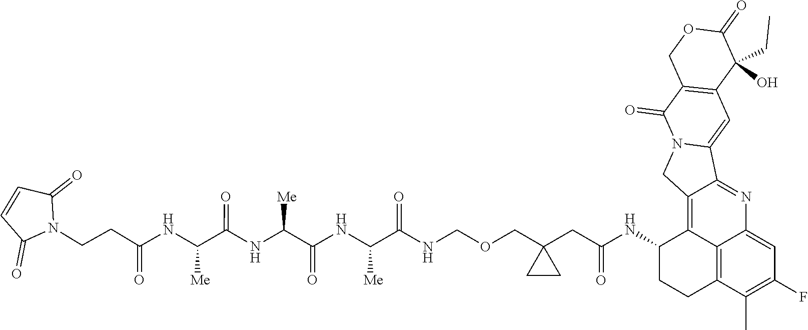 [SMILES: CC[C@@]1(O)C(=O)OCc2c1cc1n(c2=O)Cc2c-1nc1cc(F)c(C)c3c1c2[C@@H](NC(=O)CC1(COCNC(=O)[C@H](C)NC(=O)[C@H](C)NC(=O)[C@H](C)NC(=O)CCN2C(=O)C=CC2=O)CC1)CC3]

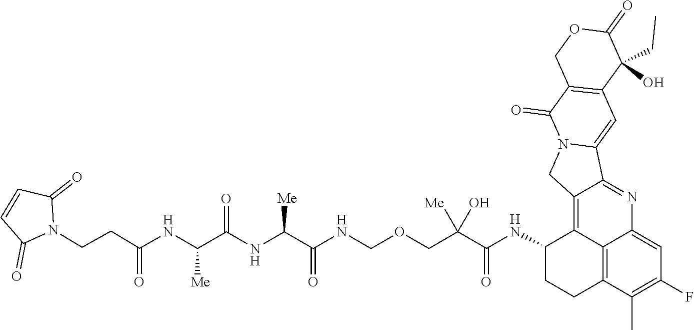 [SMILES: CC[C@@]1(O)C(=O)OCc2c1cc1n(c2=O)Cc2c-1nc1cc(F)c(C)c3c1c2[C@@H](NC(=O)C(C)(O)COCNC(=O)[C@H](C)NC(=O)[C@H](C)NC(=O)CCN1C(=O)C=CC1=O)CC3]

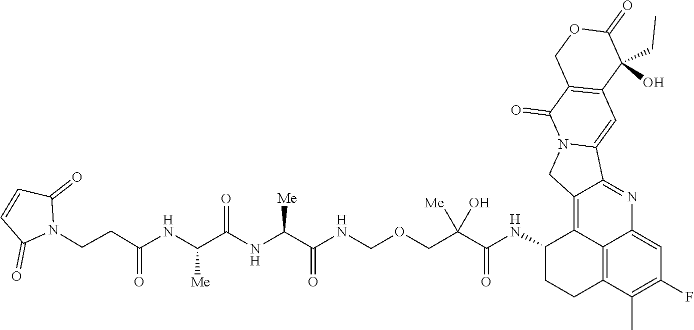 [SMILES: CC[C@@]1(O)C(=O)OCc2c1cc1n(c2=O)Cc2c-1nc1cc(F)c(C)c3c1c2[C@@H](NC(=O)C(C)(O)COCNC(=O)[C@H](C)NC(=O)[C@H](C)NC(=O)CCN1C(=O)C=CC1=O)CC3]

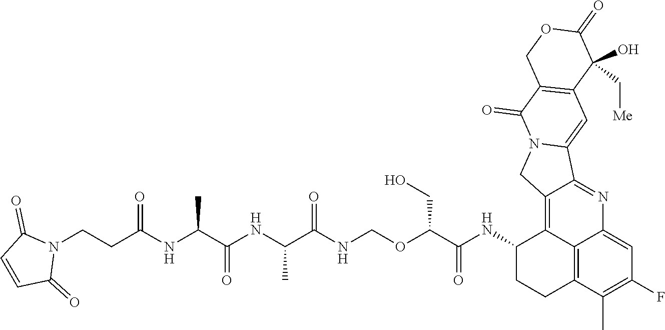 [SMILES: CC[C@@]1(O)C(=O)OCc2c1cc1n(c2=O)Cc2c-1nc1cc(F)c(C)c3c1c2[C@@H](NC(=O)[C@@H](CO)OCNC(=O)[C@H](C)NC(=O)[C@H](C)NC(=O)CCN1C(=O)C=CC1=O)CC3]

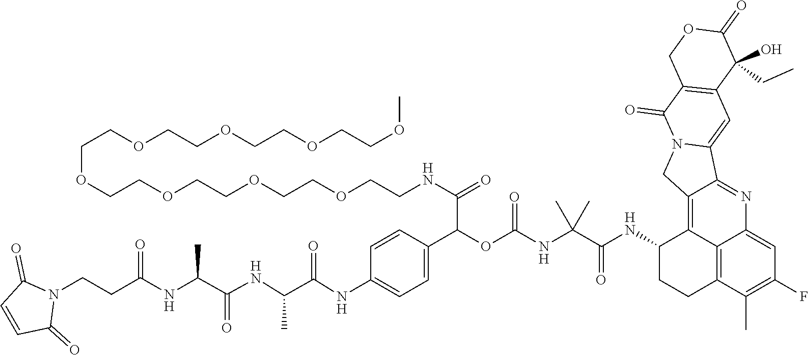 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OC(C(=O)NCCOCCOCCOCCOCCOCCOCCOCCOC)c1ccc(NC(=O)[C@H](C)NC(=O)[C@H](C)NC(=O)CCN2C(=O)C=CC2=O)cc1)CC3]

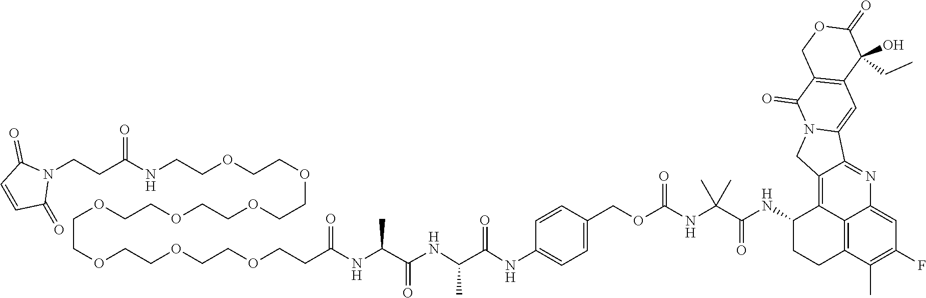 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)CCOCCOCCOCCOCCOCCOCCOCCOCCNC(=O)CCN2C(=O)C=CC2=O)cc1)CC3]

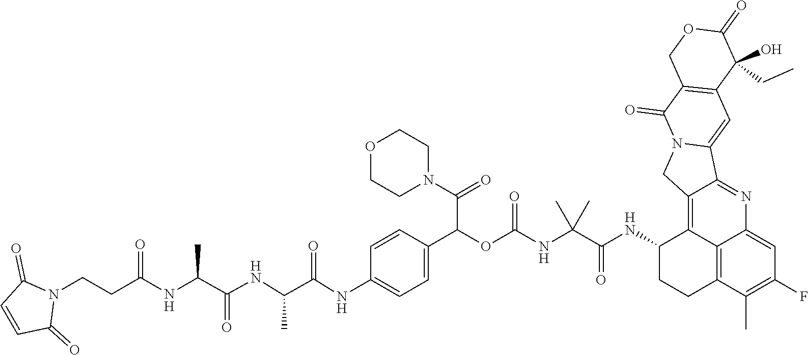 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OC(C(=O)N1CCOCC1)c1ccc(NC(=O)[C@H](C)NC(=O)[C@H](C)NC(=O)CCN2C(=O)C=CC2=O)cc1)CC3]

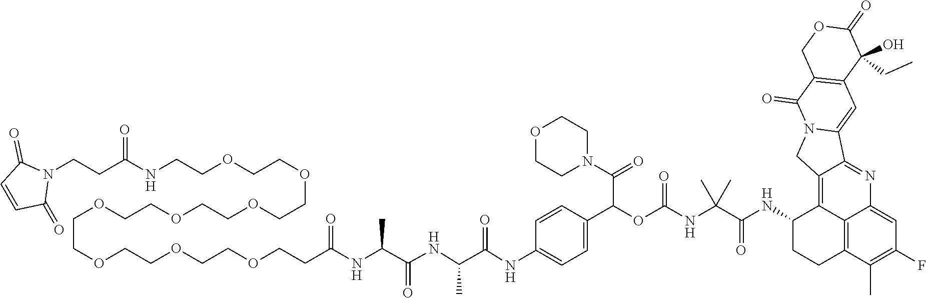 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OC(C(=O)N1CCOCC1)c1ccc(NC(=O)[C@H](C)NC(=O)[C@H](C)NC(=O)CCOCCOCCOCCOCCOCCOCCOCCOCCNC(=O)CCN2C(=O)C=CC2=O)cc1)CC3]

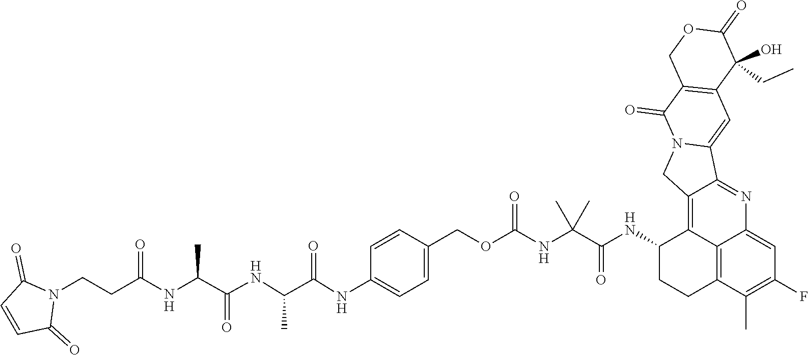 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)CCN2C(=O)C=CC2=O)cc1)CC3]

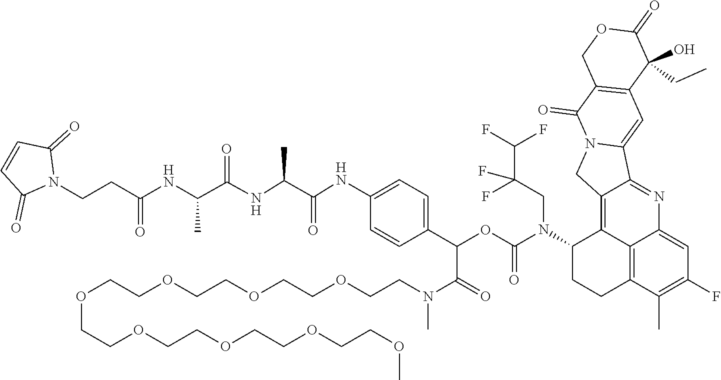 [SMILES: CC[C@@]1(O)C(=O)OCc2c1cc1n(c2=O)Cc2c-1nc1cc(F)c(C)c3c1c2[C@@H](N(CC(F)(F)C(F)F)C(=O)OC(C(=O)N(C)CCOCCOCCOCCOCCOCCOCCOCCOC)c1ccc(NC(=O)[C@H](C)NC(=O)[C@H](C)NC(=O)CCN2C(=O)C=CC2=O)cc1)CC3]

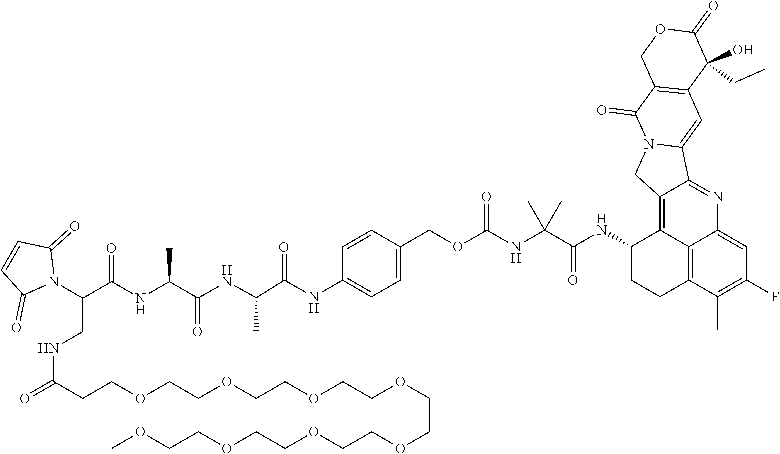 [SMILES: CC[C@@]1(O)C(=O)OCc2c1cc1n(c2=O)Cc2c-1nc1cc(F)c(C)c3c1c2[C@@H](NC(=O)C(C)(C)NC(=O)OCc1ccc(NC(=O)[C@H](C)NC(=O)[C@H](C)NC(=O)C(CNC(=O)CCOCCOCCOCCOCCOCCOCCOCCOC)N2C(=O)C=CC2=O)cc1)CC3]

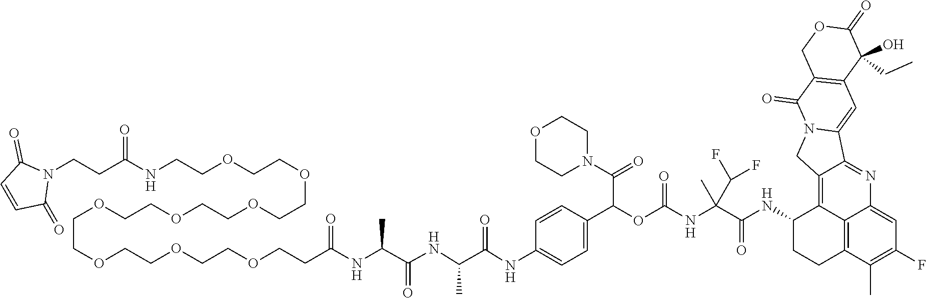 [SMILES: CC[C@@]1(O)C(=O)OCc2c1cc1n(c2=O)Cc2c-1nc1cc(F)c(C)c3c1c2[C@@H](NC(=O)C(C)(NC(=O)OC(C(=O)N1CCOCC1)c1ccc(NC(=O)[C@H](C)NC(=O)[C@H](C)NC(=O)CCOCCOCCOCCOCCOCCOCCOCCOCCNC(=O)CCN2C(=O)C=CC2=O)cc1)C(F)F)CC3]

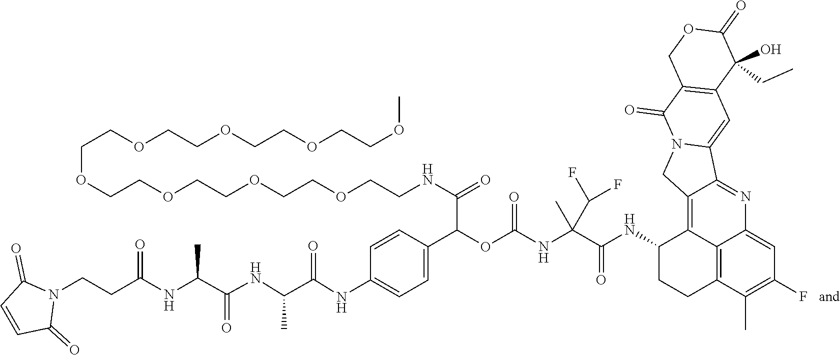 [SMILES: CC[C@@]1(O)C(=O)OCc2c1cc1n(c2=O)Cc2c-1nc1cc(F)c(C)c3c1c2[C@@H](NC(=O)C(C)(NC(=O)OC(C(=O)NCCOCCOCCOCCOCCOCCOCCOCCOC)c1ccc(NC(=O)[C@H](C)NC(=O)[C@H](C)NC(=O)CCN2C(=O)C=CC2=O)cc1)C(F)F)CC3]

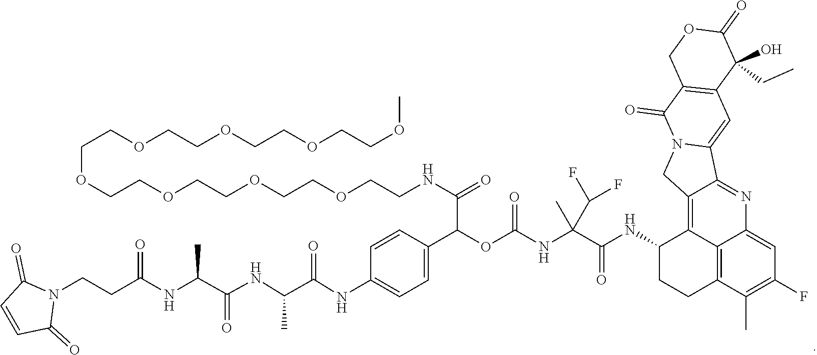 [SMILES: CC[C@@]1(O)C(=O)OCc2c1cc1n(c2=O)Cc2c-1nc1cc(F)c(C)c3c1c2[C@@H](NC(=O)C(C)(NC(=O)OC(C(=O)NCCOCCOCCOCCOCCOCCOCCOCCOC)c1ccc(NC(=O)[C@H](C)NC(=O)[C@H](C)NC(=O)CCN2C(=O)C=CC2=O)cc1)C(F)F)CC3]

23. A compound of structural Formula IV, or a pharmaceutically acceptable salt thereof: 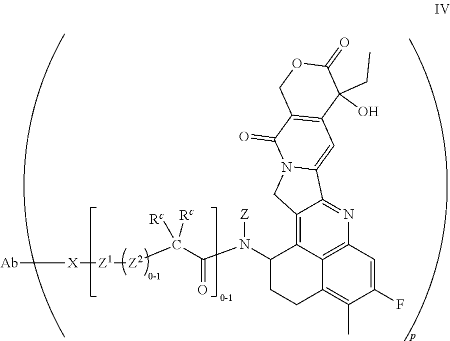
wherein Ab is an antibody;
p is an integer from 1 to 24;
Z is selected from hydrogen and —CH₂C(R^{x})(R^{y})CHF₂;
Z¹ is selected from —NH— and —O—;
Z² is absent or selected from —CR^{b}R^{b}—, —CH₂CR^{b}R^{b}—, and —CR^{b}R^{b}CH₂—;
each R^{b} is independently selected from hydrogen, —C₁₋₆ alkyl, and hydroxyl;
or two adjacent R^{b} combine to form a spirocycloalkyl;
each R^{c} is independently selected from hydrogen, —C₁₋₆ alkyl, halogen, and hydroxyl;
or two adjacent R^{c} combine to form a spirocycloalkyl;
R^{x} and R^{y} are independently selected from hydrogen, C₁₋₆ alkyl, C₁₋₃ haloalkyl, halogen, hydroxyl, and —C₁₋₆ alkyl-OH;
or R^{x} and R^{y} combine to form a C₃₋₆cycloalkyl, or spirocycloalkyl;
X is a linking group selected from: 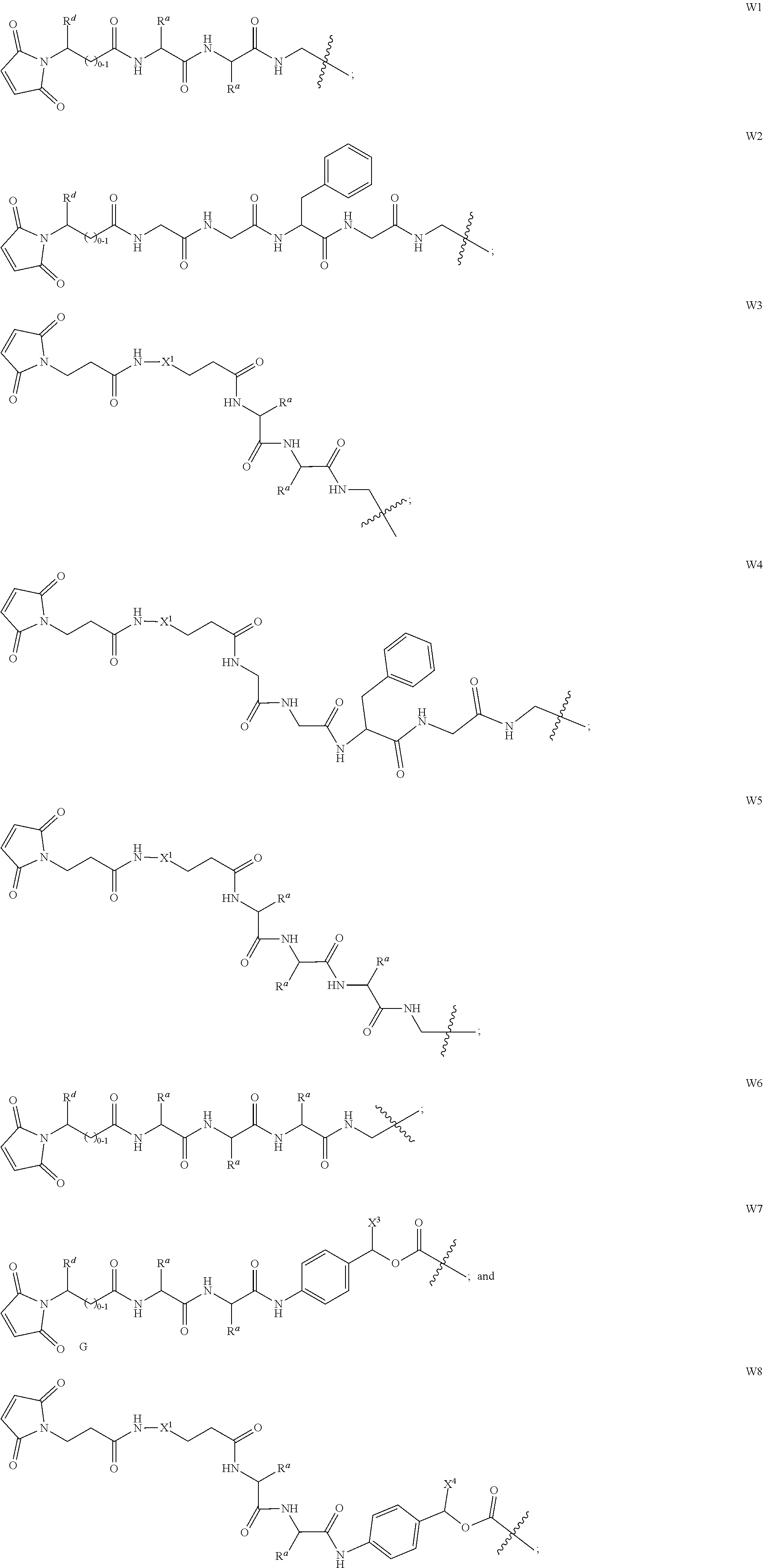 [symbol]
represents point of attachment to structural Formula I;
X¹ is a polyethylene glycol (PEG);
X² is —NR^{a}X¹—, or 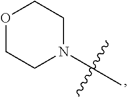 [SMILES: *N1CCOCC1]
X³ is hydrogen or —C(O)X²,
X⁴ is hydrogen or 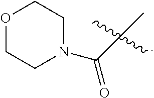 [SMILES: *C(=O)N1CCOCC1]
R^{a} is selected from hydrogen and C₁₋₆ alkyl; and
R^{d} is hydrogen or —CH₂NHC(O)X¹Q,
or a pharmaceutically acceptable salt thereof.

24. The compound according to claim 23, or a pharmaceutically acceptable salt there of which is: 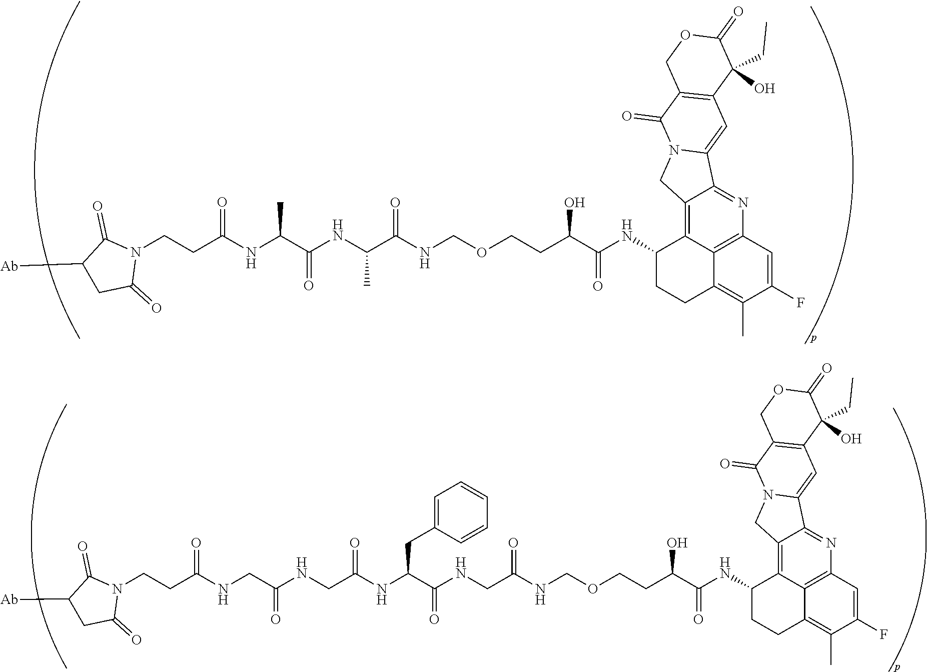 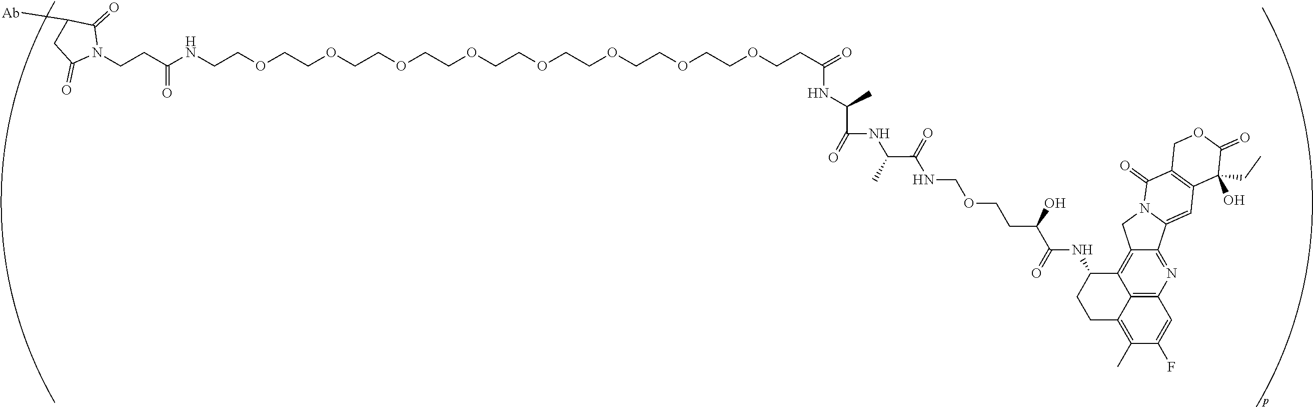 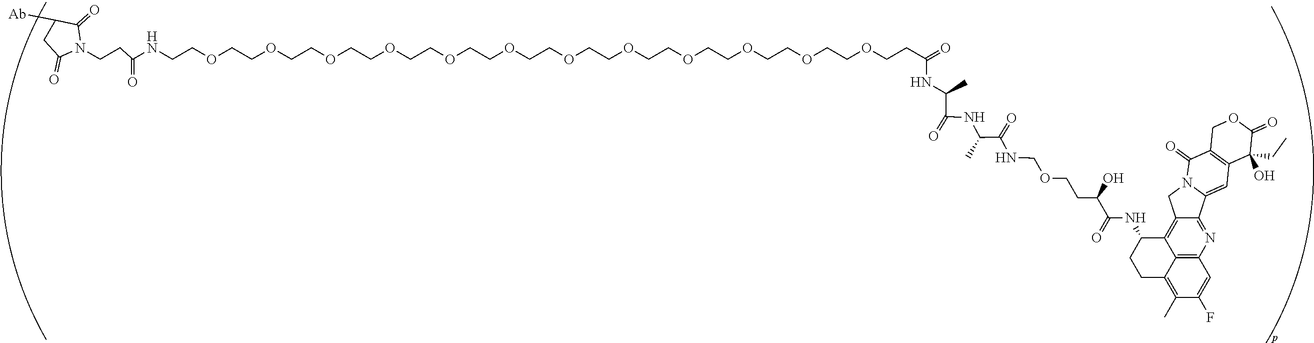 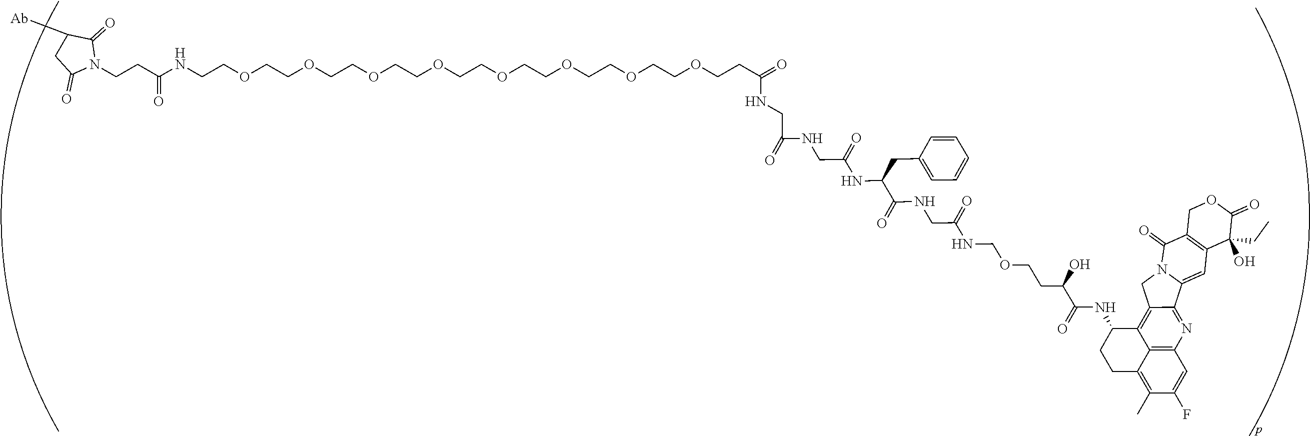 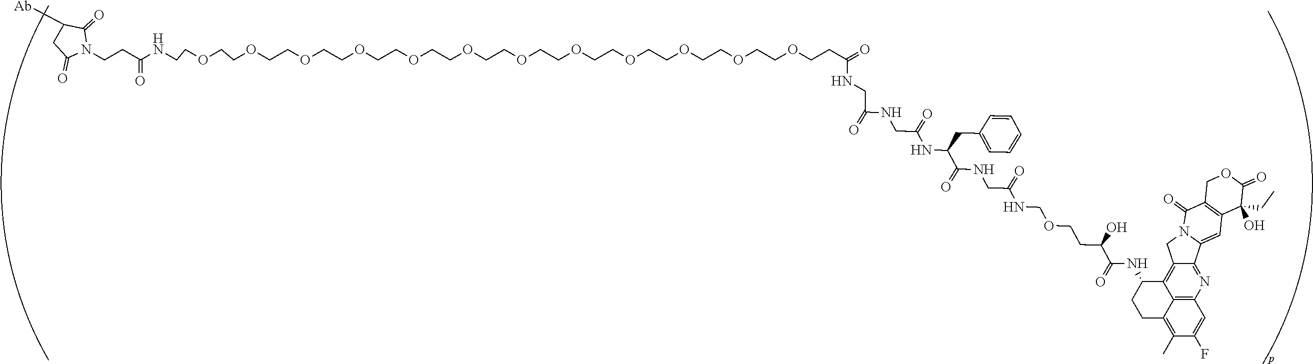 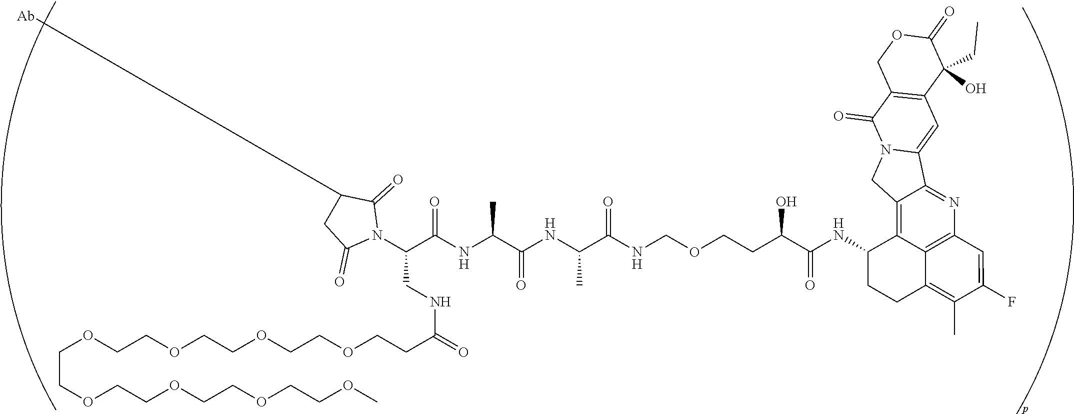 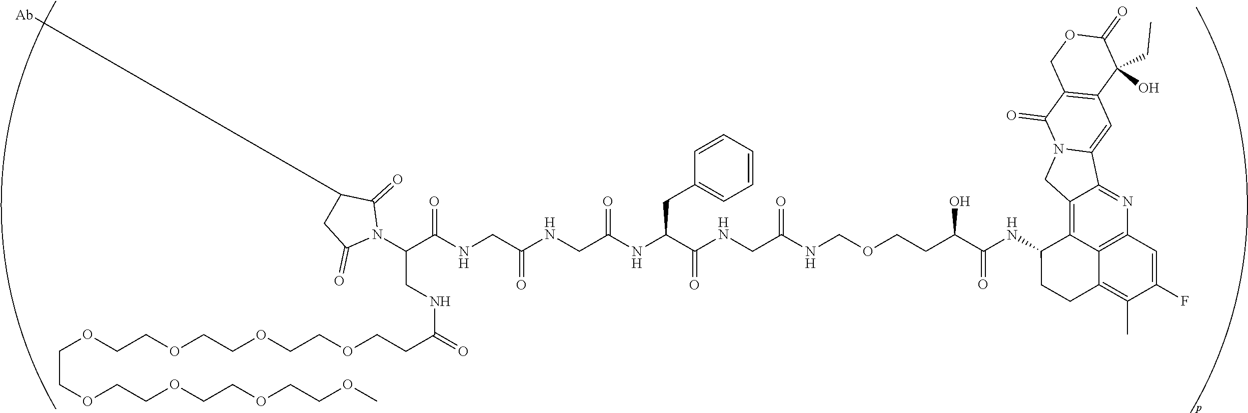 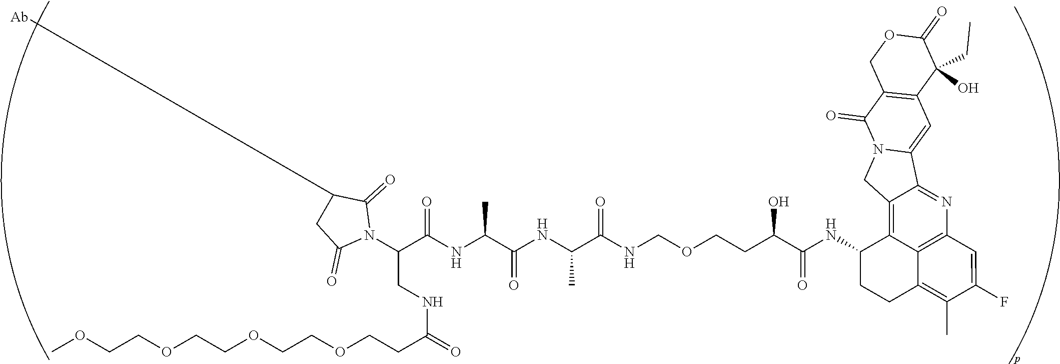 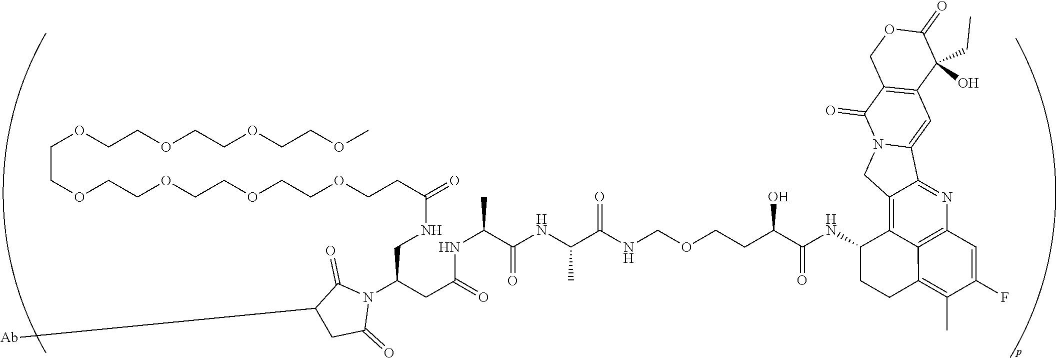 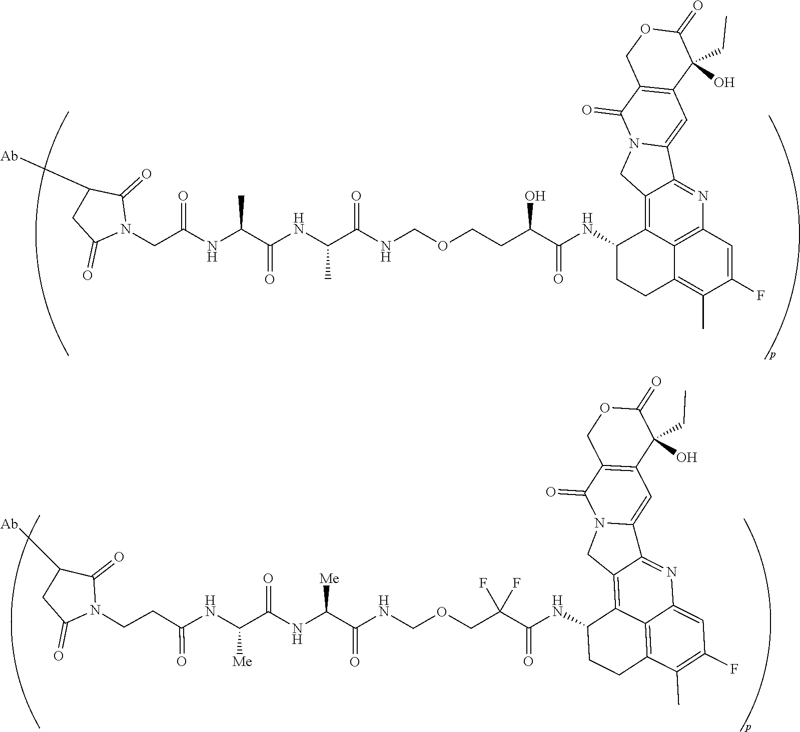 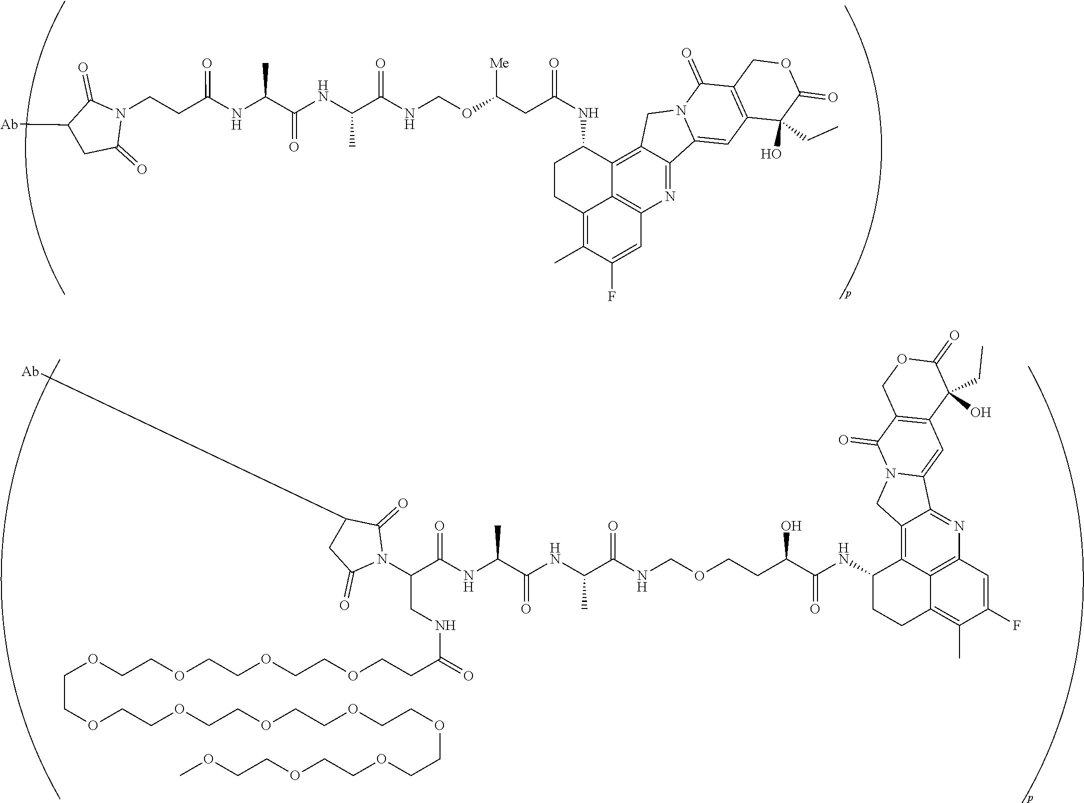 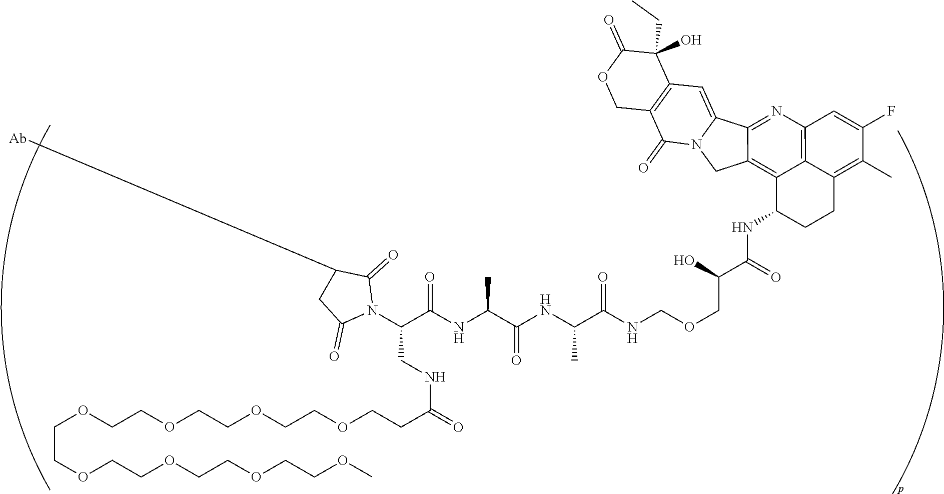 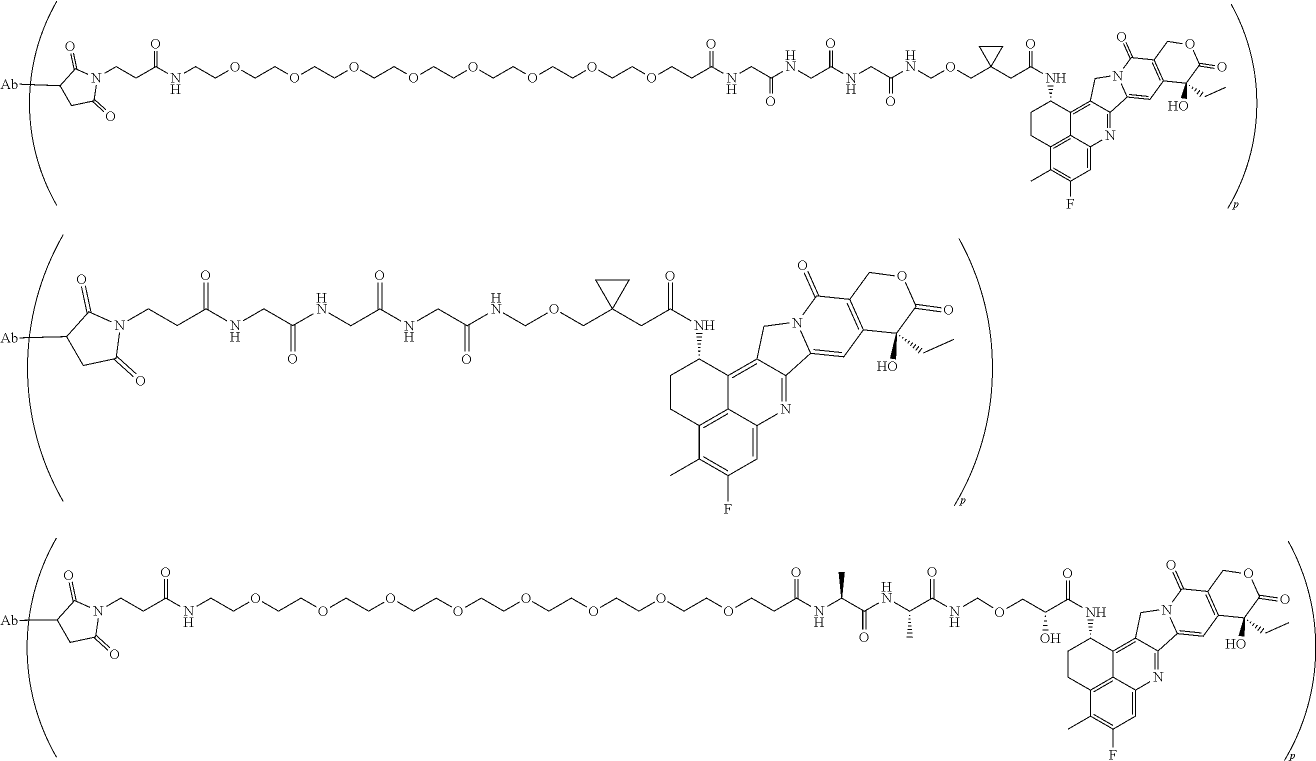 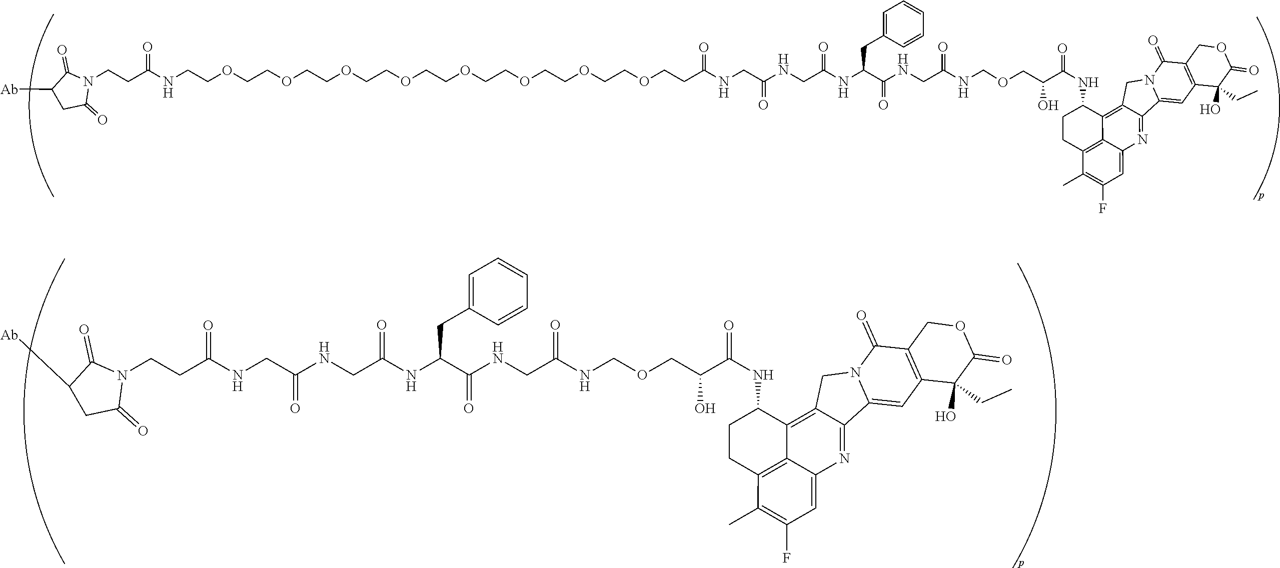 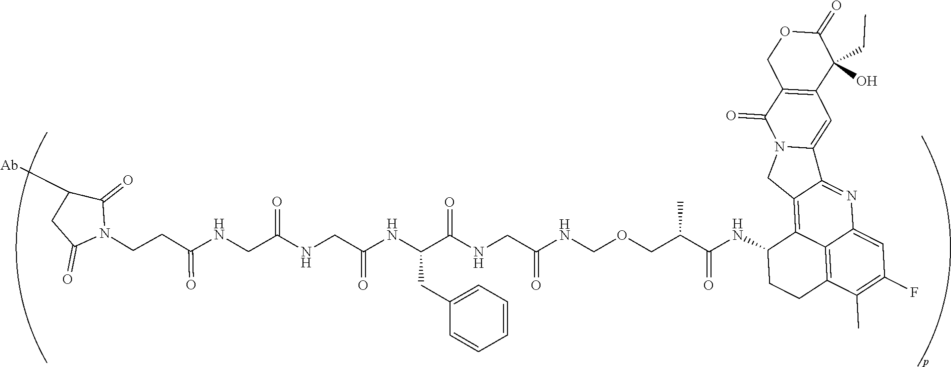 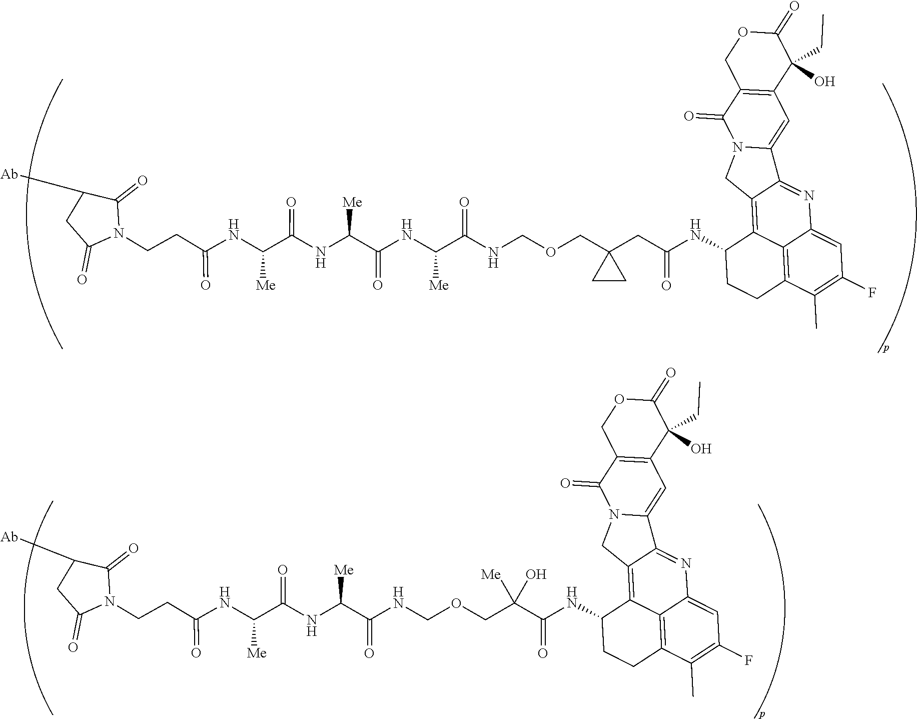 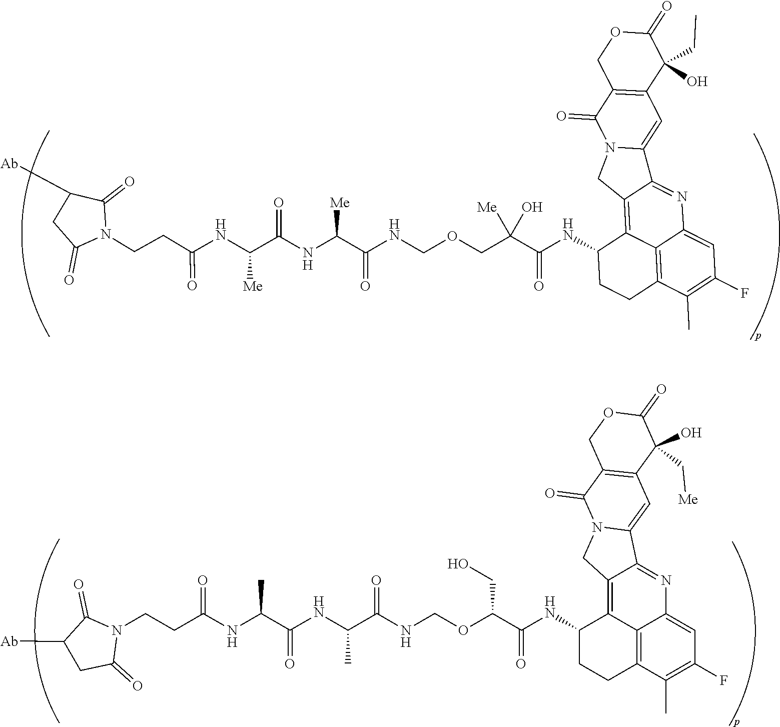 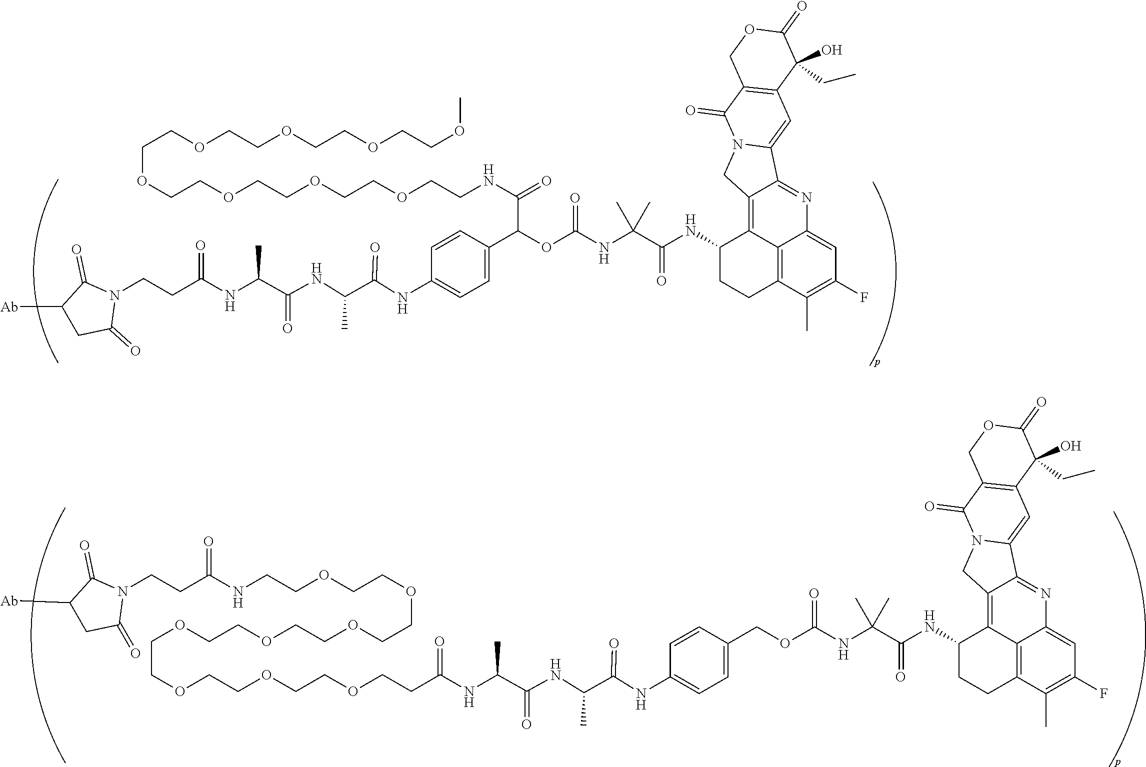 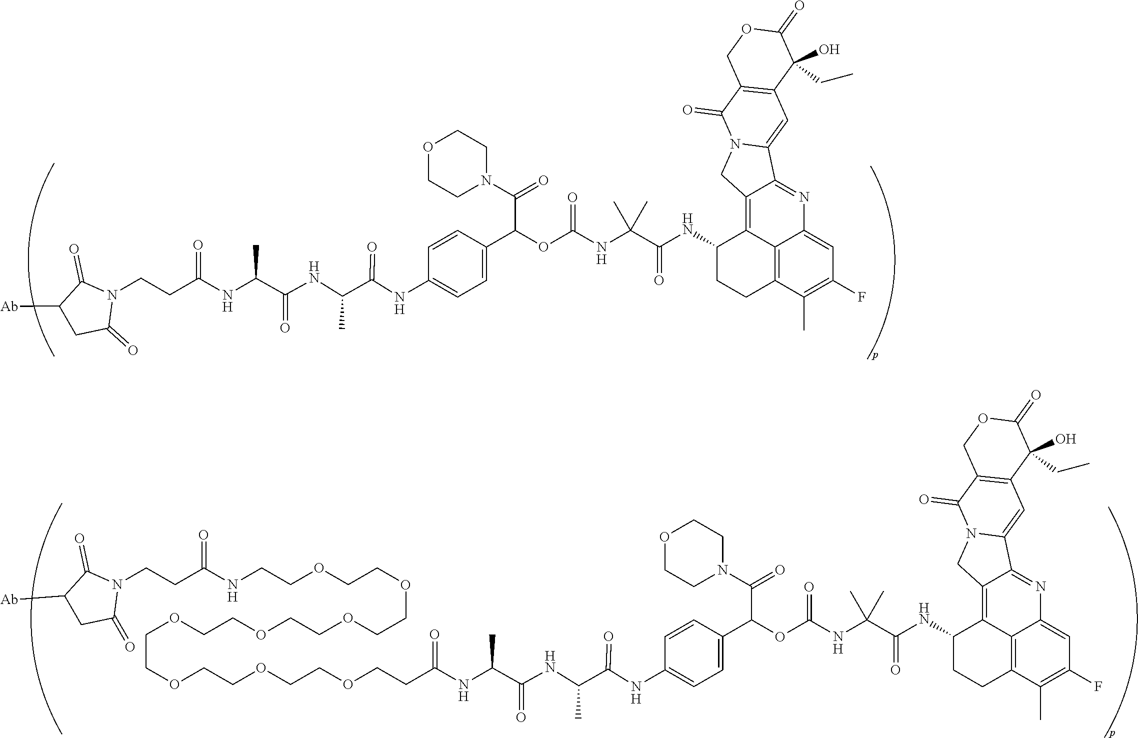 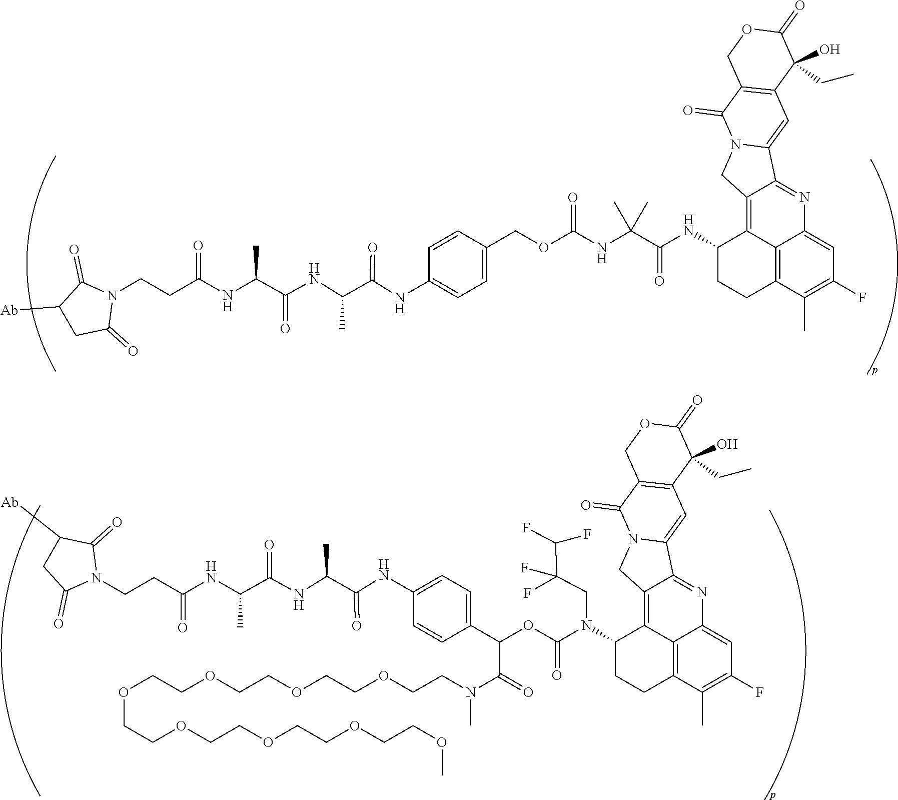 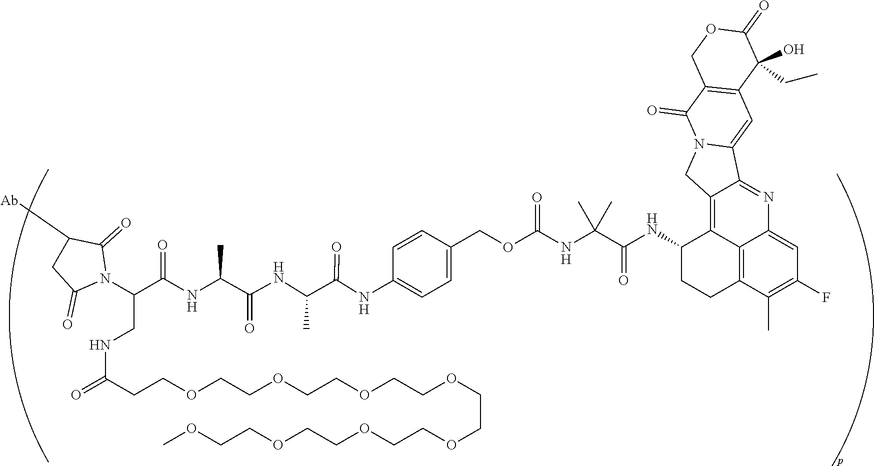 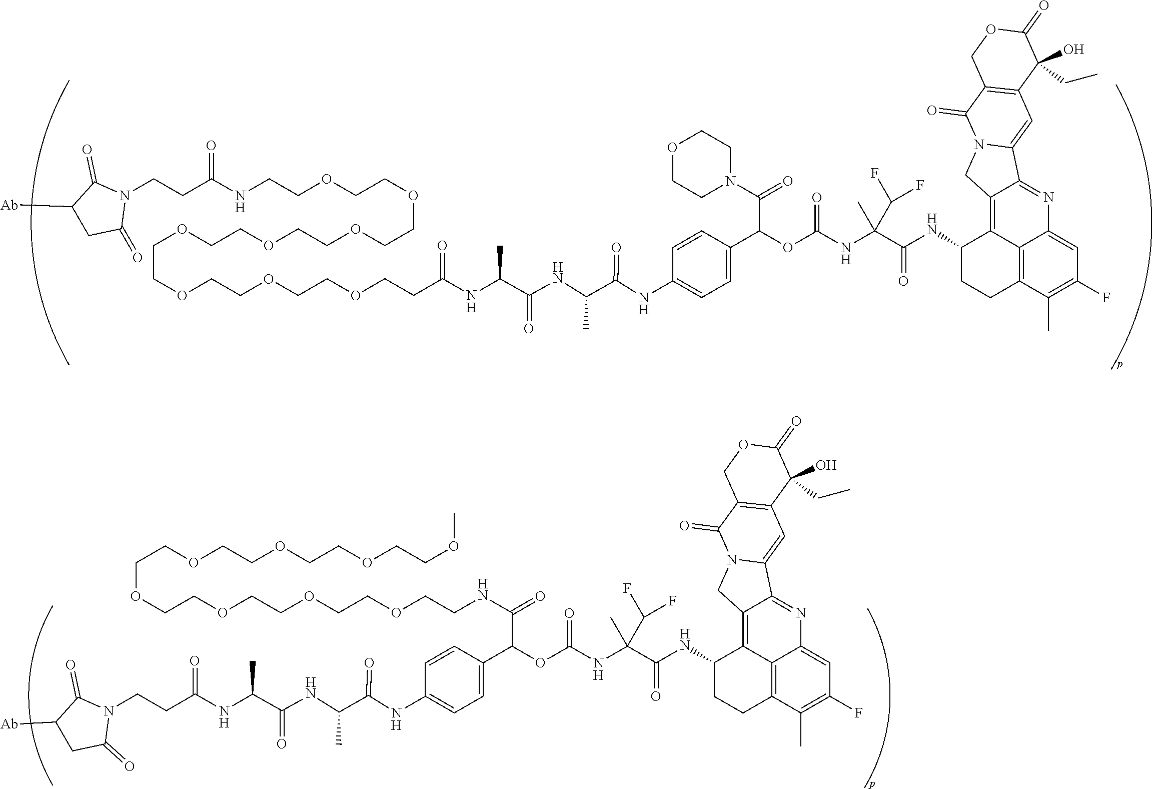 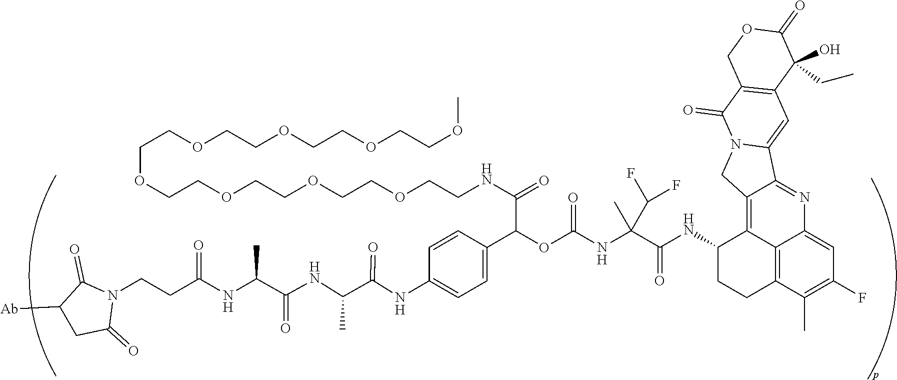
wherein Ab is an antibody and p is an integer 1 to 12.

25. A compound of structural formula V: 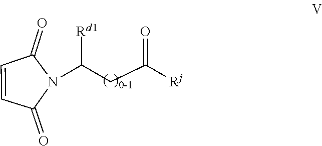
wherein:
R^{j} is selected from OH, —NH₂, —NHR^{k}R^{g}, —NHR^{k}NH(CH₂)ₙOR^{q}, —NHR^{k}NH(CH₂)ₙOC(O)CH₃, —NHX^{1a}(CH₂)ₙC(O)R^{k}NHCH₂OC(O)CH₃, —NHR^{k}NHR^{L}, —NHX^{1a}R^{k}NHR^{L}, and —NHCH₂O(CH₂)ₙCH(OH)C(O)OH;
R^{g} is COOH or CONH₂;
R^{k} is an amino acid residue of up to 10 amino acids;
R^{L} is selected from: 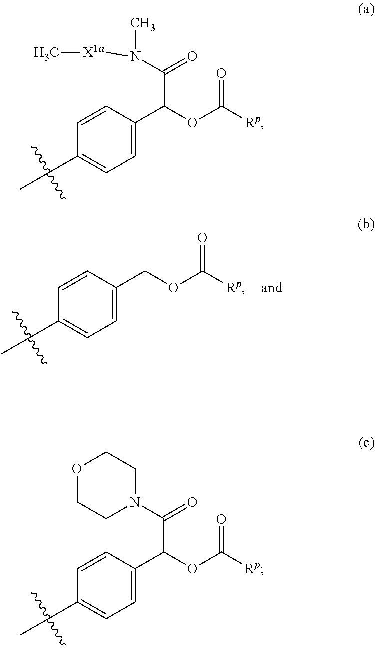
R^{p} is selected from NH₂ and 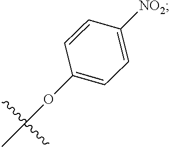 [SMILES: *Oc1ccc([N+](=O)[O-])cc1]
R^{q} is hydrogen or C₁₋₆ alkyl;
X^{1a} is a PEG of 1 to 24 —CH₂CH₂O— subunits;
R^{d1} is hydrogen, —CH₂NHC(O)X^{1a}Q or —CH₂NHC(O)X²¹Q;
Q is C₁₋₆ alkyl or H;
X²¹ is selected from a PEG of 1 to 24 —CH₂CH₂O— subunits, PEG-amino sugar, and p-aminobenzylcarbonyl and
n is 1, 2, 3, or 4.

26. A compound of claim 25, selected from: 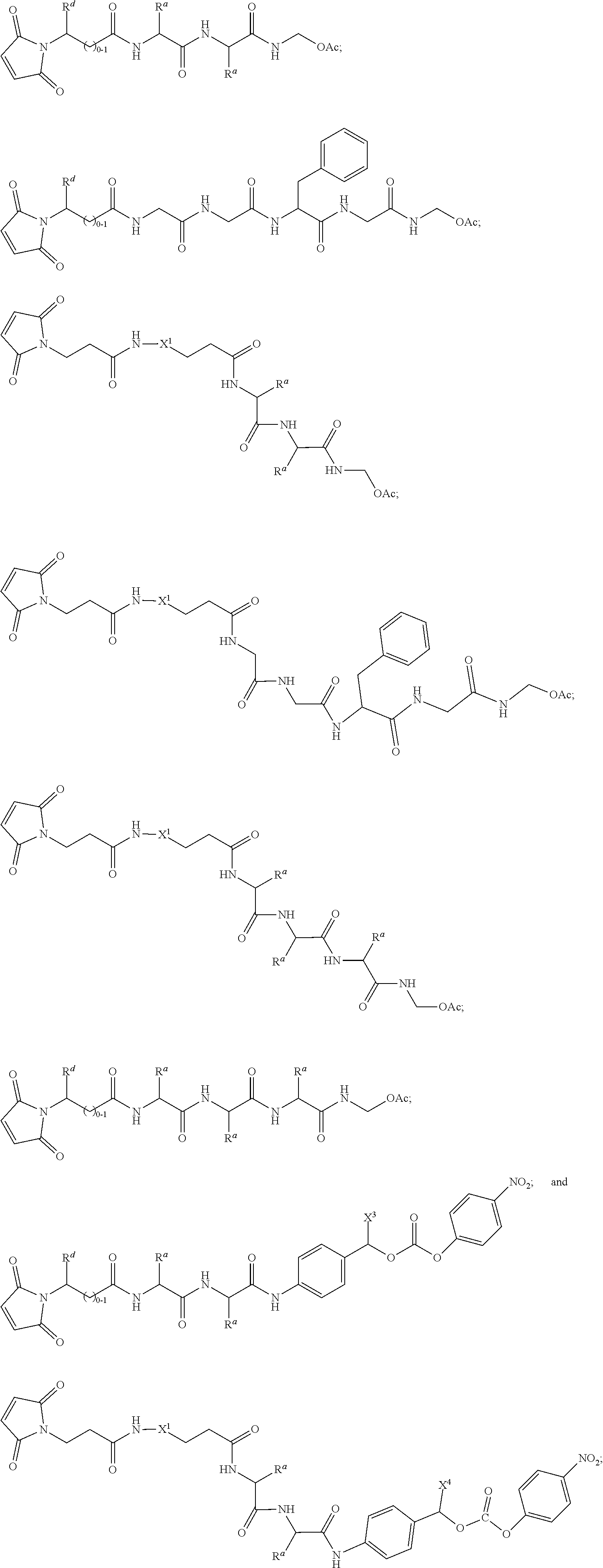
or a pharmaceutically acceptable salt or solvate thereof, wherein
X¹ is a Polyethylene Glycol (PEG);
X² is —NR^{a}X¹—, or 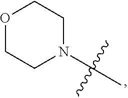 [SMILES: *N1CCOCC1]
X³ is hydrogen or —C(O)X²,
X⁴ is hydrogen or 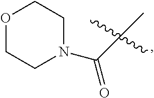 [SMILES: *C(=O)N1CCOCC1]
R^{a} is selected from hydrogen and C₁₋₆ alkyl; and
R^{d} is hydrogen or —CH₂NHC(O)X¹Q.

27. The compound according to claim 25, selected from: 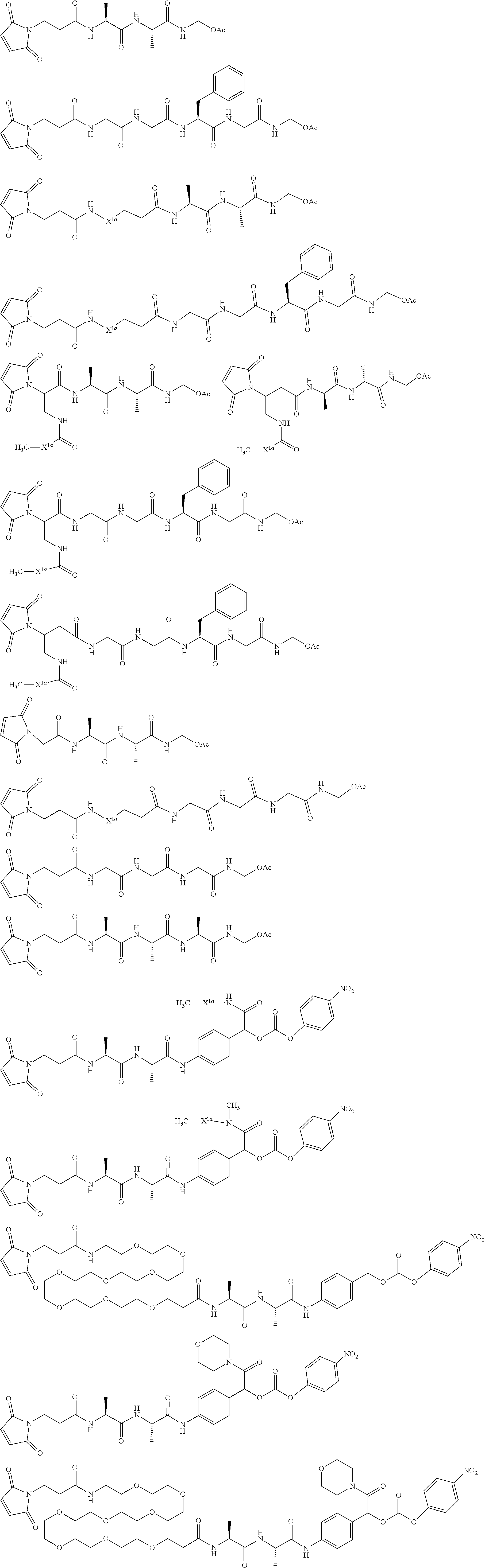 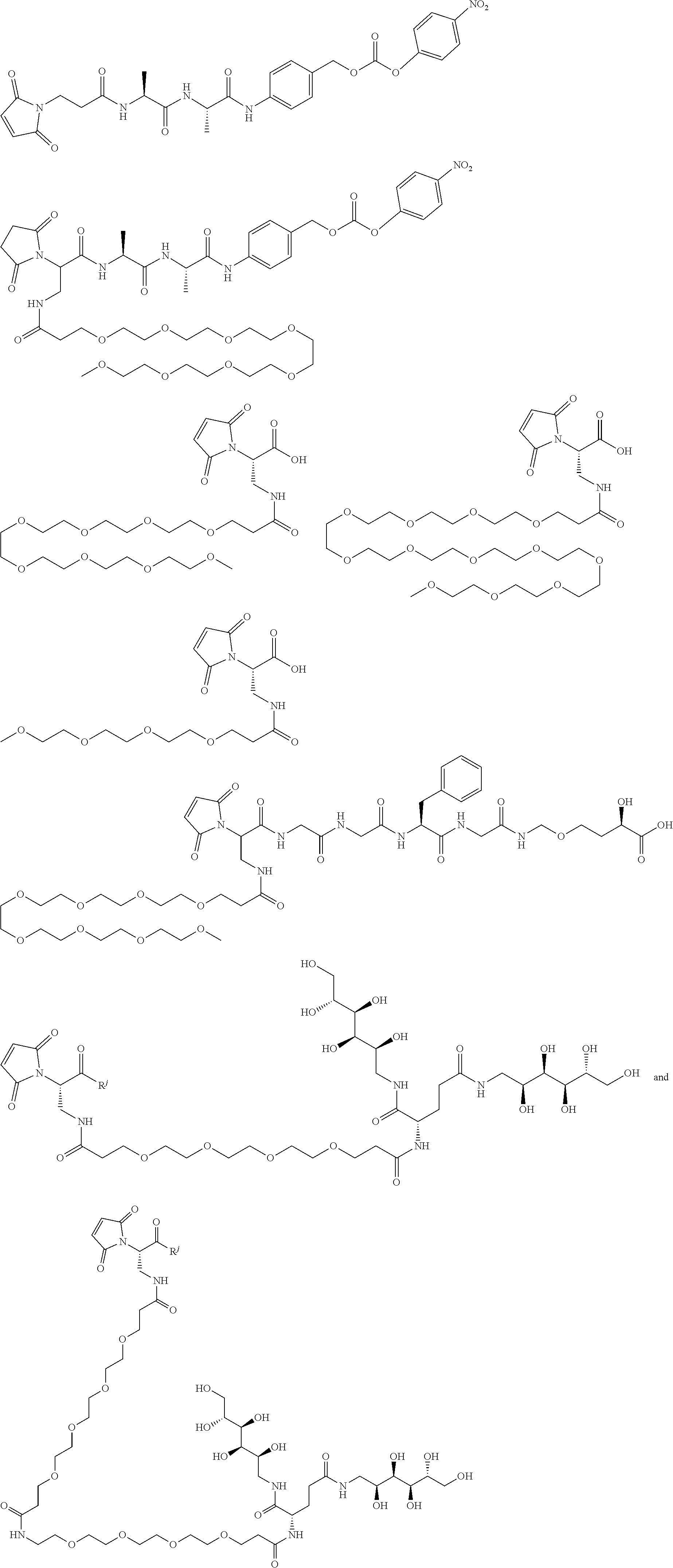
wherein X^{1a} is a PEG of 1 to 24 —CH₂CH₂O— subunits,
or a salt thereof.

28. A pharmaceutical composition comprising a compound of claim 1, or a pharmaceutically acceptable salt thereof, and a pharmaceutically acceptable carrier.

29. A method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer, colon cancer, rectal cancer, colorectal cancer, leukemia, bone cancer, skin cancer, thyroid cancer, pancreatic cancer, and lymphoma in a subject in need thereof, said method comprising administering to the subject in need of such treatment a therapeutically effective amount of a compound of claim 1, or a pharmaceutically acceptable salt thereof, or a pharmaceutical composition comprising said compound, salt or solvate thereof.

30. A method for treating and/or preventing a tumor, comprising administering to a patient in need thereof a therapeutically effective amount of the compound or pharmaceutical composition comprising the compound of claim 1, or a pharmaceutically acceptable salt thereof.
